SEQUENCE LISTING (40 sequences)

SEQ ID NO: 1 - DNA, 3573 bp, Homo sapiens
cctcgcagcggtctgcggctccttcccagcccccggcctagctctgcgaacggtgactgcccatccttggccgcaatgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggtcagggggcagggctgggccgctgccgtcgaggcgaggtcgaagcggccgtcgaggcggctgctcttgcctcccctcgcttcccctctccatcagcagcccaagggtgtggctccccttaccaacccaggtgtgtgcgggcagcatccctgcccacgggctccaagtgccccccgctacccctttgctctgagtgtttggggaggtgggagaagtgggtatctgtgcctcccctgagtaatgaggaaacccccttttcagctcccagtccgttagagacaatgcggggcaattccattagacagcctcagccctccatttagagtcctgggcagcagaacagcctctaaccggatcctggggggcgtgcggtctggggtgcgagctgggcggcgaccccgcagttcagcctctgcacgctcttcccgtcaggttggaggaggagacgcgcaagcgggaggacgcggagcacaacctcgtgctcttccgcaaggtgagtccgagcccctctccgagttcagcctccccaccgctacccccgatctcagtatccagaggtggcatcggtgggcgcggggagaagggggtaacccagatgcctcctgaggcagacagggaaggcctggtccttccttggtctgcgcagcccctaacttatcttgaacctccactgccacccctcgaaggacgtggacgatgccactctgtcccgcctggaactagagcgcaagattgagtctctgatggatgagattgagttcctcaagaagctgcacgaggaggtaagtgggcccggtatcaggggcggtttctgaggttgtggggtggtctcgctggagctggcgggtggagcggaggcatcgccctggggatcaggacgatgctgggtagacgcagcccctccaccctagtctacaggtggttagactcccacccttgcgccacctggcggcgggcagcggggctgtacctccgaaacctggcctctggtctcgcgcccgcgggggcgcagggctgtacgccctgccctccctggcgcccacttctgttcgttcaagcgtttcttctcttttctgtgcacgaactgcgtggcccgcgtggaatttgcgccgctgtccatcctctgcctgctcccggccgtaggagctgcgagacctgcaggtgagtgtggagagccagcaggtgcagcaggtggaggtggaagccacggtgaagcccgagctgacggcagcgctgagggacatccgcgcgcagtacgagagcatcgccgcgaagaacctgcaggaggcggaggagtggtacaagtccaaggtgcaagagccgggagggcctgcgaggcgggacgctggggtggtgtcgcgcgtcccagccgactaaagcctgggttacccccacttctcagtacgcggacctgtccgacgctgccaaccggaaccacgaggccctgcgccaggccaagcaggagatgaacgagtcccgacgccagatccagagtctaacgtgcgaggtggacgggctgcgcggcacggtgagtacgaagctgcgcgctcgggcccggggagcggacgatgaaatgttctgcaactggccccttccactctcctaccccagaacgaggcgctgctcaggcagttgagagagctggaggagcagttcgccctggaggcggggggctaccaggcgggcgctgcgcggctcgaggaggagctgcgacagctaaaagaggagatggcgcggcacctgagggagtaccaggagctcctcaacgtcaagatggccctggacatcgagatcgccacctaccgcaagctgctggagggcgaggagagccggtgagggtggagctgctggggcggggcagggcggggtcgggactgggccgggcagggcggggcctgggcaggggcgctgacaacttgcttcgcctctaggatctccgtgcccgtccattcttttgcctccttaaatataaagacgactggtgagtctggcttacagcctgtacctctccttgtccacttctgccctcctcgggctcttgctctggctcacctcagggatctcttctccccacggaacttctgggtcctggcctgtacccacccctactcctcaaacccgcccctcccctgccctcagggatagcagtgggagcctaagaggagagattcccacgccttcccccttgaaccctttatcctgctttcttcagtgcctgaggtggagcctccccaggacagccacagccggaagacggttctgatcaagaccattgagacccggaatggggaggtgaggcaggtcccctaatgccaggaccccaccatttcccatattatttctgagccccagcctggctgtcgctaatcttacttagggtgggtaattcttggggagagacagagaaggagacagagaacgtggatagataaccaggagcctctgctggttaccccagggagtggggctctatgatccaaaggagtaggatggagggtggcgctgaatggcttgtgcccatcatatgccctgtccccagcaggtggtgacagagtcccagaaggagcagcgcagtgagctggacaagtcttctgcccacagttactgaaccccttggtccggagccttgactctgccctaggcctgctcaaagcccaaaccctaagaccactcctgaattgtctcctctccctctgcatgtgtctaaaaggtggtaccaggcatccctttcctggcttatggccaagccctacccggccagcagtcgctgggcctctccctgccctgacacttgatgtgacctatgtgcttcccttttcatgtcccgataagaagccaatgatcccccctcaggacaaatctactccagccacgatgagaagtgggtgagccagggtctgagtttcacatttgaaccaaataaaatgctgtcaagagaaaactctccagtgca

SEQ ID NO: 2 - DNA, 2285 bp, Homo sapiens
cctcgcagcggtctgcggctccttcccagcccccggcctagctctgcgaacggtgactgcccatccttggccgcaatgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggttggaggaggagacgcgcaagcgggaggacgcggagcacaacctcgtgctcttccgcaaggacgtggacgatgccactctgtcccgcctggaactagagcgcaagattgagtctctgatggatgagattgagttcctcaagaagctgcacgaggaggtaagtgggcccggtatcaggggcggtttctgaggttgtggggtggtctcgctggagctggcgggtggagcggaggcatcgccctggggatcaggacgatgctgggtagacgcagcccctccaccctagtctacaggtggttagactcccacccttgcgccacctggcggcgggcagcggggctgtacctccgaaacctggcctctggtctcgcgcccgcgggggcgcagggctgtacgccctgccctccctggcgcccacttctgttcgttcaagcgtttcttctcttttctgtgcacgaactgcgtggcccgcgtggaatttgcgccgctgtccatcctctgcctgctcccggccgtaggagctgcgagacctgcaggtgagtgtggagagccagcaggtgcagcaggtggaggtggaagccacggtgaagcccgagctgacggcagcgctgagggacatccgcgcgcagtacgagagcatcgccgcgaagaacctgcaggaggcggaggagtggtacaagtccaaggtgcaagagccgggagggcctgcgaggcgggacgctggggtggtgtcgcgcgtcccagccgactaaagcctgggttacccccacttctcagtacgcggacctgtccgacgctgccaaccggaaccacgaggccctgcgccaggccaagcaggagatgaacgagtcccgacgccagatccagagtctaacgtgcgaggtggacgggctgcgcggcacgaacgaggcgctgctcaggcagttgagagagctggaggagcagttcgccctggaggcggggggctaccaggcgggcgctgcgcggctcgaggaggagctgcgacagctaaaagaggagatggcgcggcacctgagggagtaccaggagctcctcaacgtcaagatggccctggacatcgagatcgccacctaccgcaagctgctggagggcgaggagagccggatctccgtgcccgtccattcttttgcctccttaaatataaagacgactgtgcctgaggtggagcctccccaggacagccacagccggaagacggttctgatcaagaccattgagacccggaatggggaggtggtgacagagtcccagaaggagcagcgcagtgagctggacaagtcttctgcccacagttactgaaccccttggtccggagccttgactctgccctaggcctgctcaaagcccaaaccctaagaccactcctgaattgtctcctctccctctgcatgtgtctaaaaggtggtaccaggcatccctttcctggcttatggccaagccctacccggccagcagtcgctgggcctctccctgccctgacacttgatgtgacctatgtgcttcccttttcatgtcccgataagaagccaatgatcccccctcaggacaaatctactccagccacgatgagaagtgggtgagccagggtctgagtttcacatttgaaccaaataaaatgctgtcaagagaaaactctccagtgca

SEQ ID NO: 3 - DNA, 30 bp, Homo sapiens
gtaagtgggcccggtatcaggggcggtttc

SEQ ID NO: 4 - protein, 10 aa, Homo sapiens
VSGPGIRGGF

SEQ ID NO: 5 - DNA, 735 bp, Homo sapiens
  exon (1)..(732)
atgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggttggaggaggagacgcgcaagcgggaggacgcggagcacaacctcgtgctcttccgcaaggacgtggacgatgccactctgtcccgcctggaactagagcgcaagattgagtctctgatggatgagattgagttcctcaagaagctgcacgaggaggtaagtgggcccggtatcaggggcggtttctga

SEQ ID NO: 6 - DNA, 5658 bp, Homo sapiens
gcacacagccatccatcctcccccttccctctctcccctgtcctctctctccgggctcccaccgccgccgcgggccggggagccaccggccgccaccatgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggtgcggcacggccagaaacacaggggggcgggggactcgagcaagggggggagttggtgcgcccagaaagtggagaccaggggtggtgcggctgcacgcagctcttagggatagggcttggctccttggccactgtgtgggaggggtggggcacttgaagggcgtgagtgcgggcgccactgtagtctgggagtgtgctccgtgctgctgcaccggcgttccgcattaaagctgcccagcccttgttgggtgggggaggggaagacgtgggaattgggcgttgcctccggcctgcagtgagatcagctctctactgacctgcattaaccacaagttactttgcagcaactatcggatcatctagttaataaatagtagagtgaacaactctcaattaattctgaaggattactgtgaccagcatgctttatgactagttttaccaaccactcccttcctttatttagtaggtagacaggaaaatagtcaacattgttttaggtagttaactagtgatgttcatagtaaaccatttccttttaccttttttttttctttttttctttatgtgtaaaatcttctacaacatttctgtttaaacatctccatcttctggggagtagaaaaaatacaattttaaaaagatctccattttaaaacatctccatcttctggggagtagaaatttttttcttcttctggggagtagaaaaaataatttagatacataggaaatatttcatagaaaataatttttttcttttttttgtttacatctggctattttcttctcataaagaaaggcattagtttcctggcatgtaacccagctaaagaagagttaatcagtgaatgagagacacagtttttctatcaacttagtctgtttgcatgcattttatgatgatcattaaacagtattaagtaaagaaacagaagaacagaattttcgtccatcttttttttcatctcaggcttcatgaacttgggtattttaggcatgaaggtttttcaaaagatacaggaagttattctaggagagattttatcaaaggtgtgcaccttgattttaatcgaaactaggcctttgcaactacactacagtaaaataatagaagggatttatgctcggattttttttttgttttatttttgtcttcaaacaggacacgatcaacaaattagaaaatgaattgaggaccacaaagagtgaaatggcacgatacctaaaagaataccaagacctcctcaacgtgaagatggctttggatattgagattgcagcttacaggtgaaaatagaggggcaaagacagcagccattaaaccttaggaagaaaatcagatcccatttaaagttatgttggatcagaaaccttcaataatagtccttttgaaataatgaagtgttagtttttggcttcttccaagaagagggtatttagatatataagaatttaaccctgtaattaggagtcctgtttttatcttgtcattacactttaaatctaataggatgatttatttatattttttctggtctccatcaaaagatccccaggcattaagtattgataaatcccagccctgctcctgcttgcctttgtgtttagggtactcagagcaagttgtgaaacacaggtgttttttaacctcaccttgcatctgcatccccaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagccccccctctgaaggagaagccgaggaggaggagaaggacaaggaagaggccgaggaagaggaggcagctgaagaggaagaaggtatgataagaaaaaacccctgcaacttcaagtgtaaactgggtgtggagatttgttaggaggtggataagacaaatgaagccttgctcatttattcatatatgacattagaatcataaataaattttctgtttgtttagcaaaactttcctaaggcatctactctgaatgaggtgattggtcaaaattttcattttttaatataatcatttaacacagcaggttggtgtcctaaagaacaaaaatagataccagacacataatgaaagaaatattgaggttaagtcttggagaggagcagagcttcccatacctagaagtgatctcattcgatttaaatatgtgttcagtggcaaattattcatggcaagctttgtctgttacatgtgcttttggagagagtggagctgggaggttttggtagcattctgacagttgtgtttgcaaataaaacctttgcagacatgttttgactggacttaccctggatttgcattttgtacattttctttttatgttaaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattgaacccccatttccttaattatttcaggaataattctcccgaaatcaggtcaaccccatcaccaaccaaccaaccagttgagttccagattctatgtgaattaaaaagtcaatatatgtataattctgagatgacttaggttggacattcaatgttgtgctatgaatttcctctttatgcagagtatctgtttgcttgcagagtggctttctggcttgctgccagcctgtgcatggaccacgcttatgagttcaggatctacggcaatgtgaatcattcagatgtttacaataaaaaacaccacatgagtaaatgaattcactaatgttaatgttaaacttcatggaaaaatagtcctttgaaccttcggtggttagcaattaaagaccctgagttatgtgcaataaatagtaaataaagttataccgaatgatgtattttttgctgtggttgttacttaattaaaataccttaaagatggcaccaatataaagtatataccagtggactattgacctccaattttttaaaaagttgaaattttaacaattaccaatacttttttttcttccttcaattggaaattctgagggatacagttatgtcatgattacttgtgaaatctctccccacaaaaattttactgataataaacatgagtaaatgagaaagtctataaataacaatagactttgcctcataacacgtgttgtgttaggttttgggatggctttctagggagcaattctgaaattacttgacagatacactctttatggtaaacaggtcatttcagagtctagagtgctaaccattacactatggaaccatgataaataggtcatttcaaactgatatgtcacaatgcaatatggatagaatatgaatcccatacattcatccctataactgcattttaaaatatagtctaaatatcatgggaggaggttaaaacctgagaacatggtacaataagaaggaataattttttatctatatataatttcaggagaagggattaaaactaccaaaggttatttgccactccactgcaaacatgttgcctgcaatcctgagctcatgatttagtgtgttgttaagcgtaattctgttgcctcaagtaaataccacttaatgctacacatttttaaacctttaaaagctacagacacaagtaatgaaattatgagtcacttaaggggggaggtaatatgcttgtatgttacattgacattgctctgcatgtctctactgggagatatttggatttcaatgatagcttgactggttggacattgcatcaaatacacgccccatgtatcctttctgtcgtaaagttaagaacgctcttgcatttgatgtggaataaatatagatgatattactatctgctattctgcctttgttctgaaacaattgctttgtcagctaatttcattcaatatttgttttttagacgatctctataagttattaatttaacctgaaaccaaagtggtctccatttaaagttacttaatccctttgtaccacctatttctagttaaatatatgttgctatgcaaataggtaaagtgcttccttgccatgatggtaatggattggaactatgaaggctctcagtgtattggcttctgtaaagatgaggcgtctcctcagaaacaaaacttttcacatttctgcttactagacctgggttgatgtacatggtaagtctcaaacagatgcaagctatgtgcaaaaagtaactttagccaaatggaaatagctggatgctttgagaattacttggttgaagtaagaaaactgtaccatcctctatcctgtgtgccataataaaatagtaaaaaacctaa

SEQ ID NO: 7 - DNA, 1461 bp, Homo sapiens
  exon (1)..(1458)
atgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattga

SEQ ID NO: 8 - protein, 10 aa, Homo sapiens
NDLKSIRDLR

SEQ ID NO: 9 - DNA, 1353 bp, Homo sapiens
  exon (1)..(1350)
atgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgttatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattga

SEQ ID NO: 10 - protein, 10 aa, Homo sapiens
ARKGAKNTDA

SEQ ID NO: 11 - DNA, 20 bp, Homo sapiens
gtggacgatgccactctgtc

SEQ ID NO: 12 - DNA, 20 bp, Homo sapiens
ggtccgcgtactgagaagtg

SEQ ID NO: 13 - DNA, 20 bp, Homo sapiens
tcgacttctccatggccgag

SEQ ID NO: 14 - DNA, 21 bp, Homo sapiens
cgagtccagaggccaggtttc

SEQ ID NO: 15 - DNA, 20 bp, Homo sapiens
gtggacgatgccactctgtc

SEQ ID NO: 16 - DNA, 19 bp, Homo sapiens
ctggtactccctcaggtgc

SEQ ID NO: 17 - DNA, 22 bp, Homo sapiens
ctccaattggcctgctgacgtc

SEQ ID NO: 18 - DNA, 22 bp, Homo sapiens
ggacttgtcgtaccagcgattg

SEQ ID NO: 19 - DNA, 56 bp, Artificial Sequence
  Synthetic - Per3,4 siRNA-1-F
tcgaggtctacaggtggttagactggaattcgagtctaaccacctgtagacttttt

SEQ ID NO: 20 - DNA, 56 bp, Artificial Sequence
  Synthetic - Per3,4 siRNA-1-R
ctagaaaaagtctacaggtggttagactcgaattccagtctaaccacctgtagacc

SEQ ID NO: 21 - DNA, 56 bp, Artificial Sequence
  Synthetic - Per 3,4 siRNA-2-F
tcgagttctgttcgttcaagcgttggaattcgaacgcttgaacgaacagaattttt

SEQ ID NO: 22 - DNA, 56 bp, Artificial Sequence
  Synthetic - Per3,4 siRNA-2-R
ctagaaaaattctgttcgttcaagcgttcgaattccaacgcttgaacgaacagaac

SEQ ID NO: 23 - DNA, 56 bp, Artificial Sequence
  Synthetic - NFL-60 siRNA-F
tcgagctcaagtccatccgcgaccggaattcgggtcgcggatggacttgagttttt

SEQ ID NO: 24 - DNA, 56 bp, Artificial Sequence
  Synthetic - NFL-60 siRNA-F
ctagaaaaactcaagtccatccgcgacccgaattccggtcgcggatggacttgagc

SEQ ID NO: 25 - DNA, 56 bp, Artificial Sequence
  Synthetic - NFL-57 si-RNA-F
tcgagcaaaggcgccaagaacaccggaattcgggtgttcttggcgcctttgttttt

SEQ ID NO: 26 - DNA, 56 bp, Artificial Sequence
  NFL-57 siRNA-R
ctagaaaaacaaaggcgccaagaacacccgaattccggtgttcttggcgcctttgc

SEQ ID NO: 27 - DNA, 1924 bp, Homo sapiens
cctcgcagcggtctgcggctccttcccagcccccggcctagctctgcgaacggtgactgcccatccttggccgcaatgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggttggaggaggagacgcgcaagcgggaggacgcggagcacaacctcgtgctcttccgcaaggacgtggacgatgccactctgtcccgcctggaactagagcgcaagattgagtctctgatggatgagattgagttcctcaagaagctgcacgaggaggagctgcgagacctgcaggtgagtgtggagagccagcaggtgcagcaggtggaggtggaagccacggtgaagcccgagctgacggcagcgctgagggacatccgcgcgcagtacgagagcatcgccgcgaagaacctgcaggaggcggaggagtggtacaagtccaaggtgcaagagccgggagggcctgcgaggcgggacgctggggtggtgtcgcgcgtcccagccgactaaagcctgggttacccccacttctcagtacgcggacctgtccgacgctgccaaccggaaccacgaggccctgcgccaggccaagcaggagatgaacgagtcccgacgccagatccagagtctaacgtgcgaggtggacgggctgcgcggcacgaacgaggcgctgctcaggcagttgagagagctggaggagcagttcgccctggaggcggggggctaccaggcgggcgctgcgcggctcgaggaggagctgcgacagctaaaagaggagatggcgcggcacctgagggagtaccaggagctcctcaacgtcaagatggccctggacatcgagatcgccacctaccgcaagctgctggagggcgaggagagccggatctccgtgcccgtccattcttttgcctccttaaatataaagacgactgtgcctgaggtggagcctccccaggacagccacagccggaagacggttctgatcaagaccattgagacccggaatggggaggtggtgacagagtcccagaaggagcagcgcagtgagctggacaagtcttctgcccacagttactgaaccccttggtccggagccttgactctgccctaggcctgctcaaagcccaaaccctaagaccactcctgaattgtctcctctccctctgcatgtgtctaaaaggtggtaccaggcatccctttcctggcttatggccaagccctacccggccagcagtcgctgggcctctccctgccctgacacttgatgtgacctatgtgcttcccttttcatgtcccgataagaagccaatgatcccccctcaggacaaatctactccagccacgatgagaagtgggtgagccagggtctgagtttcacatttgaaccaaataaaatgctgtcaagagaaaactctccagtgca

SEQ ID NO: 28 - DNA, 936 bp, Homo sapiens
atgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggttggaggaggagacgcgcaagcgggaggacgcggagcacaacctcgtgctcttccgcaaggacgtggacgatgccactctgtcccgcctggaactagagcgcaagattgagtctctgatggatgagattgagttcctcaagaagctgcacgaggaggagctgcgagacctgcaggtgagtgtggagagccagcaggtgcagcaggtggaggtggaagccacggtgaagcccgagctgacggcagcgctgagggacatccgcgcgcagtacgagagcatcgccgcgaagaacctgcaggaggcggaggagtggtacaagtccaaggtgcaagagccgggagggcctgcgaggcgggacgctggggtggtgtcgcgcgtcccagccgactaa

SEQ ID NO: 29 - protein, 21 aa, Homo sapiens
VQEPGGPARRDAGVVSRVPAD

SEQ ID NO: 30 - DNA, 25 bp, Homo sapiens
gacaggtccgcgtactgagaagtgg

SEQ ID NO: 31 - DNA, 24 bp, Homo sapiens
gtccaaggtgcaagagccgggagg

SEQ ID NO: 32 - DNA, 19 bp, Homo sapiens
ctggatcgttgatgcccag

SEQ ID NO: 33 - DNA, 20 bp, Homo sapiens
cggctctcggacacctcgtc

SEQ ID NO: 34 - DNA, 16 bp, Homo sapiens
gtaaaacgacggccag

SEQ ID NO: 35 - DNA, 17 bp, Homo sapiens
caggaaacagctatgac

SEQ ID NO: 36 - DNA, 37 bp, Homo sapiens
ggccacgcgtcgactagtacttttttttttttttttt

SEQ ID NO: 37 - DNA, 20 bp, Homo sapiens
ggccacgcgtcgactagtac

SEQ ID NO: 38 - DNA, 36 bp, Homo sapiens
ataagaatgcggccgcgcacacagccatccatcctc

SEQ ID NO: 39 - DNA, 33 bp, Homo sapiens
cgcggatccgcgttcggtataactttatttact

SEQ ID NO: 40 - DNA, 20 bp, Homo sapiens
gcacacagccatccatcctc

(19) United States
(12) Patent Application Publication
(10) Pub. No.: US 2009/0053723 A1
(43) Pub. Date: Feb. 26, 2009

(54) Peripherin and Neurofilament Light Protein Splice Variants in Amyotrophic Lateral Sclerosis (ALS)

(76) Inventors: Shangxi Xiao, Toronto (CA); Janice Robertson, Toronto (CA)

Correspondence Address: BERESKIN AND PARR, 40 KING STREET WEST, BOX 401, TORONTO, ON, M5H 3Y2, CA

(21) Appl. No.: 12/196,622

(22) Filed: Aug. 22, 2008

Related U.S. Application Data
(60) Provisional application No. 60/957,830, filed on Aug. 24, 2007.

(51) Int. Cl. C12Q 1/68; C12N 15/00; C12N 15/11; C07H 21/04; G01N 33/53; C07K 7/00; C07K 16/18

(52) U.S. Cl. 435/6; 536/23.5; 435/320.1; 536/24.5; 530/328; 530/326; 530/387.9; 435/7.1; 435/7.21

(57) ABSTRACT

Nucleotide sequences encoding novel splice variants of peripherin and neurofilament light protein, proteins encoded by the novel splice variants and antibodies thereto are disclosed. In addition, methods are described for detecting ALS in a subject suspected of having ALS, comprising detecting the presence or absence of the novel splice variants or resulting proteins or a change in the amount of the novel splice variants or resulting proteins; wherein the presence or change in the amount of the nucleotide sequence is indicative of ALS.

This application claims the benefit under 35 USC §119(e) of U.S. provisional application No. 60/957,830 filed Aug. 24, 2007.

### FIELD OF THE DISCLOSURE

The disclosure relates to novel splice variants of peripherin and neurofilament light protein. In particular, the disclosure relates to methods of detecting and diagnosing ALS through detection of the novel splice variants.

### BACKGROUND OF THE DISCLOSURE

Amyotrophic lateral sclerosis (ALS) is an adult-onset neurodegenerative disease affecting motor neurons of the motor cortex, brain stem and spinal cord. Approximately 5-10% of cases are inherited (familial; fALS) with the remainder occurring sporadically (sporadic; sALS). Mutations in the gene encoding superoxide dismutase-1 (SOD1) are causative of approximately 20% of fALS cases, corresponding to 1-2% of all ALS cases (Rosen et al., 1993). Sporadic and familial ALS are clinically and pathologically indistinguishable, sharing a number of intraneuronal inclusion bodies that are characteristic of the disease. These include ubiquitinated inclusions that comprise skein-like inclusions, round inclusions and Lewy body-like inclusions (Hays, 2006; Ince et al., 1998; Xiao et al., 2006). Hyaline conglomerate inclusions are also ubiquitinated, but appear to be more closely linked with fALS cases carrying mutations in SOD1 (Ince et al., 1998; Hays et al., 2006).

Neurofilaments are neuronal intermediate filaments (nIFs) comprised of three subunits, neurofilament-light (NF-L), neurofilament-medium (NF-M) and neurofilament-heavy (NF-H). NF-L forms the core of the neurofilament structure co-assembling with NF-H and/or NF-M to form a filamentous network in neuronal cells.

A prominent pathological hallmark of both familial and sporadic ALS is the presence of intraneuronal inclusions composed of disorganized masses of neurofilaments (Carpenter, 1968). These include both hyaline conglomerate inclusions and axonal spheroids. How these accumulations/aggregations are formed is unknown, and their involvement in disease pathogenesis remains an enigma. Deletions and insertions within the mutliphosphorylation region of the C-terminal domain of NF-H have been found in ˜1% of sporadic ALS cases, but how these are related to disease is not clear (Figlewicz et al., 1993; Tompkins et al., 1998; Al-Chalabi et al., 1999). NFL mutations in humans have been linked to Charcot-Marie-Tooth disease (Lee et al., 1994; Mersiyanova et al., 2000; De Jonghe et al., 2001), and transgenic mice with single missense mutations in NFL are shown to develop motor neuron degeneration and neurogenic atrophy associated with neurofilament accumulation similar to ALS (Schwartz et al., 1995). Nevertheless, the cause of aggregation and the specific role(s) of neurofilaments in ALS pathogenesis remain unclear and no well-established NF mutation has been identified in ALS patients.

Peripherin, a neuronal intermediate filament protein, is associated with ubiquitinated inclusions, specifically round inclusions and Lewy body-like inclusions, hyaline conglomerate inclusions as well as with axonal spheroids, large swellings that occur in proximal axons of diseased motor neurons (Xiao et al., 2006; Corbo et al., 1992; He et al., 2004; Migheli et al., 1993).

The present inventors have previously shown the deregulated expression of peripherin splice variants in transgenic mouse models of ALS (Robertson et al., 2003). In particular, the abnormal expression of a neurotoxic splice variant of peripherin, Per 61, was shown in motor neurons of mutant SOD1 transgenic mice (Robertson et al., 2003). This splice variant was generated by the retention of intron 4 and it's upregulated expression induced peripherin aggregate formation (Robertson et al., 2003; Landon et al., 1989; Landon et al., 2000).

### SUMMARY OF THE DISCLOSURE

The present inventors have shown that there is deregulated peripherin splicing in ALS, with an upregulation of a transcript with intron 3 and 4 retained designated Per 3,4, resulting in a 28 kD splice variant designated Per 28. The present inventors have shown that this Per 28 splice variant is associated with intraneuronal inclusion bodies in ALS. Thus, deregulated peripherin splice variant expression may cause peripherin to aggregate and form inclusion bodies in ALS.

The present inventors have also shown by RT-PCR the expression of two alternatively spliced, in-frame variants of the neurofilament light (NFL) gene in human ALS spinal cord tissue: NFL-60 and NFL-57; the former appears to be disease specific while the latter is found in both normal and pathogenic settings. Both isoforms are products of alternative processing at splice sites found on exon 1. The inventors further provide evidence for the role of NFL-57 and NFL-60 in promoting intraneuronal inclusion by demonstrating the lack of NF filamentous network and the presence of NF aggregation in SW13 vim(−) cells co-transfected with NFL-60 or NFL-57 with NFH or NFM. This implies that perturbation in NFL metabolism and its respective ratio to other neurofilament subunits, as well as disruption of alternative splicing regulation may contribute to the pathogenesis of ALS.

One aspect of the disclosure is the nucleic acid sequence comprising the retained intron 3 and intron 4 of the peripherin gene as shown in SEQ ID NO:2. The location of introns 3 and 4 is underlined in the genomic sequence shown in SEQ ID NO 1. Another aspect is a nucleic acid sequence consisting essentially of SEQ ID NO:2. Another aspect of the disclosure is an siRNA that targets the nucleic acid sequence as shown in SEQ ID NO:2. In one embodiment, the location of the siRNA1 sequence is from nucleotides 831 to 849 and the location of the siRNA2 sequence is from nucleotides 964 to 982 of SEQ ID NO:2. In one embodiment, forward and reverse primers as shown in SEQ ID NOs: 19-22 are used to create the siRNAs. A further aspect of the disclosure is a nucleic acid sequence encoding the Per 28 splice variant protein as shown in SEQ ID NO:5, an amino acid sequence comprising SEQ ID NO:5 and antibodies that are specific for the Per 28 protein.

Another aspect of the disclosure is the nucleic acid sequence comprising the retained intron 4 of the peripherin gene as shown in SEQ ID NO:27. Another aspect is a nucleic acid sequence consisting essentially of SEQ ID NO:27. Another aspect of the disclosure is an siRNA that targets the nucleic acid sequence as shown in SEQ ID NO:27. A further aspect of the disclosure is a nucleic acid sequence encoding the Per 32 splice variant as shown in SEQ ID NO:28, an amino acid sequence comprising SEQ ID NO:28 and antibodies that are specific for the Per 32 splice variant.

Another aspect of the disclosure is the nucleic acid sequence comprising NFL-60 as shown in SEQ ID NO:7. NFL-60 results from the deletion of a region of exon 1 as shown in SEQ ID NO:6. A further aspect of the disclosure is the nucleic acid sequence comprising NFL-57 as shown in SEQ ID NO:9. Another aspect of the disclosure is an siRNA that targets the nucleic acid sequence as shown in SEQ ID NOs:7 or 9. In one embodiment, the location of the siRNA sequence for NFL-60 is from nucleotides 247 to 265 of SEQ ID NO:7. In one embodiment, forward and reverse primers as shown in SEQ ID NOs: 23 and 24 are used to create the siRNA. In another embodiment, the location of the siRNA sequence for NFL-57 is from nucleotides 588 to 606 of SEQ ID NO:9. In one embodiment, forward and reverse primers as shown in SEQ ID NOs: 25 and 26 are used to create the siRNA. A further aspect of the disclosure is an amino acid sequence encoding NFL-60 as shown in SEQ ID NO:7 and antibodies that are specific to NFL-60. Another aspect of the disclosure is an amino acid sequence encoding NFL-57 as shown in SEQ ID NO:9 and antibodies that are specific to NFL-57.

A further aspect of the disclosure is a method to diagnose, detect and monitor whether a subject is at risk of developing ALS or whether a subject has ALS comprising detecting the presence, decrease or increase of Per 28, NFL-60 or NFL-57 in the subject, wherein detection or an increase of Per 28, NFL-60 or NFL-57 is indicative of the subject being at risk of developing ALS or is indicative of the person having ALS.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the Identification of a peripherin mRNA transcript retaining introns 3 and 4. (A) Schematic showing locations of forward primer F1 in exon 3 and reverse primer R1 in intron 4 used for RT-PCR amplification of transcripts retaining intron 4. (B) Off target primer locations F1 in exon 1 and R2 in intron 3 used to control for genomic contamination. (C) A major RT-PCR product of 701 bp was amplified from human dorsal root ganglion RNA using the primer pair indicated in (A) corresponding to a transcript retaining introns 3 and 4; a minor transcript of 339 bp encompassing intron 4 was also identified as well as product of 550 bp which could not be identified. (D) The primer pair in (B) amplified a product of 683 bp corresponding to a transcript in which introns 1 and 2 were spliced out, confirming that the samples were not contaminated with genomic DNA. All RT-PCR products were confirmed by sequencing.

FIG. 2 shows the full-length peripherin transcript retaining introns 3 and 4 obtained by 5′ and 3′RACE. (A) Locations of primers for 5′ and 3′RACE within the full-length peripherin gene. GSP refers to the gene specific primers. (B) 5′RACE gave one major product corresponding to the peripherin sequence from the 5′end and retained introns 3 and 4 (lane 1); 3′RACE gave one product corresponding to the retention of part of intron 4 and the remaining normal peripherin sequence to the 3′end (lane 2).

FIG. 3 shows the expression of Per 3,4 cDNA in transfected cells (A) Immunoblot of increasing loadings (10 μg, 20 μg, 50 μg, 100 μg) of cell lysates derived from SW13vim(−) cells transfected with the peripherin gene (PG; Lanes 1,3,5,7) or the Per 3,4 cDNA (P34; Lanes 2,4,6,8), probed with peripherin polyclonal antiserum, AB1530. Samples harvested after 48 h expression. The 28 kDa species predicted from the Per 3,4 cDNA sequence is indicated by the large arrow in Lane 2. This species was undetectable in similar loadings of PG transfected cells (Lane 1). Note also the additional peripherin species of ˜48 kDa, 45 kDa, 32.5 kDa and 25 kDa, indicated by small arrows (Lane 2). All of these species were apparent in the Per 3,4 transfected cell lysates at 10 μg loadings. The only species apparent in 10 μg loadings of the peripherin gene transfected cell lysates was of 58 kDa, corresponding to the constitutively spliced peripherin species (Lane 1). This was also expressed form the Per 3,4 cDNA, indicating that Per 3,4 is itself alternatively spliced. However, as protein loadings were increased, the additional peripherin species observed in Lane 2 also became apparent in the peripherin gene transfected cell lysates (indicated by arrows in Lane 7). (B) Validation of Per 28 kDa species as a product derived from transcript retaining intron 3. Total protein lysates from SW13vim(−) cells expressing the human peripherin gene (PG) or the Per 3,4 cDNA (P34) probed by immunoblotting with the Per 3,4 splice variant specific antibody revealed a single band of 28 kDa (indicated by arrow). This species was more robustly expressed from the Per 3,4 cDNA than from the peripherin gene.

FIG. 4 shows immunofluorescence labeling of SW13vim(−) cells transfected with (A) the normal peripherin gene compared with (B) the Per 3,4 cDNA, labeled with polyclonal peripherin antibody (AB1530). Note the normal filamentous distribution of peripherin in (A) and the large amorphous aggregates (indicated by arrows) in (B). Bar, 20 μm.

FIG. 5 shows downregulation of Per 3,4 using RNAi prevents peripherin aggregate formation. (A) RT-PCR of RNA isolated from SW13vim (−) cells transfected with Per 3,4 (Lane 1); Per 3,4 and RNAi-1 (Lane 2); and Per 3,4 and RNAi-2 (Lane 3). Samples were normalized to beta-actin. Note the downregulation of Per 3,4 message in cells co-transfected with RNAi-2 (Lane 3). (B) Immunoblot of total cell lysates of cells transfected with Per 3,4 (Lanes 1) or co-transfected with Per 3,4 and RNAi-2 (Lanes 2), probed with AB1530. Note the reduction in expression of the 28 kDa Per 3,4 species at 24 h and 48 h after expression (Lanes 2, indicated by large arrow) and the maintained expression of the normal peripherin product (indicated by small arrow in Lanes 1 and 2). (C) and (D) Immunofluorescence labeling of SW13vim(−) cells expressing Per 3,4 (C) or Per 3,4 and RNAi-2 (D), labeled with AB1530. Note the peripherin aggregates in (C) and the predominance of filamentous networks in (D). Bar, 35 μm.

FIG. 6 shows upregulated expression of Per 3,4 mRNA in ALS versus control tissue. RT-PCR of total RNA isolated from spinal cords of control (Lanes 1 and 2) and ALS (Lanes 3 and 4) cases. Samples were normalized to beta-actin or to ChAT (to normalize for neuronal content). Note the reduction in ChAT RT-PCR product in ALS samples (Lanes 3 and 4) compared to controls Lanes 1 and 2) in the reactions normalized to beta-actin, consistent with loss of motor neurons in ALS. The normal peripherin message was amplified using the primers shown in Table 1 (PRPH). PRPH appeared decreased in ALS samples (Lanes 3 and 4) compared to controls (Lanes 1 and 2) when RT-PCR reactions were normalized to beta-actin, but increased when normalized to ChAT. An increase in Per 3,4 expression was detected in ALS samples compared to controls whether the samples were normalized to beta-actin or to ChAT.

FIG. 7 shows upregulated expression of Per 28 kDa in ALS versus control tissue. (A) Triton X-100 (30-40 μg) preparations from lumbar spinal cord of four independent ALS and control cases were probed by immunoblotting with the Per 28 kDa splice variant specific antiserum. Signal for the Per 28 kDa species was detected in the ALS samples but not in the controls. (B) The immunoblot in (A) was stripped and reprobed with AB1530, the commercially available antiserum, revealing the constitutively spliced Per 58 kDa peripherin species in all samples.

FIG. 8 shows immunocytochemical labeling of ALS lumbar spinal cord with Per 28 specific antiserum. (A-C) Three examples of motor neurons containing Per 28 immunoreactive inclusion bodies (indicated with arrows). Bar, 20 μm

FIG. 9 shows evidence of alternative splicing of NF-L in control and ALS spinal cord tissue (A) Location of primers for RT-PCR of NF-L total RNA; (B) RT-PCR of total RNA from control (lanes 1 and 2) and ALS (lanes 3 and 4) lumbar spinal cord. N, represents the normal NF-L species; F1 and F2 represent alternatively spliced mRNA species in which there are in-frame deletions within exon 1 of the NF-L pre-mRNA. The full-length cDNAs for F1 and F2 were obtained by PCR and shown by sequencing to correspond to two mRNA species found on Genbank with accession numbers BC066952 and AK057731, respectively. Arrowhead indicates a non-specific PCR product. These cDNAs were amplified by RT-PCR. F1 corresponds to NFL-60. F2 corresponds to NFL-57.

FIG. 10 shows the protein structure of NFL-60 and NFL-57. The in-frame splicing deletions in exon 1 correspond to a loss of coil 1b, all of coil 2a and part of coil 2b in NFL-57. For NFL-60, there is a loss of coil 1a and part of coil 1b.

FIG. 11 shows the expression of NFL-60 and NFL-57 in SW13 cells (A) Plasmids encoding NFL-68, NFL-60 and NFL-57 were transfected into SW13 vim(−) cells and harvested after expression for 48 hours. 10 μg of total cell lysate was loaded onto a 10% SDS-PAGE gel, blofted onto PVDF membrane and probed with polyclonal anti-NF-L primary and anti-rabbit secondary antibodies. From the immunoblot, NFL, NFL-60 and NFL-57 were shown to have molecular weights of approximately 68 kDa, 60 kDa and 57 kDa respectively. (B) Transfected SW13 vim(−) cells expressing NFL-68, NFL-60 and NFL-57 were labeled immunocytochemically with polyclonal anti-NFL primary antibody and anti-rabbot IgG conjugated to Alexa Fluor 594. NFL-68 formed fine filamentous structures in some cells whereas NFL-60 and NFL-57 formed aggregates distributed throughout the cytoplasm. In some cases larger aggregates were observed (arrows).

FIG. 12 shows the assembly properties of NFL and its splice variants with NFH and NFM in SW13 vim(−) cells. Plasmids encoding NFL-68, NFL-60 and NFL-57 were individually co-transfected with plasmids encoding NFH or NFM in SW13 vim(−) cells and double-fluorescently labeled with polyclonal anti NFL, monoclonal anti-NFH (SMI31, SMI32) or monoclonal anti-NFM (NN18) antibodies, and with secondary antibodies conjugated with Alexa Fluor 594 (red) and 488 (green) for visulalizing NFL and NFH or NFM, respectively. Well-established filamentous networks throughout the cytoplasm were observed in cells co-transfected with NFL-68 and NFH or NFM. In contrast, NFL-60 and NFL-57 were incapable of network assembly with either NFH or NFM, instead forming punctate aggregates that were distributed throughout the cytoplasm. In some cases larger aggregates were also apparent, most predominantly in cells co-expressing NFL-60 or NFL-57 with NFM.

FIG. 13 shows the fractionation of two ALS spinal cord tissues (ALS-2 and ALS-2) shows expression of NFL-60 in the TX-100 insoluble pellet, indicating that NFL-60 is integrated within higher polymeric structures.

FIG. 14 shows (A) RT-PCR of total RNA isolated from one control case and eight ALS cases. The transcript encoding NFL-60 could be detected in three of the eight cases (indicated by arrows in A3, A4 and A8). NFL-57 was expressed both in control and ALS cases. (B) Immnoblot of TX-100 insoluble preparations form ALS and control spinal cords probed with antibody to NFL. Note detection of NFL-60 protein in the ALS cases A3 and A2. (C) The A2 and A3 cases were shown to have neurofilament pathology that coincided with the expression of NFL-60.

FIG. 15 shows the generation and purification of rabbit polyclonal antibody specific to NFL-60. Rabbit polyclonal antisera raised to synthetic peptide corresponding to the unique sequence in NFL-60 (NDLKSIRDLR (SEQ ID NO:8)). Approximately 10 μg loadings of total lysates from cells expressing NFL-68 (lanes 1, 3, 5) or NFL-60 (lanes 2, 4, 6) were resolved on 10% SDS-PAGE and transferred to PVDF membrane. The membrane was divided into three pieces and probed with the antibodies as shown: commercially available monoclonal to NFL, NR4; crude NFL-60 specific antisera; affinity purified NFL-60 antibody. NR4 antibody labels both NFL-68 and NFL-60 (Lanes 1 and 2) whereas the crude NFL-60 antisera shows increased specificity for NFL-60 (compare lanes 1 and 2 with 3 and 4). The slight cross reactvitiy of the NFL-60 antisera with NFL-68 (arrow in lane 3) is removed by affinity purification (note lack of signal in Lane 5).

FIG. 16 shows (A) the sandwich ELISA with serially diluted target protein and (B) the sandwich ELISA with CSF samples.

FIG. 17 shows standard curve for quantitative analysis using KLH-linked Per 3,4 peptide. This is a standard ELISA validating use of KLH-linked-Per28 peptide for use in developing quantitative sandwich ELISA.

FIG. 18 shows the generation and purification of rabbit polyclonal antibody specific for NFL-60.

### DETAILED DESCRIPTION OF THE DISCLOSURE

### (A) Nucleic Acids

The present inventors have demonstrated that an alternatively spliced variant of the peripherin protein is upregulated in ALS and is associated with inclusion bodies pathognomic of ALS.

Accordingly, an isolated nucleic acid molecule retaining intron 3 and 4 of the peripherin gene (hereinafter referred to as Per 3,4) is described and an isolated nucleic acid molecule retaining intron 4 of the peripherin gene is described.

The present inventors have also demonstrated that alternatively spliced variants of the NFL protein are also associated with ALS.

Accordingly, isolated nucleic acid molecules encoding the splice variants, NFL-60 and NFL-57, are described.

The term “isolated” refers to a nucleic acid substantially free of cellular material or culture medium when produced by recombinant DNA techniques, or chemical precursors, or other chemicals when chemically synthesized.

The term “nucleic acid molecule” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the Per 3,4 nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “peripherin” as used herein refers to the peripherin neuronal intermediate filament gene or protein from any species or source, preferably human. “Per 3,4” refers to the peripherin gene that retains intron 3 and 4. “Per 28” refers to the splice variant produced by the Per 3,4 gene. “Per 32” refers to the peripherin gene that retains intron 4 and the protein produced by that gene.

The term “NFL” as used herein refers to the neurofilament light gene or protein from any species or source, preferably human. “NFL-57” and “NFL-60” refer to two alternatively spliced variants of NFL, which result from alternative processing at splice sites in exon 1 of the NFL gene.

One aspect of the present disclosure is thus an isolated nucleic acid molecule comprising a nucleotide sequence selected from the group consisting of:

(a) a nucleic acid sequence as shown in SEQ ID NO:2, 27, 7, or 9, wherein T can also be U;

(b) a nucleic acid sequence that is complementary to a nucleic acid sequence of (a);

(c) a nucleic acid sequence that has substantial sequence homology to a nucleic acid sequence of (a) or (b);

(d) a nucleic acid sequence that is an analog of a nucleic acid sequence of (a), (b) or (c);

(e) a nucleic acid sequence that hybridizes to a nucleic acid sequence of (a), (b), (c) or (d) under stringent hybridization conditions; and

(f) a nucleic acid sequence differing from any of the nucleic acid sequences of (a) to (e) in codon sequences due to the degeneracy of the genetic code.

In the sequences referred to above, T can also be U. As previously stated, the disclosure includes isolated DNA molecules having such sequences of nucleotides, and RNA molecules having such sequences. The disclosure thus includes isolated mRNA transcribed from DNA having such a sequence. The disclosure further encompasses nucleic acid molecules that differ from any of the nucleic acid molecules of the disclosure in codon sequences due to the degeneracy of the genetic code.

The disclosure also encompasses nucleic acid sequences or molecules that are analogs of the nucleic acid sequences and molecules described herein. The term “a nucleic acid sequence which is an analog” means a nucleic acid sequence which has been modified as compared to the sequences described herein, such as sequences of (a), (b), (c) or (d), above wherein the modification does not alter the utility of the sequences described herein. The modified sequence or analog may have improved properties over the sequence shown in (a), (b), (c), or (d). One example of a modification to prepare an analog is to replace one of the naturally occurring bases (i.e. adenine, guanine, cytosine or thymidine) of the sequence shown in SEQ ID NO: 2, 27, 7 and 9 with a modified base such as such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecule shown in SEQ ID NO: 2, 27, 7 or 9. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

In another aspect, the present disclosure includes a fragment of the nucleotide sequence encoding peripherin, said fragment comprising intron 3 (nucleic acids 703 to 1063 of SEQ ID NO:2) or intron 4 (nucleic acids 1232 to 1322 of SEQ ID NO:2). In yet another aspect, the present disclosure includes a fragment of the nucleotide sequence encoding NFL-60, said fragment comprising a nucleic acid that encodes SEQ ID NO:8. In a further aspect, the present disclosure includes a fragment of the nucleotide sequence encoding NFL-57, said fragment comprising a nucleic acid that encodes SEQ ID NO:9. Such fragments can find usefulness as probes or depending on the fragments may even have biological activity themselves. The complement of the probe can find utility in, for example, manufacture of the probe or inhibition of any activity of the fragment, as the case may be. In a particular use, the probe can be used to determine the presence of an RNA molecule in a sample which might, or might not, also include an RNA molecule encoding Per 28, Per 32, NFL-60 or NFL-57. Such a probe would generally be 20 nucleotides long or be at least 20 nucleotides long. The probe could also be 25, 30, 35, 40, 45, 50, 55, 60 or more nucleotides in length and the probe can include the full length of the complement to the sequence to which it is intended to bind.

The disclosure includes the method of determining the presence of a nucleic acid molecule encoding Per 28, Per 32, NFL-60, or NFL-57 in a sample containing RNA isolated from a human cell, using such a probe.

In the context of this specification, the term “conserved” describes similarity between sequences. The degree of conservation between two sequences can be determined by optimally aligning the sequences for comparison. Sequences may be aligned using the Omiga software program, Version 1.13. (Oxford Molecular Group, Inc., Campbell, CA). The Omiga software uses the Clustal W Alignment algorithms [Higgins et al., 1989; Higgins et al., 1991; Thompson et al. 1994] Default settings used are as follows: Open gap penalty 10.00; Extend gap penalty 0.05; Delay divergent sequence 40 and Scoring matrix—Gonnet Series. Percent identity or homology between two sequences is determined by comparing a position in the first sequence with a corresponding position in the second sequence. When the compared positions are occupied by the same nucleotide or amino acid, as the case may be, the two sequences are conserved at that position. The degree of conservation between two sequences is often expressed, as it is here, as a percentage representing the ratio of the number of matching positions in the two sequences to the total number of positions compared.

In one aspect, the present disclosure is a nucleic acid molecule which encodes a protein that is a conservatively substituted variant of the protein encoded by the nucleotide sequence of SEQ ID NO:2, 27, 7 or 9.

Further, it will be appreciated that the disclosure includes nucleic acid molecules comprising nucleic acid sequences having substantial sequence homology with the nucleic acid sequence as shown in SEQ ID NO. 2, 27, 7, 9 or fragments thereof. The term “sequences having substantial sequence homology” means those nucleic acid sequences that have slight or inconsequential sequence variations from these sequences, i.e., the sequences function in substantially the same manner to produce functionally equivalent proteins. The variations may be attributable to local mutations or structural modifications. Peripherin and NFL are known to contribute to cell structural integrity. It has also been shown previously that the mouse peripherin splice variant, Per 61, when expressed in motor neurons can cause them to degenerate (Robertson et al JCB, 2003) and that deregulated peripherin and NFL splice variant expression can cause inclusion formation (Robertson et al, 2003 and the present results).

Nucleic acid sequences having substantial homology include nucleic acid sequences having at least about 50 percent identity with a protein encoded by SEQ ID NO:2, 27, 7 or 9, respectively, or the full-length anti-sense sequence thereto. The level of homology, according to various aspects of the disclosure is at least about 60 percent; at least about 63 percent; at least about 65 percent; at least about 68 percent; at least about 70 percent; at least about 73 percent; at least about 75 percent; at least about 78 percent; at least about 80 percent; at least about 83 percent; at least about 85 percent; at least about 88 percent; at least about 90 percent; at least about 93 percent; at least about 95 percent; or at least about 98 percent. Methods for aligning the sequences to be compared and determining the level of homology between the sequences are described in detail above.

In one aspect, the homologous nucleic acid molecule of the disclosure described above has a homology of at least about 50, at least about 55, at least about 60 percent; at least about 63 percent; at least about 65 percent; at least about 68 percent; at least about 70 percent; at least about 73 percent; at least about 75 percent; at least about 78 percent; at least about 80 percent; at least 80 percent; at least about 83 percent; at least about 85 percent; at least about 88 percent; at least about 90 percent; at least about 93 percent; at least about 95 percent; or at least about 98 percent within the sequences retained from intron 3 (nucleic acids 1328 to 1688 of SEQ ID NO:1 numbering from the first ATG of the human peripherin gene sequence) and/or intron 4 (nucleic acids 1857 to 1947 of SEQ ID NO:1 numbering from the first ATG of the human peripherin gene sequence); or within the sequences encoding SEQ ID NO:8 and SEQ ID NO:10, the unique sequences generated from the joining of the 5′ splice sites with the 3′ donor sites for NFL-60 and NFL-57 respectively.

The disclosure is not to be restricted by this homology, for instance, nucleic acid sequences having at least a 50% homology with the sequence shown in SEQ ID NO:2, 27, 7 or 9 are also encompassed within the scope of the present disclosure where there is at least 50%, at least 60%, at least 70%, at least 80%, at least 90%, at least 95% or at least 98% homology with the sequence from nucleic acids 703 to 1063 (intron 3); or 1232 to 1322 (intron 4) of the cDNA sequence as shown in SEQ ID NO:2; or 241 to 270 of the cDNA sequence as shown in SEQ ID NO:7; or 583 to 612 of the cDNA sequence as shown in SEQ ID NO:9.

Sequence identity can be calculated according to methods known in the art. Sequence identity is most preferably assessed by the algorithm of BLAST version 2.1 advanced search. BLAST is a series of programs that are available online at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

The term “sequence that hybridizes” means a nucleic acid sequence that can hybridize to a sequence of (a), (b), (c) or (d) under stringent hybridization conditions. Appropriate stringency conditions which promote nucleic acid hybridization are known to those skilled in the art, or may be found in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.−16.6(Log 10[Na+])+0.41(%(G+C)−600/l). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5×sodium chloride/sodium citrate (SSC)/5×Denhardt's solution/1.0% SDS at Tm (based on the above equation)−5° C., followed by a wash of 0.2×SSC/0.1% SDS at 60° C.

Isolated nucleic acid molecules having sequences which differ from the nucleic acid sequence shown in SEQ ID NO: 2, 27, 7 or 9 due to degeneracy in the genetic code are also within the scope of the disclosure. Such nucleic acids encode functionally equivalent proteins but differ in sequence from the above mentioned sequences due to degeneracy in the genetic code.

An isolated nucleic acid molecule of the disclosure which comprises DNA can be isolated by preparing a labelled nucleic acid probe based on all or part of the nucleic acid sequences as shown in SEQ ID NO: 2, 27, 7 or 9 and using this labelled nucleic acid probe to screen an appropriate DNA library (e.g. a cDNA or genomic DNA library).

An isolated nucleic acid molecule of the disclosure which is DNA can also be isolated by selectively amplifying a nucleic acid encoding a novel protein of the disclosure using the polymerase chain reaction (PCR) methods and cDNA or genomic DNA. It is possible to design synthetic oligonucleotide primers from the nucleic acid sequence as shown in SEQ ID NO: 2, 27, 7 or 9 for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al., Biochemistry, 18, 5294 5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the disclosure which is RNA can be isolated by cloning a cDNA encoding a novel protein of the disclosure into an appropriate vector which allows for transcription of the cDNA to produce an RNA molecule which encodes a protein of the disclosure. For example, a cDNA can be cloned downstream of a bacteriophage promoter, (e.g., a T7 promoter) in a vector, cDNA can be transcribed in vitro with T7 polymerase, and the resultant RNA can be isolated by standard techniques.

A nucleic acid molecule of the disclosure may also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

The sequence of a nucleic acid molecule of the disclosure may be inverted relative to its normal presentation for transcription to produce an antisense nucleic acid molecule. The term “antisense” nucleic acid molecule is a nucleotide sequence that is complementary to its target. Preferably, an antisense sequence is constructed by inverting a region preceding or targeting the initiation codon or an unconserved region. In another embodiment the antisense sequence targets all or part of the mRNA or cDNA of Per 28, NFL-60 or NFL-57. In particular, the nucleic acid sequences contained in the nucleic acid molecules of the disclosure or a fragment thereof may be inverted relative to its normal presentation for transcription to produce antisense nucleic acid molecules. In one embodiment the antisense molecules can be used to inhibit Per 28 expression, Per 32 expression, NFL-60 expression or NFL-57 expression.

The antisense nucleic acid molecules of the disclosure or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, the disclosure provides siRNA that target Per 3,4 as shown in SEQ ID NO:2; NFL-60 as shown in SEQ ID NO:7 or NFL-57 as shown in SEQ ID NO:9. Accordingly, in one aspect, the disclosure provides the forward and reverse primers as shown in SEQ ID NOs:19-22 for interference with Per3,4. In another aspect, the disclosure provides the forward and reverse primers as shown in SEQ ID NOs: 23 and 24 for interference with NFL-60 and the forward and reverse primers as shown in SEQ ID NOs: 25 and 26 for interference with NFL-57.

The disclosure also provides nucleic acids encoding fusion proteins comprising a novel protein of the disclosure and a selected protein, or a selectable marker protein (see below).

### (B) Proteins/Polypeptides

The present application further contemplates an isolated Per 28 protein, an isolated Per 32 protein, an isolated NFL-60 protein and an isolated NFL-57 protein. In an embodiment of the disclosure, an isolated protein is provided which has the amino acid sequence as shown in SEQ ID NO:5 or a fragment, having the retained first 10 amino acids encoded by intron 3, thereof. The present disclosure also encompasses peptides encoded by the nucleic acid sequence of SEQ ID NO:2. In another embodiment, an isolated protein is provided which has the amino acid sequence as shown in SEQ ID NO:28 or a fragment, having the retained amino acids encoded by intron 4, thereof. The present disclosure also encompasses peptides encoded by the nucleic acid sequence of SEQ ID NO:27. In another embodiment of the disclosure, an isolated protein is provided which has the amino acid sequence as shown in SEQ ID NO:7 or a fragment, having the exon 1 amino acid sequence of NFL-60, thereof. In yet another embodiment of the disclosure, an isolated protein is provided which has the amino acid sequence as shown in SEQ ID NO:9 or a fragment, having the exon 1 amino acid sequence of NFL-57, thereof.

Within the context of the present application, a protein of the disclosure may in one embodiment include various structural forms of the primary protein. For example, a protein of the disclosure may be in the form of acidic or basic salts or in neutral form. In addition, individual amino acid residues may be modified by oxidation or reduction.

In addition to the full-length amino acid sequence (SEQ ID NO: 5, 28, 7 or 9), the proteins of the present disclosure may also include truncations of the proteins, and analogs, and homologs of the proteins and truncations thereof as described herein. Truncated proteins may comprise peptides of at least 10 and preferably at least fourteen amino acid residues. For example, the unique sequence in Per 28 is 10 amino acids long, the unique sequence in the intron 4 retained peripherin protein is 21 amino acids long, and NFL-60 and NFL-57 are generated by splicing deletions, thus a new sequence is generated by the joining together of the 5′ and 3′ cleavage sites. The unique sequences generated at the nucleic acid and amino acid levels are underlined in SEQ ID 7 and 9.

In an embodiment, the disclosure provides a peptide fragment comprising amino acids 235 to 244 of SEQ ID NO:5 or an analog or homolog thereof. In one embodiment, the disclosure provides a peptide fragment consisting essentially of the amino acid sequence VSGPGIRGGF (SEQ ID NO:4) or an analog or homolog thereof.

In another embodiment, the disclosure provides a peptide fragment comprising amino acids 291 to 311 of SEQ ID NO:28 or an analog or homolog thereof. In one embodiment, the disclosure provides a peptide fragment consisting essentially of the amino acid sequence VQEPGGPARRDAGVVSRVPAD (SEQ ID NO:29) or an analog or homolog thereof.

In another embodiment, the disclosure provides a peptide fragment comprising amino acids 81 to 90 of SEQ ID NO:7 or an analog or homolog thereof. In one embodiment, the disclosure provides a peptide fragment consisting essentially of the amino acid sequence NDLKSIRDLR (SEQ ID NO: 8) or an analog or homolog thereof.

In yet another embodiment, the disclosure provides a peptide fragment comprising amino acids 195 to 204 of SEQ ID NO:9 or an analog or homolog thereof. In one embodiment, the disclosure provides a peptide fragment having the amino acid sequence ARKGAKNTDA (SEQ ID NO: 10) or an analog or homolog thereof.

Analogs of the proteins having the amino acid sequences shown in SEQ ID NO: 5, 28, 7 or 9 as described herein, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the disclosure with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

Without the intention of being limited thereby, in one embodiment, the substitutions of amino acids are made that preserve the structure responsible for the ability to associate with aggregated and inclusion bodies of the proteins disclosed herein. Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. These sorts of substitutions and interchanges are well known to those skilled in the art. Other substitutions might well be possible. Of course, it would also be expected that the greater the percentage of homology, i.e., sequence similarity, of a variant protein with a naturally occurring protein, the greater the retention of its activity. Of course, as protein variants having the activity of Per 28, NFL-60 or NFL-57 as described herein are intended to be within the scope of this disclosure, so are nucleic acids encoding such variants.

One or more amino acid insertions may be introduced into the amino acid sequences shown in SEQ ID NO: 5, 28, 7 or 9. Amino acid insertions may consist of single amino acid residues or sequential amino acids ranging from 2 to 15 amino acids in length. For example, amino acid insertions may be used to destroy target sequences so that the protein is no longer involved in aggregated or inclusion bodies. This procedure may be used in vivo to inhibit the activity of Per 28, NFL-60 or NFL-57, respectively.

Deletions may consist of the removal of one or more amino acids, or discrete portions: from the amino acid sequence shown in SEQ ID NO:5, excluding the region encoded by introns 3 and/or 4 (amino acids 235 to 244 of SEQ ID NO:5 or amino acids 291 to 311 of SEQ ID NO:28); from the amino acid sequence shown in SEQ ID NO: 7, excluding the region encoded amino acids 81 to 90 of SEQ ID NO:7; or from the amino acid sequence shown in SEQ ID NO:9, excluding the region encoded by amino acids 195 to 204 of SEQ ID NO:9. The deleted amino acids may or may not be contiguous. The lower limit length of the resulting analog with a deletion mutation is about 10 amino acids, preferably 100 amino acids.

Analogs of the proteins of the disclosure may be prepared by introducing mutations in the nucleotide sequence encoding the protein. Mutations in nucleotide sequences constructed for expression of analogs of a protein of the disclosure must preserve the reading frame of the coding sequences. Furthermore, the mutations will preferably not create complementary regions that could hybridize to produce secondary mRNA structures, such as loops or hairpins, which could adversely affect translation of the mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a protein of the disclosure may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al (Molecular Cloning: A Laboratory Manual, 2nd Ed., Cold Spring Harbor Laboratory Press, 1989).

The proteins of the disclosure also include homologs of the amino acid sequence shown in SEQ ID NO: 5, 28, 7, 9 and/or truncations thereof as described herein. Such homologs are proteins whose amino acid sequences are encoded by nucleic acid sequences that hybridize under stringent hybridization conditions (see discussion of stringent hybridization conditions herein) with a probe used to obtain a protein of the disclosure. Homologs of a protein of the disclosure will have the same regions which are characteristic of the protein.

A homologous protein includes a protein with an amino acid sequence having at least 60%, at least 70%, at least 80%, at least 90%, at least 95% or at least 98% identity with the amino acid sequence as shown in SEQ ID NO: 5, 28, 7 or 9.

The application also contemplates isoforms of the proteins of the disclosure. An isoform contains the same number and kinds of amino acids as a protein of the disclosure, but the isoform has a different molecular structure. The isoforms contemplated by the present disclosure are those having the same properties as a protein of the disclosure as described herein.

The present application also includes a protein described above conjugated with a selected protein, or a selectable marker protein (see below) to produce fusion proteins. Additionally, immunogenic portions of a protein described above are within the scope of the application. The immunogenic portion of a protein is that portion that if administered to a patient can induce an immune response and preferably an antibody response.

A further advantage may be obtained through chimeric forms of the protein, as known in the art. A DNA sequence encoding the entire protein, or a portion of the protein, could thus be linked, for example, with a sequence coding for the C-terminal portion of E. coli β-galactosidase to produce a fusion protein.

The proteins described above (including truncations, analogs, etc.) may be prepared using recombinant DNA methods. These proteins may be purified and/or isolated to various degrees using techniques known in the art. Accordingly, nucleic acid molecules of the present disclosure having a sequence which encodes a protein of the disclosure may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule of the disclosure and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The disclosure therefore contemplates a recombinant expression vector of the disclosure containing a nucleic acid molecule of the disclosure, or a fragment thereof, and the necessary regulatory sequences for the transcription and translation of the inserted protein-sequence. Suitable regulatory sequences may be derived from a variety of sources, including bacterial, fungal, or viral genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

The disclosure further provides a recombinant expression vector comprising a DNA nucleic acid molecule of the disclosure cloned into the expression vector in an antisense orientation. That is, the DNA molecule is operatively linked to a regulatory sequence in a manner which allows for expression, by transcription of the DNA molecule, of an RNA molecule which is antisense to a nucleotide sequence of the disclosure preferably comprising the nucleotides as shown in SEQ ID NO: 2, 27, 7, 9 or fragments thereof. Regulatory sequences operatively linked to the antisense nucleic acid can be chosen which direct the continuous expression of the antisense RNA molecule.

The recombinant expression vectors of the disclosure may also contain a selectable marker gene that facilitates the selection of host cells transformed or transfected with a recombinant molecule of the disclosure. Examples of selectable marker genes are genes encoding a protein which confers resistance to certain drugs, such as G418 and hygromycin. Examples of other markers which can be used are: green fluorescent protein (GFP), β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the disclosure and in particular to determine the effect of a mutation on expression and phenotype. It will be appreciated that selectable markers can be introduced on a separate vector from the nucleic acid of interest.

The recombinant expression or cloning vectors of the disclosure may also contain genes which encode a fusion moiety which provides increased expression of the recombinant protein; increased solubility of the recombinant protein; and aid in the purification of a target recombinant protein by acting as a ligand in affinity purification. For example, a proteolytic cleavage site may be added to the target recombinant protein to allow separation of the recombinant protein from the fusion moiety subsequent to purification of the fusion protein.

Recombinant expression vectors can be introduced into host cells to produce a transformed host cell. The term “transformed host cell” is intended to include prokaryotic and eukaryotic cells which have been transformed or transfected with a recombinant expression vector of the disclosure. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium chloride mediated transformation. Nucleic acid can be introduced into mammalian cells via conventional techniques such as calcium phosphate or calcium chloride co precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other such laboratory textbooks.

Suitable host cells include a wide variety of prokaryotic and eukaryotic host cells. For example, the proteins of the disclosure may be expressed in bacterial cells such as E. coli, insect cells (using baculovirus), yeast cells or mammalian cells, COS1 cells. Other suitable host cells can be found in Goeddel, Gene Expression Technology: Methods in Enzymology 185, Academic Press, San Diego, Calif. (1991).

The disclosure includes a microbial cell that contains and is capable of expressing a heterologous a nucleic acid molecule having a nucleotide sequence as encompassed by the disclosure. The heterologous nucleic acid molecule can be DNA.

Isolated DNA of the disclosure can be contained in a recombinant cloning vector.

The disclosure includes a stably transfected cell line which expresses any one or more proteins as defined by the disclosure.

The disclosure includes a culture of cells transformed with a recombinant DNA molecule having a nucleotide sequence as encompassed by the disclosure. Such a culture of cells can be eukaryotic. They can be piscine—particularly zebrafish, mammalian—particularly human, rat or mouse, for example.

The application also contemplates a process for producing any protein as defined by the disclosure. The process includes such steps as:

preparing a DNA fragment including a nucleotide sequence which encodes said protein;

incorporating the DNA fragment into an expression vector to obtain a recombinant DNA molecule which includes the DNA fragment and is capable of undergoing replication;

transforming a host cell with said recombinant DNA molecule to produce a transformant which can express said protein;

culturing the transformant to produce said protein; and

recovering said protein from resulting cultured mixture.

More particularly, the application provides a method of preparing a purified protein of the disclosure comprising introducing into a host cell a recombinant nucleic acid encoding the protein, allowing the protein to be expressed in the host cell and isolating and purifying the protein. Preferably, the recombinant nucleic acid is a recombinant expression vector. Proteins can be isolated from a host cell expressing the protein and purified according to standard procedures of the art, including ammonium sulfate precipitation, column chromatography (e.g. ion exchange, gel filtration, affinity chromatography, etc.), electrophoresis, and ultimately, crystallization [see generally, “Enzyme Purification and Related Techniques”, Methods in Enzymology, 22, 233-577 (1971)].

Alternatively, the protein or parts thereof can be prepared by chemical synthesis using techniques well known in the chemistry of proteins such as solid phase synthesis [Merrifield 1964] or synthesis in homogeneous solution [Houbenwycl, 1987].

### (C) Binding Proteins

The application provides binding proteins that bind to Per 28, but do not bind to wild-type peripherin. The application also provides binding proteins that bind to the Per 32 protein, but do not bind to wild-type peripherin. The application also provides binding proteins that bind to NFL-60, but do not bind to wild type NFL. The application further provides binding proteins that bind to NFL-57, but do not bind to wild type NFL.

The term “binding protein” as used herein refers to proteins that specifically bind to another substance. In an embodiment, the binding proteins can bind to Per 28 but not wild type peripherin. Accordingly, in one embodiment, the binding protein binds to the protein having the amino acid sequence shown in SEQ ID NO:5. In another embodiment, the binding protein binds to a protein encoded by the nucleic acid sequence as shown in SEQ ID NO:2. In another embodiment, the binding proteins can bind to the Per 32 protein but not wild type peripherin. Accordingly, in one embodiment, the binding protein binds to the protein having the amino acid sequence shown in SEQ ID NO:28. In another embodiment, the binding protein binds to a protein encoded by the nucleic acid sequence as shown in SEQ ID NO:27. In another embodiment, the binding proteins can bind to NFL-60 but not wild type NFL. Accordingly, in one embodiment, the binding protein binds to the protein having the amino acid sequence shown in SEQ ID NO:7. In another embodiment, the binding protein binds to a protein encoded by the nucleic acid sequence as shown in SEQ ID NO:7. In yet another embodiment, the binding proteins can bind to NFL-57 but not wild type NFL. Accordingly, in one embodiment, the binding protein binds to the protein having the amino acid sequence shown in SEQ ID NO:9. In another embodiment, the binding protein binds to a protein encoded by the nucleic acid sequence as shown in SEQ ID NO:9. In another embodiment, the binding proteins are antibodies or antibody fragments thereof. In a further embodiment, the binding proteins are monoclonal antibodies or fragments thereof.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

The disclosure also contemplates the use of “peptide mimetics” for the binding proteins. Peptide mimetics are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of the binding proteins of the disclosure, such as its ability to bind to misfolded or monomeric SOD1. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to the binding proteins of the disclosure.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

In one embodiment of the disclosure, the binding protein binds to an epitope on the Per 28 protein comprising the portion that is encoded by intron 3 (nucleic acids 1328-1688 of SEQ ID NO:1 and nucleic acids 703 to 1063 of SEQ ID NO:2) which is normally not found in the wild type peripherin protein. For example, in one embodiment of the disclosure the epitope consists essentially of the amino acid sequence as shown in SEQ ID NO:4. Per 3,4 itself is alternatively spliced, such that there is an mRNA corresponding to intron 4 retention. Accordingly, in another embodiment, the binding protein binds to an epitope on the retained intron 4 peripherin protein comprising the portion that is encoded by intron 4 (nucleic acids 1232 to 1322 of SEQ ID NO:2 and nucleic acids 1857 to 1947 of SEQ ID NO:1. For example, in one embodiment of the disclosure the epitope consists essentially of the amino acid sequence as shown in SEQ ID NO:29.

In another embodiment of the disclosure, the binding protein binds to an epitope on the NFL-60 protein comprising the portion of exon 1 that is encoded by nucleic acids 241 to 269 of SEQ ID NO:7, which is normally not found in the wild type NFL protein. For example, in one embodiment of the disclosure the epitope consists essentially of the amino acid sequence as shown in SEQ ID NO:8.

In yet another embodiment of the disclosure, the binding protein binds to an epitope on the NFL-57 protein comprising the portion of exon 1 that is encoded by nucleic acids 583 to 612 of SEQ ID NO:9, which is normally not found in the wild type NFL protein. For example, in one embodiment of the disclosure the epitope comprises the amino acid sequence as shown in SEQ ID NO:10.

The term “epitope” as used herein refers to the part of the protein which contacts the antigen binding site of the binding protein of the disclosure.

In one embodiment, the binding protein is an antibody that binds to the epitope comprising the amino acid sequence as shown in SEQ ID NO:4, 29, 8 or 10.

The fragments of Per 28 described above are useful as antigens in preparing antibodies that bind to the protein portion encoded by intron 3 of Per 28 but do not bind to the wild type peripherin. In a particular embodiment, the antigen comprises the unique sequence in Per 28 (as shown in SEQ ID NO:4), which is encoded by the first 30 bp of intron 3 as shown in SEQ ID NO:3, wherein a stop codon is then reached. The fragments of Per 32 described above are useful as antigens in preparing antibodies that bind to the protein portion encoded by intron 4 but do not bind to the wild type peripherin. In a particular embodiment, the antigen comprises the unique sequence in Per 32 (as shown in SEQ ID NO:29). The fragments of NFL-60 described above are useful as antigens in preparing antibodies that bind to the protein portion encoded by 241 to 269 of SEQ ID NO:7 of NFL-60 but do not bind to the wild type NFL. The fragments of NFL-57 described above are useful as antigens in preparing antibodies that bind to the protein portion encoded by nucleic acids 583 to 612 of SEQ ID NO:9 of NFL-57 but do not bind to the wild type NFL.

In an embodiment, the disclosure provides a method of making an antibody that binds to Per 28 comprising the following steps:
a) immunizing a host with an immunogen comprising an isolated Per 28 fragment having the amino acid sequence as shown in SEQ ID NO:4;
b) isolating an antibody from said host that binds Per 28.

In an embodiment, the disclosure provides a method of making an antibody that binds to Per 32 comprising the following steps:
a) immunizing a host with an immunogen comprising an isolated intron 4 retained peripherin protein fragment having the amino acid sequence as shown in SEQ ID NO:29;
b) isolating an antibody from said host that binds Per 32.

In another embodiment, the disclosure provides a method of making an antibody that binds to NFL-60 comprising the following steps:
a) immunizing a host with an immunogen comprising an isolated NFL-60 fragment having the amino acid sequence as shown in SEQ ID NO:8;
b) isolating an antibody from said host that binds NFL-60.

In yet another embodiment, the disclosure provides a method of making an antibody that binds to NFL-57 comprising the following steps:
a) immunizing a host with an immunogen comprising an isolated NFL-57 fragment having the amino acid sequence as shown in SEQ ID NO:10;
b) isolating an antibody from said host that binds NFL-57.

Conventional methods can be used to prepare the antibodies. For example, by using a peptide of a protein of the disclosure, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)); the EBV-hybridoma technique to produce human monoclonal antibodies (Cole et al. Monoclonal Antibodies in Cancer Therapy (1985) Allen R. Bliss, Inc., pages 77-96); and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the disclosure also contemplates hybridoma cells secreting monoclonal antibodies with specificity for a protein of the disclosure.

Chimeric antibody derivatives, i.e., antibody molecules that combine a non-human animal variable region and a human constant region are also contemplated within the scope of the disclosure. Chimeric antibody molecules can include, for example, the antigen binding domain from an antibody of a mouse, rat, or other species, with human constant regions. Conventional methods may be used to make chimeric antibodies containing the immunoglobulin variable region which recognizes a Per 28, NFL-60 or NFL-57 protein of the disclosure (See, for example, Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a protein of the disclosure as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982); and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments reactive against a protein of the disclosure may also be generated by screening expression libraries encoding immunoglobulin genes, or portions thereof, expressed in bacteria with peptides produced from nucleic acid molecules of the present disclosure. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)).

Binding proteins may be useful for antagonizing the effects of the splice variants. Alternatively the splicing events that generate these variants could be prevented using Spliceosome-mediated RNA trans-splicing or SMaRT™ technology (Intronn Inc).

### (D) Diagnostic Applications

The above nucleic acid and peptide molecules of the disclosure can be used to diagnose, detect and monitor amyotrophic lateral sclerosis (ALS). Determination of peptide or nucleic acid expression levels could assist not only in identifying a medical condition but in determining the appropriate course of treatment.

### (i) Nucleic Acids

The above described nucleic acid molecules allow those skilled in the art to construct nucleotide probes for use in the detection of nucleotide sequences homologous to the nucleotide sequences encoding Per 28, Per 32, NFL-60, NFL-57 or a fragment thereof in a sample.

Accordingly, in an embodiment of the disclosure, the inventors provide a method of detecting or monitoring ALS in a subject having or suspected of having ALS, comprising detecting the presence or a change in the amount of a nucleotide encoding Per 28, Per 32, NFL-60 or NFL-57 in a sample, wherein the presence of a nucleic acid encoding Per 28, Per 32, NFL-60 or NFL-57 is indicative of ALS.

In one embodiment, a method of detecting the presence or a change in the amount of nucleic acid molecules encoding a Per 28, Per 32, NFL-60 or NFL-57 protein in a sample comprises contacting the sample under hybridization conditions with one or more nucleotide probes which hybridize to the nucleic acid molecules and are labelled with a detectable marker, and, determining the degree of hybridization between the nucleic acid molecule in the sample and the nucleotide probe(s).

A nucleotide probe may be labelled with a detectable marker such as a radioactive label which provides for an adequate signal and has sufficient half life such as 32P, 3H, 14C or the like. Other detectable markers which may be used include antigens that are recognized by a specific labelled antibody, fluorescent compounds, enzymes, antibodies specific for a labelled antigen, and chemiluminescent compounds. An appropriate label may be selected having regard to the rate of hybridization and binding of the probe to the nucleotide to be detected and the amount of nucleotide available for hybridization.

Hybridization conditions which may be used in methods of the disclosure are known in the art and are described for example in Sambrook J, Fritch E F, Maniatis T. In: Molecular Cloning, A Laboratory Manual, 1989. (Nolan C, Ed.), Cold Spring Harbor Laboratory Press, Cold Spring Harbor, N.Y. The hybridization product may be assayed using techniques known in the art. The nucleotide probe may be labelled with a detectable marker as described herein and the hybridization product may be assayed by detecting the detectable marker or the detectable change produced by the detectable marker.

A nucleic acid molecule of the disclosure also permits the identification and isolation, or synthesis of nucleotide sequences which may be used as primers to amplify a nucleic acid molecule of the disclosure, for example, in a polymerase chain reaction (PCR) which is discussed in more detail below. Examples of primers are shown in Table 9 and below.

The length and bases of primers for use in a PCR are selected so that they will hybridize to different strands of the desired sequence and at relative positions along the sequence such that an extension product synthesized from one primer when it is separated from its template can serve as a template for extension of the other primer into a nucleic acid of defined length. Primers which may be used in the disclosure are oligonucleotides, i.e., molecules containing two or more deoxyribonucleotides of the nucleic acid molecule of the disclosure which occur naturally as in a purified restriction endonuclease digest or are produced synthetically using techniques known in the art such as for example phosphotriester and phosphodiester methods (See Good et al. Nucl. Acid Res 4:2157, 1977) or automated techniques (See for example, Conolly, B. A. Nucleic Acids Res. 15:15(7): 3131, 1987). The primers are capable of acting as a point of initiation of synthesis when placed under conditions which permit the synthesis of a primer extension product which is complementary to a DNA sequence of the disclosure, i.e., in the presence of nucleotide substrates, an agent for polymerization such as DNA polymerase and at suitable temperature and pH. Preferably, the primers are sequences that do not form secondary structures by base pairing with other copies of the primer or sequences that form a hair pin configuration. The primer preferably contains between about 7 and 25 nucleotides.

The primers may be labelled with detectable markers which allow for detection of the amplified products. Suitable detectable markers are radioactive markers such as P-32, S-35, I-125, and H-3, luminescent markers such as chemiluminescent markers, preferably luminol, and fluorescent markers, preferably dansyl chloride, fluorcein-5-isothiocyanate, and 4-fluor-7-nitrobenz-2-axa-1,3 diazole, enzyme markers such as horseradish peroxidase, alkaline phosphatase, β-galactosidase, acetylcholinesterase, or biotin.

It will be appreciated that the primers may contain non-complementary sequences provided that a sufficient amount of the primer contains a sequence which is complementary to a nucleic acid molecule of the disclosure or oligonucleotide fragment thereof, which is to be amplified. Restriction site linkers may also be incorporated into the primers allowing for digestion of the amplified products with the appropriate restriction enzymes facilitating cloning and sequencing of the amplified product.

In an embodiment of the disclosure a method of determining the presence of a nucleic acid molecule having a sequence encoding a protein of the disclosure is provided comprising treating the sample with primers which are capable of amplifying the nucleic acid molecule or a predetermined oligonucleotide fragment thereof in a polymerase chain reaction to form amplified sequences, under conditions which permit the formation of amplified sequences and, assaying for amplified sequences.

Polymerase chain reaction as used herein refers to a process for amplifying a target nucleic acid sequence as generally described in Innis et al, Academic Press, 1990 in Mullis el al., U.S. Pat. No. 4,863,195 and Mullis, U.S. Pat. No. 4,683,202. Conditions for amplifying a nucleic acid template are described in M. A. Innis and D. H. Gelfand, PCR Protocols, A Guide to Methods and Applications M. A. Innis, D. H. Gelfand, J. J. Sninsky and T. J. White eds, pp 3-12, Academic Press 1989.

The amplified products can be isolated and distinguished based on their respective sizes using techniques known in the art. For example, after amplification, a DNA sample can be separated on an agarose gel and visualized, after staining with ethidium bromide, under ultra violet (uv) light. DNA may be amplified to a desired level and a further extension reaction may be performed to incorporate nucleotide derivatives having detectable markers such as radioactive labelled or biotin labelled nucleoside triphosphates. The primers may also be labelled with detectable markers as discussed above. The detectable markers may be analyzed by restriction enzyme digestion and electrophoretic separation or other techniques known in the art.

Conditions which may be employed in the methods of the disclosure using PCR are those which permit hybridization and amplification reactions to proceed in the presence of DNA in a sample and appropriate complementary hybridization primers. Conditions suitable for a polymerase chain reaction are generally known in the art. For example, see M. A. Innis and D. H. Gelfand, PCR Protocols, A guide to Methods and Applications M. A. Innis, D. H. Gelfand, J. J. Sninsky and T. J. White eds, pp 3-12, Academic Press 1989. To amplify DNA template strands, preferably, the PCR utilizes polymerase obtained from the thermophilic bacterium Thermus aquatics (Taq polymerase, GeneAmp Kit, Perkin Elmer Cetus) or other thermostable polymerase.

### (ii) Binding Proteins

In another embodiment, the disclosure provides a method of detecting, monitoring or diagnosing ALS in a subject comprising the steps of:
(a) contacting a sample of said subject with a binding protein specific for Per 28, Per 32, NFL-60 or NFL-57;
(b) measuring the amount of the binding protein-protein complex in the sample; and
(c) comparing the amount of binding protein-protein complex in the sample to a control;

wherein a change in the amount of binding protein-protein complex in the sample as compared to control is indicative of ALS.

In one embodiment, the amount of binding protein-protein complex in the sample is increased compared to control. In another embodiment, the amount of binding protein-protein complex in the sample is decreased compared to control.

The term “control” as used herein refers to a sample from an individual or a group of subjects who as not having ALS.

The term “subject” as used herein refers to any member of the animal kingdom, preferably a human being.

The term “sample” as used herein refers to any fluid, cell or tissue sample from a subject which can be assayed for Per 28, Per 32, NFL-60 or NFL-57. In one embodiment, the sample comprises, without limitation, cerebrospinal fluid, plasma, blood serum, whole blood, spinal cord tissue, brain cells, motor neurons, a portion of the dorsal horn, urine or peripheral blood cells.

In one embodiment, the binding protein used in the above method is an antibody that binds an antigen to form an antibody-antigen complex. The antibodies may be labelled with a detectable marker including various enzymes, fluorescent materials, luminescent materials and radioactive materials. Examples of suitable enzymes include horseradish peroxidase, alkaline phosphatase, β-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include S-35, Cu-64, Ga-67, Zr-89, Ru-97, Tc-99m, Rh-105, Pd-109, In-111, I-123, I-125, I-131, Re-186, Au-198, Au-199, Pb-203, At-211, Pb-212 and Bi-212. The antibodies may also be labelled or conjugated to one partner of a ligand binding pair. Representative examples include avidin-biotin and riboflavin-riboflavin binding protein. Methods for conjugating or labelling the antibodies discussed above with the representative labels set forth above may be readily accomplished using conventional techniques.

Antibodies reactive against Per 28, Per 32, NFL-60 or NFL-57 proteins described above (e.g., enzyme conjugates or labelled derivatives) may be used to detect a Per 28, Per 32, NFL-60 or NFL-57 protein, respectively, in various samples, for example they may be used in any known immunoassays which rely on the binding interaction between an antigenic determinant of a protein of the disclosure and the antibodies. Examples of such assays are radioimmunoassays, western immunoblotting, enzyme immunoassays (e.g., ELISA), immunofluorescence, immunoprecipitation, latex agglutination, hemagglutination, and histochemical tests. Thus, the antibodies may be used to identify or quantify the amount of a protein in a sample.

A sample may be tested for the presence or absence of a Per 28 protein by contacting the sample with an antibody specific for an epitope of a Per 28 protein which antibody is capable of being detected after it becomes bound to a Per 28 protein in the sample, and assaying for antibody bound to a Per 28 protein in the sample, or unreacted antibody.

A sample may be tested for the presence or absence of a retained intron 4 peripherin protein by contacting the sample with an antibody specific for an epitope of a Per 32 protein which antibody is capable of being detected after it becomes bound to a retained intron 4 peripherin protein in the sample, and assaying for antibody bound to a Per 32 protein in the sample, or unreacted antibody.

A sample may be tested for the presence or absence of a NFL-60 protein by contacting the sample with an antibody specific for an epitope of a NFL-60 protein which antibody is capable of being detected after it becomes bound to a NFL-60 protein in the sample, and assaying for antibody bound to a NFL-60 protein in the sample, or unreacted antibody.

A sample may be tested for the presence or absence of a NFL-57 protein by contacting the sample with an antibody specific for an epitope of a NFL-57 protein which antibody is capable of being detected after it becomes bound to a NFL-57 protein in the sample, and assaying for antibody bound to a NFL-57 protein in the sample, or unreacted antibody.

In a method of the application, a predetermined amount of a sample or concentrated sample is mixed with antibody or labelled antibody. The amount of antibody used in the method is dependent upon the labelling agent chosen. The resulting protein bound to antibody or labelled antibody may be isolated by conventional isolation techniques, for example, salting out, chromatography, electrophoresis, gel filtration, fractionation, absorption, polyacrylamide gel electrophoresis, agglutination, or combinations thereof.

The sample or antibody may be insolubilized, for example, the sample or antibody can be reacted using known methods with a suitable carrier. Examples of suitable carriers are Sepharose or agarose beads. When an insolubilized sample or antibody is used protein bound to antibody or unreacted antibody is isolated by washing. For example, when the sample is blotted onto a nitrocellulose membrane, the antibody bound to a protein of the disclosure is separated from the unreacted antibody by washing with a buffer, for example, phosphate buffered saline (PBS) with bovine serum albumin (BSA).

When labelled antibody is used, the presence of a Per 28, Per 3, NFL-60 or NFL-57 protein can be determined by measuring the amount of labelled antibody bound to a protein of the disclosure in the sample or of the unreacted labelled antibody. The appropriate method of measuring the labelled material is dependent upon the labelling agent.

When unlabelled antibody is used in a method, the presence of a Per 28, Per 32, NFL-60 or NFL-57 can be determined by measuring the amount of antibody bound to the Per 28, Per 32, NFL-60 or NFL-57, respectively, using substances that interact specifically with the antibody to cause agglutination or precipitation. In particular, labelled antibody against an antibody specific for a protein, can be added to the reaction mixture. The antibody against an antibody specific for a protein of the disclosure can be prepared and labelled by conventional procedures known in the art which have been described herein. The antibody against an antibody specific for a protein of the disclosure may be a species specific anti-immunoglobulin antibody or monoclonal antibody, for example, goat anti-rabbit antibody may be used to detect rabbit antibody specific for a protein of the disclosure.

The Per 28, Per 32, NFL-60 and NFL-57 unique sequences are useful for testing for autoantibodies in CSF, blood and urine. With the production of peripherin and neurofilament splice variants in ALS, generation of autoantibodies to these variants released into the CSF or other bodily fluid can be tested in an ELISA assay or by western blot using peptides, recombinant proteins or purified proteins of Per 28, Per 32, NFL-60 or NFL-57. Autoantibodies to wild type NFL have been found in the CSF of patients with multiple sclerosis (Ehling et al, Multiple Sclerosis, 10:601-606, 2004) and autoantibodies to the neurofilament heavy subunit have been found in various neurological conditions including amyotrophic lateral sclerosis, Parkinson's disease and Alzheimer's disease (eg Karcher et al, Acta Neuropathol, 72:82-85). Autobodies to wild type peripherin have been found in patients with diabetes (eg Puertas et al, J Immunol, 178; 6533-6539.

Any of the methods of the disclosure to diagnose, detect or monitor ALS can be used in addition or in combination with traditional diagnostic techniques for ALS. ALS is difficult to diagnose because the symptoms are similar to those of other neuromuscular disorders, many of which are treatable. The diagnosis is usually based on a complete neurological examination involving tests of muscle weakness and or stiffness, and clinical tests, including studies of nerve conduction velocity and electromyography. A diagnosis is only absolutely confirmed at autopsy.

According to the Internationally accepted El-Escorial criteria for a diagnosis of ALS:

The diagnosis of Amyotrophic Lateral Sclerosis [ALS] requires:
A—the presence of:
(A:1) evidence of lower motor neuron (LMN) degeneration by clinical, electrophysiological or neuropathologic examination,
(A:2) evidence of upper motor neuron (UMN) degeneration by clinical examination, and
(A:3) progressive spread of symptoms or signs within a region or to other regions,as determined by history or examination, together with
B—the absence of:
(B:1) electrophysiological and pathological evidence of other disease processes that might explain the signs of LMN and/or UMN degeneration, and
(B:2) neuroimaging evidence of other disease processes that might explain the observed clinical and electrophysiological signs.

### (D) Drug Screening

In another embodiment, the disclosure provides a method of identifying substances for the treatment or prevention of ALS comprising the steps of:

(a) contacting a sample from a subject treated with a substance with any one of the binding proteins of the disclosure, wherein binding is indicative of the presence of Per 28, Per 32, NFL-60 or NFL-57 in the sample,

(b) detecting the level of binding in the sample, and

(c) comparing the level of binding in the sample to the level of binding in a control,

wherein an altered level of binding in the sample compared to the control is indicative of a substance for the treatment or prevention of ALS.

A person skilled in the art will appreciate that the control can be a sample from a subject not treated with a substance or treated with a substance that is known not to treat or prevent ALS. Thus, a reduced level of binding in the sample compared to the control is indicative of a substance for the treatment or prevention of amyotrophic lateral sclerosis. In addition, the control can be a sample from the same subject, but before treatment with the substance to be tested or samples from the subject taken at different points of time during treatment with the substance to be tested.

Substances for the treatment or prevention of ALS can also be identified using cells or cell lines. For example, cells or cell lines can be contacted with a substance and then the presence of Per 28, Per 32, NFL-60 or NFL-57 in the cells can be detected using the binding proteins of the disclosure and compared to a control.

A person skilled in the art will appreciate that a library of molecules can be screened by monitoring the effect of the candidate compounds on the amount of Per 28, Per 32, NFL-60 or NFL-57 protein in the sample.

The disclosure also includes the substances identified using the methods of the disclosure, which are useful for the treatment of ALS.

### (E) Kits

A further aspect of the disclosure is a kit for diagnosing, detecting or monitoring ALS comprising any one of the binding proteins of the disclosure and instructions for use. In one embodiment of the disclosure, the binding protein is an antibody. In another embodiment, the binding protein is labeled using a detectable marker.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Peripherin Splice Variant

### Results:

The present inventors have previously shown the expression of a neurotoxic splice variant of peripherin in motor neurons of transgenic mice expressing mutant SOD1 (Robertson et al., 2003). Although overexpression or ablation of peripherin in these mice did not affect disease course (Lariviere et al., 2003), it is now known that motor neuron degeneration caused by mutant SOD1 in transgenic mice is multifactorial, involving both neuronal and non-neuronal components (Clement et al., 2003). Furthermore, mutations in SOD1 are causative of only 1-2% of all FALS whereas peripherin abnormalities are commonly found in both fALS and sALS (He et al., 2004). The neurotoxic splice variant previously described in mouse, Per 61, was generated through the in-frame retention of intron 4, which is 96 bp in length and introduces a 32 amino acid insertion within a highly conserved domain of the peripherin protein (Robertson et al., 2003; Landon et al., 1989; Landon et al., 2000). In humans however, intron 4 is 91 bp in length and its complete retention would introduce a premature stop codon and the generation of a truncated peripherin species. Evidence that such a splicing event could happen in humans came from the identification of an EST sequence in the UCSD Genome Browser retaining part of intron 4 (BE786797). Because abnormal splicing events involving intron 4 of peripherin in mutant SOD1 transgenic mice were found, the present inventors investigated if similar splicing events occurred in ALS. To this end using primer sequences designed to the EST retaining intron 4, a human peripherin transcript retaining both introns 3 and 4 was identified. The identification of this transcript was not due to genomic contamination of our RNA sample as all samples were pre-treated with DNase prior to RT-PCR, controls omitting reverse transcriptase showed no amplification of genomic DNA, oligo (dT)₂₀ was used for first strand synthesis and the use of off-target primers showed the proper splicing of introns 1 and 2. This transcript was in low relative abundance compared to the constitutively spliced transcript and could only be amplified using gene-specific primers within either introns 3 or 4. Intron retention is the least frequent of alternative splicing events in mammals, and as such, is the least studied (Kan et al., 2002; Clark et al., 2002). The retention of intron 3 leads to the introduction of a premature stop codon 30 bp downstream from the start of the intron, generating a truncated protein of ˜28 kDa with a unique 10 amino acid sequence at the C-terminus. Using a specific antisera generated to the unique 10 amino acid sequence, the existence of Per 28 kDa as an alternatively spliced isoform expressed from the Per 3,4 cDNA was established. Per 28 was also expressed from the normal full-length human peripherin gene in SW13vim(−) cells, but at a much lower level. The retention of introns 3 and 4 appeared therefore to induce deregulated peripherin splice variant expression. This included the expression of four other minor species of ˜25 kDa, ˜32 kDa, ˜45 kDa and ˜48 kDa. The 45 kDa species has been positively identified as being derived from the use of an alternate translation start codon within the peripherin transcript. All of these species were also expressed from the human peripherin gene, but at a lower level and at different stoichiometric ratios than from the Per 3,4 cDNA. The deregulated expression of peripherin isoforms from Per 3,4 induced peripherin to form aggregates instead of filaments, raising the possibility that deregulated peripherin splice isoform expression may contribute to peripherin aggregation and inclusion body formation in ALS. This premise was supported by experiments in which the peripherin splicing profile of Per 3,4 was reverted using RNAi to downregulate the expression of Per 28 relative to Per 58, consequently inducing peripherin to form filaments instead of aggregates. We have provided direct evidence that there is a similar deregulaton of peripherin splicing in ALS by showing an upregulation of the Per 3,4 mRNA relative to the constitutively spliced peripherin transcript in ALS lumbar spinal cord compared to controls, and that this corresponded with upregulated expression of Per 28. Interestingly, Per 28 expression was not detected on immunoblots of four control samples, comprising of one non-neurological disease and three neurological disease cases (one multisystems atrophy, two corticobasal degeneration). Although this finding will require more extensive validation it does offer the possibility that Per 28 (and potentially other splice variants) could act as a biomarker. This is especially pertinent, as few investigators study splicing events involving intron retention in mammals (Kan et al., 2002; Clark et al., 2002), and the unique sequences generated by these splicing events may have been overlooked as potential candidates in biomarker studies.

Interestingly, the nuclear DNA/RNA binding protein TDP-43 has recently been identified as a component of ubiquitinated inclusions in ALS (Neumann et al., 2006; Arai et al., 2006). One of the known functions of TDP-43 is to act as a splicing factor (Buratti et al., 2001), and it is therefore tempting to speculate that the deregulated splicing we have observed for peripherin may be causally linked to abnormalities of TDP-43.

### Identification of Peripherin Transcript Retaining Introns 3 and 4

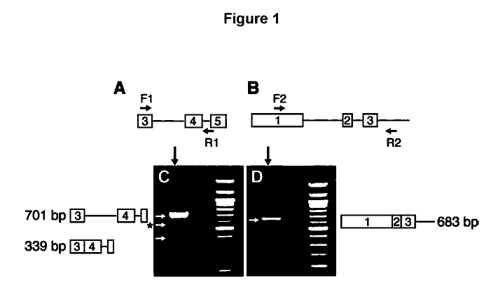

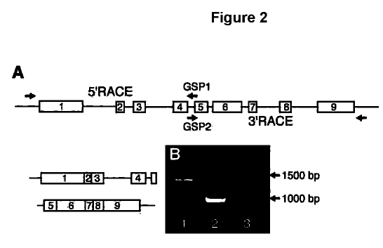

We identified two EST sequences corresponding to splice variants of human peripherin retaining intron 4 or intron 3 (BE786797 and B1832203, respectively) using the UCSC Genome Browser. Primers for RT-PCR were designed according to the retained sequence of intron 4 since we had found that peripherin splicing events involving retention of intron 4 occurred in mutant SOD1 transgenic mice (Robertson et al., 2003). Premium Total RNA extracted from human dorsal root ganglia (Clontech, USA) was chosen as the source for RT-PCR to identify peripherin splice variants since peripherin is highly expressed in this tissue (Wong et al., 1990). As our aim was to identify splicing events involving intron retention, a number of steps were taken to avoid possible confounding results due to genomic contamination. Firstly all of the RNA samples were treated with DNase prior to RT-PCR. Secondly, control experiments were performed in which reverse transcriptase was omitted showing that there was no PCR amplification of genomic DNA. Thirdly, the first strand synthesis was performed using oligo-(dT)20 to amplify mRNA only. Fourthly, parallel RT-PCR experiments were performed using off-target primers located in exon 1 and intron 3 (FIG. 1B). The product generated from these reactions had introns 1 and 2 spliced out (FIG. 1D, indicated by arrow), confirming that our samples were not contaminated with genomic DNA. Using a forward primer located in exon 3 of peripherin and a reverse primer in intron 4 (FIG. 1A), one major product of 701 bp was generated by RT-PCR, which was shown by sequencing to correspond to a transcript retaining introns 3 and 4 (FIG. 1C). Two minor products were also detected, one of 339 bp corresponded to a transcript retaining intron 4 only, the other (indicated by an asterisk in FIG. 1C) could not be identified. These transcripts were in low abundance as only the constitutively spliced peripherin isoform (in which all introns are spliced out) was detected when RT-PCR was performed using primers located in the 5′ and 3′ UTRs (not shown). Therefore 5′ and 3′ RACE using gene-specific primers in intron 4 (FIG. 2) was used to obtain the full-length transcript retaining introns 3 and 4, which was called Per 3,4.

### Expression of Per 3,4 in Transfected Cells

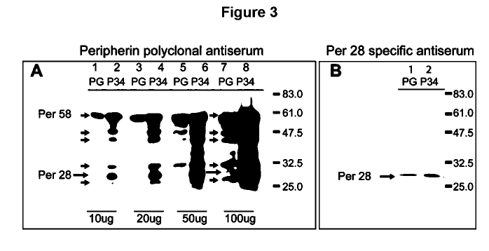

Expression of the Per 3,4 cDNA was compared with the normal full-length human peripherin gene in transfected SW13 vim (−) cells, a human cell line lacking an endogenous intermediate filament network (Sarria et al., 1994). The constitutive expression product from the peripherin gene, in which all intronic sequences are spliced out, has a molecular weight of ˜58 kDa on SDS-PAGE. For Per 3,4, the retention of intron 3 introduces a premature stop codon 30 bp downstream from the start of the intron, predicting a peripherin species of 28 kDa with a unique 10 amino acid sequence at the C-terminus. Immunoblots of cell lysates from transfected cells probed with commercially available peripherin antibody (AB1530; Chemicon) showed the expected expression of Per 58 from the peripherin gene (FIG. 3A, Lane 1). In lysates from cells expressing Per 3,4 cDNA, a species of ˜28 kDa (Per 28) was apparent, which corresponded to the molecular weight predicted from the cDNA sequence (FIG. 3A, indicated by large arrow in Lane 2). The identity of this species was confirmed using a specific antibody raised to the unique 10 amino acid sequence derived from translation of the initial 30 bp of intron 3 (FIG. 3B). There was also expression of Per 58 from the Per 3,4 cDNA, as well as expression of four other minor species of ˜48 kDa, ˜45 kDa, ˜32 kDa and ˜25 kDa (FIG. 3A, indicated by small arrows in Lane 2). Per 58 expressed from the Per 3,4 cDNA is derived from the splicing out of introns 3 and 4, indicating that Per 3,4 is itself alternatively spliced in SW13 vim(−) cells. The processing events that generated the four additional peripherin species are unknown, but unlikely to involve post-translational modifications since they are absent in similar loadings of lysates from cells expressing the peripherin gene (FIG. 3A, compare Lanes 1 and 2).

As protein loadings were increased, however, all of the species expressed from Per 3,4 cDNA also became apparent in lysates from cells expressing the peripherin gene (FIG. 3A, indicated by arrows in Lane 7). This included Per 28, verified using the specific antisera (FIG. 3B). This indicated that the additional peripherin species are in fact normal expression products from the peripherin gene, but are expressed at lower levels and at different relative stoichiometric ratios than from the Per 3,4 cDNA.

### Per 3,4 Induces Peripherin Aggregation

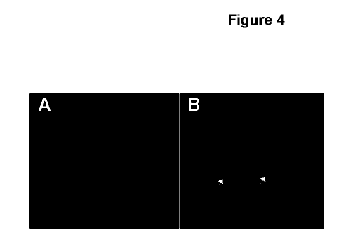

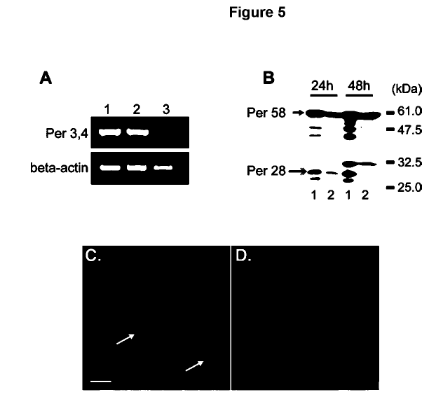

In SW13 vim(−)cells expressing the peripherin gene, peripherin displayed the normal filamentous networks that we have described previously for the mouse gene (FIG. 4A) (Beaulieu et al., 1999). In contrast, expression of Per 3,4 led to the generation of large aggregates that were immunoreactive with peripherin antibody (FIG. 4B). As the same peripherin species are expressed from the peripherin gene as from the Per 3,4 cDNA, this indicated that a change in the relative ratio's of peripherin isoform expression could induce peripherin to aggregate. To test this, we designed an interfering RNA targeting intron 3, which when coexpressed with Per 3,4 would lead to a downregulation of Per 28. Of two siRNAs designed, siRNA2 but not siRNA1 downregulated expression of transcripts retaining intron 3 (FIG. 5A, compare Lanes 2 and 3) and correspondingly reduced expression of Per 28, while maintaining expression of Per 58 (FIG. 5B, compare Lanes 1 and 2, Per 28 indicated with large arrow). This change in the relative expression levels of peripherin isoforms caused Per 3,4 to form filaments instead of aggregates (compare FIG. 5C and FIG. 5D), indicating that alterations in the relative expression levels of peripherin isoforms determines whether peripherin forms filaments or aggregates.

### Per 3,4 Expression is Upregulated in ALS

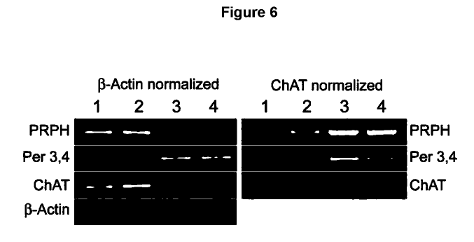

Since deregulated expression of peripherin splice variants induces peripherin to aggregate and form structures resembling peripherin immunoreactive inclusion bodies in ALS, we investigated if there was an upregulation of Per 3,4 expression in ALS spinal cord tissue compared to controls using semi-quantitative RT-PCR. As the cellular profile is different in disease spinal cord tissue compared to normal tissue (loss of motor neurons and proliferation of non-neuronal inflammatory cells i.e. microglia and astrocytes in disease tissue), two sets of primers were used for normalization; β-actin to normalize for total mRNA, and choline acetyltransferase (ChAT) to normalize for total neuronal mRNA. Findings showed a dramatic upregulation of the Per 3,4 transcript in ALS spinal cord compared to controls whether β-actin or ChAT was used for normalization (FIG. 6, compare lanes 1 and 2 with 3 and 4). The normal peripherin transcript (PRPH) was also upregulated when samples were normalized to ChAT, consistent with our earlier findings showing an upregulation of peripherin expression in ALS (Robertson et al., 2003).

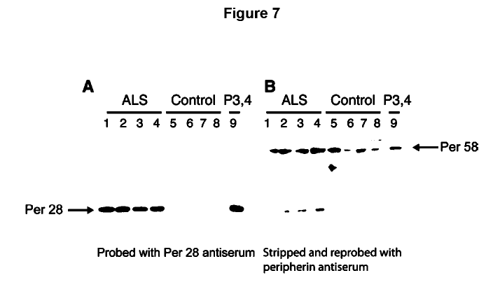

We next tested whether the increase in expression of the Per 3,4 transcript in ALS tissue led to a corresponding increased expression of Per 28, the major expression product for the Per 3,4 cDNA. Equivalent amounts of TX-100 preparations (which enriches for intermediate filament proteins) from lumbar spinal cord of four ALS cases and four control cases were probed by immunoblotting using the Per 28 specific antisera (FIG. 7A). The control samples were comprised of one non-neurological disease case (Lane 8) and three with neurological diseases other than ALS (two multisystem atrophy, Lanes 5 and 6, and one corticobasal degeneration, Lane 7). The results in FIG. 7A show that there is a pronounced increase in expression of Per 28 in all four of the ALS cases with no detectable expression in any of the control cases. Reprobing of the blots with commercially available peripherin antibody (AB1530) showed the slight increase in Per 58 expression in the ALS cases relative to controls (FIG. 7B). The increase in Per 28 expression, however, was relatively higher than this increase in Per 58 indicating that the appearance of Per 28 is not due to an overall increase in peripherin expression.

Association of Per 28 with Peripherin Pathology in ALS

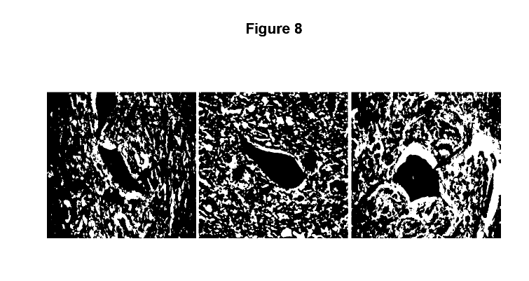

We have shown that there is an upregulation of the Per 3,4 splicing intermediate in ALS spinal cord compared to controls, and a corresponding increased expression of Per 28, the major expression product from the Per 3,4 transcript. Since we have shown that upregulated expression of Per 3,4 induces peripherin aggregate formation, we investigated whether Per 28 was associated with peripherin pathology in ALS. Immunocytochemical labeling of ALS spinal cord tissue with the Per 28 antibody showed distinct labeling of peripherin aggregates resembling round inclusions in the cytoplasm of motor neurons (FIG. 8). These findings indicated that not only was there increased expression of Per 28 in ALS but that it was also associated with disease pathology.

### Development of a Sandwich ELISA Assay for Detection of Per 3,4

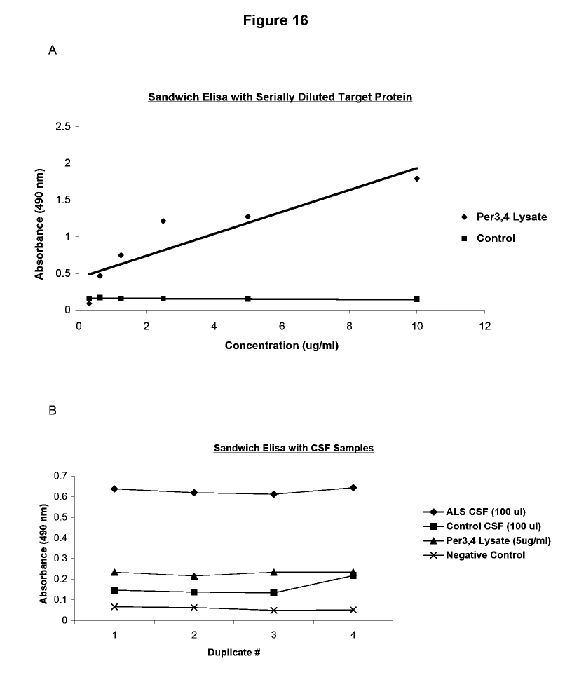

The Per 3,4 rabbit antisera was used for capture and commercially available chicken peripherin antisera for detection, using soluble lysates from Per 3,4 expressing cells as substrate. Using this approach an ELISA assay was developed to detect Per 3,4 in biological samples and validated using CSF from ALS subjects (FIG. 16). FIG. 16B shows detection of Per 3,4 in CSF from ALS and a control sample using the Sandwich ELISA. A CSF sample from a 60-year-old male with sporadic ALS and a control sample from a 62-year-old male with progressive dysarthria were tested. Note robust detection of Per 3,4 in CSF from the ALS patient (FIG. 16). CSF data using the antibody to Per 3,4 is shown in Table 11.

The basis of the ELISA assay was as follows: capture antibody: affinity purified Per 3,4 rabbit polyclonal antibody; sample: soluble lysates from cells expressing Per 3,4; CSF; Blood plasma; and the detection antibody: chicken polyclonal peripherin antibody.

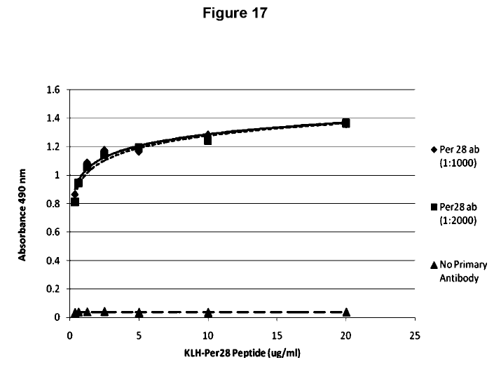

KLH-tagged Per 3,4 peptide has been validated for use to provide a quantitative ELISA (FIG. 17). A quantitative sandwich ELISA will be developed using affinity purified Per 3,4 rabbit polyclonal antibody for capture and Per 3,4 mouse monoclonal (in production) for detection.

Material and methods for Identification of Peripherin Splice Variants

### RNA and Semi-Quantitative RT-PCR

All human RNA samples for RT-PCR were obtained commercially either from Clontech or Ambion Inc., and treated with DNase prior to RT-PCR to remove any genomic DNA contamination of the RNA sample. Control RT-PCR experiments with omission of the reverse transcriptase were performed in parallel with all experiments to further ensure results obtained were not due to genomic contamination. Premium Total RNA from human dorsal root ganglia was from Clontech (Catalogue number: CR2496). Control total RNA samples from human lumbar spinal cord was pooled from 49 male/female Caucasians ages from 15 to 66 (Catalogue number: 64113-1, Clontech); and from a normal 65 years old male Caucasian (Catalogue number: B6840, Ambion Inc.). Total RNA from ALS human lumbar spinal cord (Catalogue: B6162, Ambion Inc.) was from two 70 years old male Caucasians. RNA samples were pre-treated with DNase prior to experiments. The cDNAs were synthesized from 1 pg of total RNA with Oligo(dT)₂₀ using the SuperScript III First-Strand Synthesis System for RT-PCR from Invitrogen Life Technologies following the manufacturer's protocol. The ratio of mRNA transcripts was estimated using semi-quantitative RT-PCR normalized using primers specific for β-actin (Ambion Inc.) and choline acetyltransferase (ChAT). For amplifying the normal peripherin gene transcript, PRPH, primers were located in exon 3 and exon 6 (see Table 9). For PCR, 1 μl of template cDNA solution was placed in 25 μl of reaction solution containing 1× PCR Buffer [20 mM Tris-HCl (pH8.4), 50 mM KCl], 200 μM dNTP, 2 mM MgCl₂, 5% DMSO, 2 μM of each primer and 2U Platinum Taq DNA Polymerase (Invitrogen Life Technologies). The amplification conditions for the peripherin splice variant consisted of initial denaturation at 95° C. for 5 min, followed by 40 cycles of 95° C. for 30 s, 62° C. for 30 s and 72° C. for 30 s in a GeneAmp PCR system 9700 (Perkin-Elmer Applied Biosystems). Cycle numbers for the RT-PCR amplifications of p-actin, ChAT and PRPH was 25 cycles, 35 cycles and 30 cycles, respectively.

Cloning of Full Length Per 3,4 cDNA using 5′ and 3′ RACE

According to the sequence of intron 4 of peripherin obtained from Genbank (Accession number:NM_{—}006262), we designed 5′ and 3′ Gene-Specific Primers (GSP): an antisense primer (GSP1) for 5′-RACE PCR (5′-GAC AGG TCC GCG TAC TGA GM GTG G-3′) (SEQ ID NO: 30) and a sense primer (GSP2) for 3′-RACE PCR (5′-GTC CM GGT GCA AGA GCC GGG AGG-3′) (SEQ ID NO: 31). 5′-RACE-Ready cDNA was synthesized by combining 1 μg total RNA with BD SMART II™ A Oligonucleotide and 5′-RACE CDS Primer; 3′-RACE-Ready cDNA was synthesized by combining 1 μg total RNA with 3′-CDS primer. CDS primers were supplied by the manufacturer. The detailed procedure is exactly as described in the BD SMART™ RACE cDNA Amplification Kit User Manual (BD Biosciences, Catalogue number: 634914). 50 μl of RACE PCR reaction solution consisted of 2.5 μl 5′-RACE-Ready cDNA or 3′-RACE-Ready cDNA, 34.5 μl of PCR-Grade water, 5 μl of 10× Advantage 2 PCR Buffer, 1 μl of 10 mM dNTP, 1 μl of 50× BD Advantage 2 Polymerase Mix, 5 μl of 10× Universal Primer Mix, 1 μl of 10 μM GSP1 or GSP2. Thermal cycling was followed using the touchdown PCR program: 5 cycles at 94° C. for 30 s, 72° C. for 3 min; 5 cycles at 94° C. for 30 s, 70° C. for 30 s, 72° C. for 3 min; 30 cycles at 94° C. for 30 s, 68° C. for 30 s, 72° C. for 3 min. The 5′-RACE and 3′-RACE PCR products were analyzed by electrophoresis on 1.2% (w/v) agarose/ethidium bromide gels and purified using the MinElute Gel Extraction Kit (Catalogue number: 28604, QIAGEN). The isolated fragments were cloned directly into a TOPO TA Cloning vector: pCR2.1-TOPO (Catalogue number: K4510-20, Invitrogen Life Technologies). The inserts were sequenced with M13 Forward and Reverse Primers on an ABI PRISM 3100 Genetic Analyzer (Perkin-Elmer Applied Biosystems). The full-length cDNAs were generated by blunt-end ligation of the 5′-fragment and 3′-fragment, then subcloned into the BamH1/EcoR1 sites of pcDNA3.1 (Catalogue: V795-20, Invitrogen Life Technologies) and subjected to sequence analysis for final verification.

### Cloning of Full Length Human Peripherin Gene (PRPH)

A probe specific to the human peripherin gene was used to identify BAC clone number 977B10 from the RPCI-11 Human BAC Clones available from the Canadian Institutes of Health Research (CIHR) Genome Resource Facility. The BAC DNA was extracted from a bacterial culture inoculate using alkaline lysis and the full-length human peripherin gene was obtained from 977B10 BAC DNA by simultaneous digestion with EcoRI and EcoRV. A band corresponding to approximately 5000 bp was purified using the MinElute Gel Extraction Kit (Qiagen), subcloned into the EcorR1/EcoRV site of pcDNA3.1(−), and subjected to sequence analysis for verification.

### Transient Transfection

A human adrenal carcinoma cell line, SW13vim(−), was transfected using Lipofectamine 2000 (Invitrogen Life Technologies) following manufacturer instructions. Ectopic expression of peripherin was detected using peripherin polyclonal antibody (AB1530; Chemicon International, Inc.

Preparation of siRNA Targeting Per 3,4

The selected siRNA sequences for Per 3,4 was from nucleotides 129-137 for RNAi-1 and 262-280 for RNAi-2, relative to the start of intron 3. The sequences were designed using the program available at http://www.dharmacon.com and a BLAST search revealed no substantial homology of the chosen sequences to other genes. The oligonucleotides were synthesized by Integrated DNA Technologies and diluted to 1 μg/μl. The forward and reverse primers were annealed and cloned into linearized pSupressorNeo vector according to the manufacturer's protocol (IMGENEX Corporation).

### Immunocytochemistry of Cultured Cells

SW13vim(−) cells grown on glass coverslips were fixed in methanol for 5 min at −20° C. and rehydrated in PBS. Immunocytochemistry was performed using an antibody recognizing peripherin (AB1530) and used at 1:1,000 diluted in PBS. Antibody distribution was visualized by epifluorescence microscopy after incubation with secondary antibodies, Alexa Fluor 594 or 488 diluted 1:350 in PBS (Molecular Probes).

### Immunoblotting

Cells were harvested in 62.5 mM Tris, pH 6.8, containing 2% SDS and 10% glycerol, and assayed for total protein using the bichinconinic acid assay (Sigma-Aldrich). Loadings of 10-100 μg of protein were analyzed on 10% (wt/vol) SDS-polyacrylamide gels and then blotted to polyvinyldiflouride (PVDF) membrane. For immunoblotting, membranes were incubated with antibodies recognizing peripherin (MAB1527 or AB1530) diluted 1:5,000 in blocking solution (3% skimmed milk powder in TBS-Tween). Antibody binding was revealed with the ECL detection system (NEN Life Science Products).

### Generation of Splice Variant Specific Antibody

A rabbit polyclonal antiserum was raised to a synthetic peptide corresponding to the sequence derived from translation of the first 30 bp of intron 3 (VSGPGIRGGF) and specificity verified by immunoblotting.

### Human Spinal Cord Tissue

TX-100 extractions were prepared from ˜100 mg lumbar spinal cord tissue taken from four sporadic ALS cases and four controls. The control cases comprised one with no indication of neurological disease and three with neurological disease (two multisystems atrophy and one corticobasal degeneration). Briefly, samples were homogenized in 1 mL high salt buffer (HSB; 50 mM Tris, pH7.5, 750 mM NaCl, 5 mM EDTA) containing protease inhibitor cocktail (Sigma-Aldrich) and centrifuged at high speed in Eppendorf microcentrifuge at 4° C. for 10 mins. The resulting pellet was rehomogenized in HSB containing 1% TX-100 and centrifuged as before. This extraction was repeated twice. The final pellet was then homogenized in HSB containing 1M sucrose and centrifuged as before to remove myelin. Equal protein amounts from the different samples were resolved on 10% SDS-PAGE and probed by immunoblotting with Per 28 specific antisera or polyclonal peripherin antibody (AB1530). For immunocytochemistry, paraffin embedded sections (6 μm) of lumbar spinal cord were rehydrated through a series of washes in graded ethanol and finally in water. Sections were pretreated with 10 mM sodium citrate, pH 6.0 for epitope retrieval and incubated with Per 28 splice variant specific antisera diluted 1:1000 in Dakocytomation Antibody Diluent overnight at 4° C. Antibody labeling was revealed using DakoCytomation Envison™ System according to the manufacturer's instructions using 3,3′-diaminobenzidine as chromagen. Labeled sections were visualized using a Leica DM 6000 microscope and digital images obtained with a Micropublisher 3.3 RTV color camera and Openlab imaging software (Improvision).

### Example 2

### Results

### Identification of Two Alternatively Spliced Variants of NFL

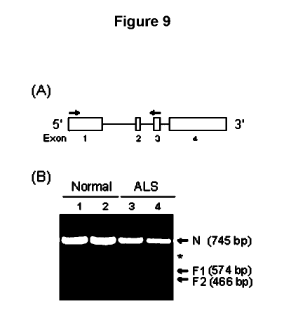

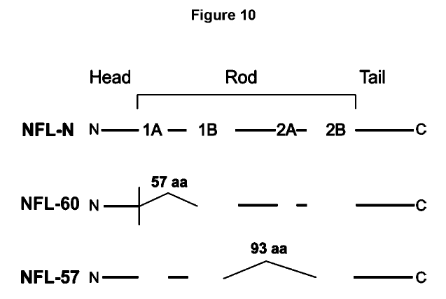

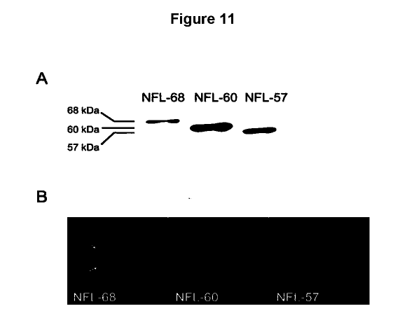

Because differential expression patterns of peripherin splice isoforms has been found in ALS patients, we examined whether NFL, which, like peripherin, is another major constituent of intraneuronal inclusions in motor neurons of ALS cases, has alternative splice variants whose expressions may similarly be altered in ALS. Although several human NFL mRNA sequences with partial loss of exon 1 have been identified and documented in GenBank, none has been confirmed to be expressed and translated into functional proteins in human tissues. RT-PCR amplifying exons 1 and 2 of human NFL has revealed the in vivo expression of such spliced variants of human NFL in spinal cord tissues of both normal individuals and ALS patients (FIG. 9). The NFL variant 60 is exclusive to one ALS patient while the expression of NFL-57, is positive in all four samples, although variably expressed. Interestingly there is a slight diminishment in expression of the normal NFL species (NFL-68) in the ALS samples compared to controls (compare Lanes 1 and 2 with 3 and 4). Together, these results illustrate a shift in ratio of NFL and its variants between normal and diseased cases. NFL-57 lacks 99 amino acids that constitute the majority of the rod domain in NFL (FIG. 10). Alternative splicing of NFL-60 results in deletion of 57 amino acids from the entire helix 1a portion of the rod domain (FIG. 10). As the rod domain drives NF assembly through formation of a coiled-coil dimer with other NF subunits, its deletion or disruption as observed in NFL-57 and 60 is likely to abolish the assembly capability of the proteins. Subsequent expression of cDNAs encoding the different NFL isoforms generated proteins of the expected molecular weights in cell extracts analyzed by SDS-PAGE: 68 kDa for NFL, 57 kDa for NFL-57 and 60 kDa for NFL-60 (FIG. 11A). ExPASy Compute pl/mw tool predicted molecular masses of 64 kDa, 53 kDa and 57 kDa respectively for NFL, 57 and 60. Such slight discrepancies between theoretical and experimental molecular masses are expected and acceptable.

Assembly Properties of NFL Isoforms with Neurofilament Subunits

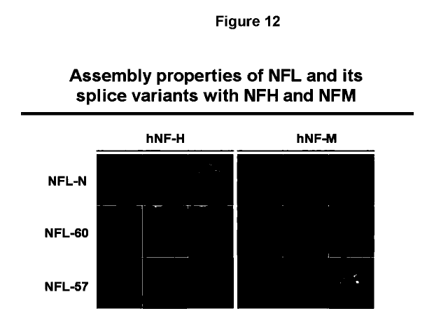

As intraneuronal NF accumulation is a prominent feature of ALS, we investigated whether NFL-57 and NFL-60 are capable of co-assembly into a filamentous network with NFM and NFH. The assembly characteristics were explored by co-transfecting NFL isoforms with NFH or NFM into SW13 vim(−) cells which are devoid of endogenous cytoplasmic intermediate filament network (Sarria et al., 1990). Immunocytochemical analysis first showed that neither NFL nor the splice variants were capable of self-polymerization (FIG. 11B). Next, it was demonstrated that NFL co-assembled and networked with NFM and NFH whereas both NFL splice variants were found to be assembly incompetent (FIG. 12). In NFL-60/NFH and NFL-57/NFH transfected cells, the distribution of the proteins gave a speckled appearance with punctate aggregates scattered throughout the cytoplasm (FIG. 12). In addition to this speckled phenotype, expression of NFL-60 and NFL-57 with NFM induced formation of large aggregates (FIG. 12). These results collectively demonstrated that filament assembly was disrupted in the presence of NFL-57 and NFL-60, indicating the potential role of the splice variants in establishing intraneuronal inclusions characteristic of ALS.

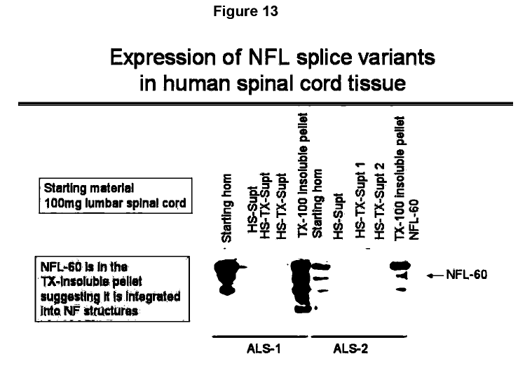

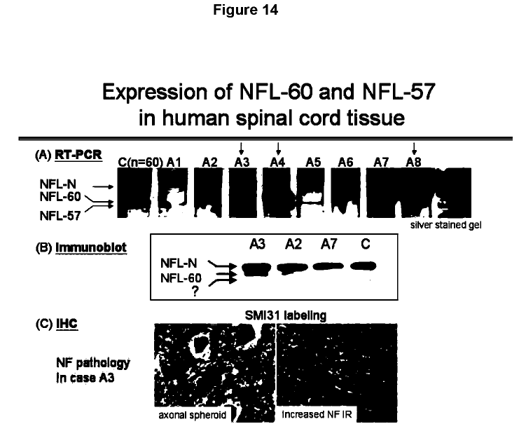

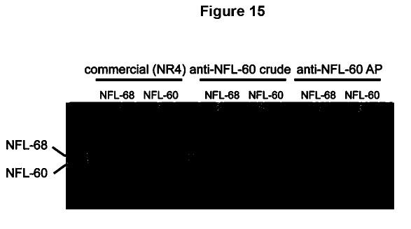

Lumbar spinal cord samples from two ALS cases (ALS-1 and ALS-2; FIG. 13), were processed through a series of extraction buffers all containing protease inhibitors; high salt (HS) buffer (50 mM Tris, pH7.5, 750 mM NaCl, 5 mM EDTA); HS buffer including 1% Triton X-100 (HS-TX); HS buffer including 1M sucrose (HS-S). It was found that NFL-60 partitioned to the Triton X-100 insoluble fraction, indicating that NFL-60 is integrated within higher polymeric structures (FIG. 13). Expression of NFL-60 and NFL-57 was analyzed in another additional eight ALS cases. The mRNA for NFL-60 was found in three of eight ALS samples, whereas NFL-57 was detected in control and four of the eight ALS samples (FIG. 14A). NFL-60 is clearly absent from the control sample (consisting of a mixture of RNA from a total of non-related individuals) and is likely that the variation in presence of absence of NFL-57 (which is present in control) reflects the quality of the RNA from the ALS cases which had a range of postmortem delays (FIG. 14A). Lumbar spinal cord samples from cases A3, A2, A7 and a control sample were analyzed by immunoblotting (FIG. 14B). NFL-60 was detected at the protein level in two of the ALS cases (A2 and A3), but not in the control case. Immunohistochemical analysis of case A3 showed the presence of neurofilament pathology that correlated with the presence of NFL-60. A rabbit polyclonal antiserum to NFL-60 was generated using the unique peptide sequence NDLKSIRDLR (SEQ ID NO: 8). The crude NFL-60 antiserum showed remarkable specificity for NFL-60 on immunoblots compared to the commercially available antibody, NR4 (FIG. 15, compare Lanes 1 and 2 with 3 and 4). The slight cross-reactivity of the NFL-60 antiserum with the normal NFL variant NFL-68 (indicated by arrow in Lane 3 of FIG. 15) was completely removed by affinity purification using a GST-NFL-68 fusion protein (FIG. 15, compare Lanes 3 and 5).

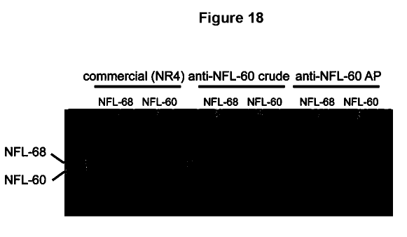

Rabbit polyclonal antisera has been raised to synthetic peptide corresponding to the unique sequence in NFL-60 (FIG. 18) providing NFL-60 specific antisera. Approximately 10 μg loadings of total lysates from cells expressing NFL-68 (lanes 1, 3, 5) or NFL-60 (lanes 2, 4, 6) were resolved on 10% SDS-PAGE and transferred to PVDF membrane. The membrane was divided into three pieces and probed with the antibodies as shown: Commercially available monoclonal to NFL, NR4; Crude NFL-60 specific antisera; Affinity purified NFL-60 antibody. NR4 antibody labels both NFL-68 and NFL-60 (Lanes 1 and 2) whereas the crude NFL-60 antiserum shows increased specificity for NFL-60 (compare lanes 1 and 2 with 3 and 4). The slight cross reactivity of the NFL-60 antisera with NFL-68 (arrow in lane 3) is removed by affinity purification (note lack of signal in Lane 5).

### NFL Discussion

Despite the effort of many studies focusing on the different aspects of the neurodegenerative pathology of ALS, many questions remain unresolved. Here, we have shown for the first time expression of two NFL alternatively spliced variants, NFL-60 and NFL-57, at both the mRNA and protein levels in SW13 vim(−) transfected cells and more importantly in human spinal cord tissue. The former existed exclusively in ALS cases while expression of the latter was found both in control and ALS samples. Interestingly, the normal NFL transcript (NFL-68) appeared to have slightly decreased expression in ALS cases relative to controls. In contrast to NFL-68, the NFL splice variants, NFL60 and NFL-57, were incapable of network assembly with NF subunits. These findings represent the first evidence that disturbed metabolism and expression pattern of NFL and its alternatively spliced variants may potentially contribute to neurodegeneration by inducing formation and accumulation of NF aggregates in motor neurons.

Sequence analysis shows that alternative splicing within exon 1 of the two NFL isoforms of interest results in partial loss of the rod domains of NFL-60 and 57. The NFL rod domain mediates dimerization of NF proteins in proper establishment of a filamentous network (Liem, 1993). The substantial sequence conservation of the NFL rod domain among a diversity of species, from Xenopus to human (Fuchs and Weber, 1994), indicates a tight preservation in functionality and a critical role NF assembly has in the proper survival of organisms among different phyla. In Charcot-Marie-Tooth disease, single mutations in the NFL rod domain have been found to completely eliminate the ability of NFL coiled-coil interactions with other NF subunits thereby causing severe neurodegeneration of motoneurons (Perez-Olle et al., 2002). The present co-transfection studies have shown that NFL-60 and 57 with defective rod domains are similarly defective in co-assembly with other NF subunits. A complete lack of filamentous network, with punctate proteins dominating the cytoplasmic space, was observed when NFL-57 and NFL-60 were co-transfected with either NFH or NFM. Occasionally, small aggregated structures appeared in random cells co-expressing NFM and the NFL isoforms. One should keep in mind that the transfection data represented only the assembly characteristics of NFL variants with NF subunits but was not reflective of the pathological cellular phenotype of ALS. The intraneuronal inclusion characteristic of ALS arises as a result of complicated interactions among a variety of cellular components. Nevertheless, the present inventors have demonstrated that not only are the NFL splice variants incapable of establishing a functional filamentous network with NF subunits due to altered rod domains, they induce the formation of aggregates and may therefore participate in causing neuronal inclusion and subsequent motor neurodegeneration in ALS. Single transfection of NFL resulted in punctate staining similar to that observed in spliced variant transfected cells. Perhaps the assembly property of NFL has stringent requirements in which its level of expression above or below a certain range can render the protein incapable of self-assembly.

Differential expression pattern of NFL and its splice variants in ALS was observed, with unique expression of NFL-60 found in ALS cases but not in controls. NFL-57 exhibited variable expression whereas there was a downregulation of normal NFL (NFL-68) in ALS cases relative to controls. The decline of NFL-68 expression in ALS cases may be explained in that a defined amount of NFL total mRNA may be used to generate constitutively and alternatively spliced variants. Regardless of the presence or absence of NFL-60, the variation in NFL-68 and NFL-57 expression represents a shift in the respective proportion of NFL with its splice variants as well as with other NF subunits. As a fine balance among NF proteins and isoforms may be essential in normal filamentous networking, such perturbation of the intra- and inter-ratio of NFL with its splice variants and NF subunits may promote formation of NF aggregates and in consequence cause motor neuron death.

Monoclonal antibodies are generated that are specific for the NFL splice variants by identifying unique sequences around the joined sites of spliced exons. Moreover, transfection studies with NFL variants are performed with bicistronic constructs to minimize the limitations presented by variation in transfection efficiency among different clones.

### Example 3

### Further Splice Variants

It is expected that components involved in tight regulation of alternative splicing mechanisms play a role in ALS pathogenesis. Aberrant splicing arises from mutations such as single nucleotide polymorphism (SNP) in the splice site, exonic or intronic splicing enhancers (ESE, ISE) or silencers (ESS, ISS). In addition to these cis-acting elements, trans-acting serine/arginine-rich splicing factors (SR proteins) similarly harbor mutations that may influence selection of alternative splice sites. Furthermore, defects in upstream molecules that mediate phosphorylation of SR proteins in turn lead to pre-mRNA splicing dysregulation. Indeed, mutations in splicing regulatory elements leading to disturbance of mRNA splicing and isoform ratios have been identified in the disease pathology of sporadic tauopathies (Hartmann et al. 2001; Hernandez et al. 2004; Stoilov et al. 2004). Therefore, it is likely that ALS follows similar disease mechanisms. Studies to screen for presence of SNPs in cis-acting sequences are beneficial to elucidate the role of splicing regulatory elements in ALS pathogeneisis. In summary, ALS is a heterogeneous disease involving multiple genetic and possibly environmental components acting synergistically to bring upon the pathological phenotypes observed. The discovery of two alternatively spliced variants of NFL whose relative expressions are altered in ALS patients parallels with previous studies on peripherin isoforms and provides additional support for the potential role(s) of disturbed IF metabolism in the formation of neuronal aggregates and ultimately the disease progression in ALS.

### Materials and Methods

### Identification of Alternatively Spliced Variants of NFL

Isolated RNA from ALS cases and normal individuals was subjected to reverse transcription polymerase chain reaction (RT-PCR) with forward primer 5′-CTG GAT CGT TGA TGC CCA G-3′ (182-200 bp; 19 bp) (SEQ ID NO:32) and reverse primer 5′-CGG CTC TCG GAC ACC TCG TC-3′ (907-926 bp; 20 bp) (SEQ ID NO:33).

Normal and ALS human lumbar spinal cord total RNA were purchased from BD Biosciences and Ambion Inc. respectively. RNA samples were subjected to reverse transcription polymerase chain reaction (RT-PCR) to synthesize cDNA from 1 μg of total RNA with Oligo(dT)₂₀ using SuperScript III First-Strand Synthesis System for RT-PCR according to manufacturer's protocol (Invitrogen). For PCR, 1 μl of template cDNA was added to 19 μl of reaction solution containing: 10 μl of 2× GeneAmp Fast PCR Master Mix with AmpliTaq Polymerase; GeneAmp PCR buffer; dNTPs; MgCl₂ and stabilizers (Applied Biosystems), 1 μl of 10 μM forward primer, 1 μl of 10 μM reverse primer and 7 μl of H₂O. Linear amplification of exons 1 and 2 was achieved in a 9800 Fast Thermal Cycler (Applied Biosystems) by an initial denaturation at 95° C. for 10 sec, followed by 38 cycles of denaturation at 95° C. for 0 sec and annealing at 66° C. for 20 sec. Reaction was terminated by a 10 sec extension step at 72° C. RT-PCR was normalized using primers specific for β-actin (Ambion Inc.). PCR products of 745 bp (NFL-68), 574 bp (NFL-60) and 466 bp (NFL-57) were separated on 1.5% agarose gels; 2 splice variants (574 bp and 466 bp) were gel purified according to QIAquick Gel Extraction Kit (QIAgen) and subcloned into pCR 2.1-TOPO plasmid vector by TOPO TA Cloning Kit according to manufacturer's instruction (Invitrogen) and later sequenced by AGCT Corporation (Toronto, Canada) using forward primer 5′-GTAAAACGACGGCCAG-3′ (SEQ ID NO:34) and reverse primer 5′-CAGGAAACAGCTATGAC-3′ (SEQ ID NO:35) from TOPO vector.

For cloning of human full-length NFL-60 and NFL-57, 1 μg of human lumbar spinal cord total RNA was reversely transcribed with an adapter primer (GGC CAC GCG TCG ACT AGT AC(T)17) (SEQ ID NO:36) to obtain the cDNA, followed by two step of nested PCR. For the first round of PCR, 1 μl of cDNA was placed in 24 μl of PCR reaction solution containing 1× PfuUltra buffer, 200 μl dNTPs, 2 μl of Human NFL 5′-UTR forward primer (5′-GCACACAGCCATCCATCCTC-3′) (SEQ ID NO: 40: −97 to −77 bp from first ATG of SEQ ID NO: 6) and 2 μl of Reverse primer of adapter sequence (5′-GGC CAC GCG TCG ACT AGT AC-3′) (SEQ ID NO:37) and 1.25 U of PfuUltra DNA polymerase (Catalogue number: 600380-51, Stratagene). Amplification was achieved with an initial denaturation at 95° C. for 45 sec, followed by 25 cycles of 95° C. for 45 sec, 60° C. for 45 sec and 72° C. for 2 min in GeneAmp PCR System 9700 (Applied Biosystems). The PCR product was diluted at 1/100, and was subjected to a second round of PCR amplification that utilized forward primer with Not I restriction enzyme site (5′ATAAGAATGCGGCCGCGCACACAGCCATCCATCCTC-3′ (SEQ ID NO:38); underlined sequence corresponds to −97 to −77 bp of SEQ ID NO:6) and reverse primer from 3′-UTR of NFL gene with Bam HI restriction enzyme site (5′-CGCGGATCCGCGTTCGGTATAACTTTATTTACT-3′ (SEQ ID NO:39); (underlined sequence corresponds to 407-430 bp from TGA stop codon in SEQ ID NO: 6). The PCR conditions were identical to the formal amplification process. The second PCR products were analyzed by electrophoresis on 1.2% (w/v) agarose/ethidium bromide gels and purified using the MinElute Gel Extraction Kit (Catalogue number: 28604, QIAGEN), then subcloned into the Not I/Bam HI sites of pcDNA3.1(−) (Catalogue number: V795-20, Invitrogen Life Technologies). All the constructs were subjected to sequence analysis for final verification.

### Transfection

SW13 vim(−) cells lack an endogenous cytoplasmic intermediate filament network and are routinely used for studies of neurofilament protein assembly (Sarria et al., 1990) SW13 vim(−) cells were grown in Dulbecco's modified eagle medium (DMEM) with 5% fetal bovine serum (FBS) and 1% penicillin/streptomycin. Cells were plated in 4 well tissue culture dishes (Nunclon) containing coverslips and transfected at 90-95% confluency using Lipofectamine 2000 (Invitrogen) and 0.8 μg of each plasmid DNA. The transfections were NFL; NFL-60; NFL-57 either individually or together with NFH or NFM. The plasmid DNAs were diluted into 50 μl of DMEM without FBS and 2 μl of Lipofectamine 2000 was diluted in another 50 μl of DMEM without FBS for each transfection. Diluted DNAs and Lipofectamine reagent were combined and incubated at room temperature for 30 minutes. The mixture was added to the plated cells along with 100 μl of DMEM incomplete (without penicillin or FBS). Transfection was allowed to occur for 5 hours before addition of 500 μl of complete DMEM. Cells were incubated for 48 hours to allow DNA expression.

### Protein Extraction and Immunoblotting

Transfected SW13 vim(−) cells were washed with PBS after culture medium was discarded, and resuspended in PBS containing protease inhibitor-cocktail (Sigma) by scraping the bottom of the well. The dissociating solution was collected and centrifuged for 1.5 minutes at 13200 rpm at 4° C. The supernatant was removed by aspiration and the pellet resuspended in 60 μl pf PBS with protease inhibitor cocktail before the addition of 30 μl of boiled 6% (w/v) SDS to give a final SDS concentration of 2% (w/v). The protein concentrations were determined by Bichinconinic Acid Assay (Signma). 10 μg of proteins were loaded onto 10% SDS-PAGE gel and then blotted to polyvinyldiflouride (PVDF) membrane. The membrane was incubated in polyclonal anti-NFL primary and anti-rabbit secondary antibodies, both at dilutions of 1:5000.

### Immunocytochemistry

Transfected SW13 vim(−) cells were rinsed with pre-warmed PBS and fixed for 5 minutes at −20° C. with methanol. Fixed cells were washed 3 times with PBS and incubated for 30 minutes at room temperature with polyclonal anti-NFL, monoclonal anti-NFH (Smi31, Smi32) and monoclonal anti-NFM (NN18) primary antibodies at dilutions of 1:1000 in PBS. After 3 PBS washes, cells were incubated for 30 minutes in the dark at room temperature with secondary antibodies, anti-rabbit and anti-mouse IgG conjugated to Alexa Fluor 594 or Alex Fluor 488 diluted at 1:300. Fluorescent antibody distribution was visualized by epifluorescence microscopy.

### Immunohistochemistry

For immunocytochemistry, paraffin embedded sections (6 μm) of lumbar spinal cord were rehydrated through a series of washes in graded ethanol and finally in water. Sections were pretreated with 10 mM sodium citrate, pH 6.0 for epitope retrieval and incubated with neurofilament diluted 1:1000 in Dakocytomation Antibody Diluent overnight at 4° C. Antibody labeling was revealed using DakoCytomation Envison™ System according to the manufacturer's instructions using 3,3′-diaminobenzidine as chromagen. Labeled sections were visualized using a Leica DM 6000 microscope and digital images obtained with a Micropublisher 3.3 RTV color camera and Openlab imaging software (Improvision).

Biochemical Fractionation of NFL-60 from ALS Spinal Cord

Approximately 100 mg of ALS or control spinal cord tissue was homogenized at 4° C. in low salt buffer containing 50 mM Tris (pH 7.5), 150 mM NaCl, 5 mM EDTA and protease inhibitor cocktail. The homogenates were then centrifuged at 16,000 g for 10 min at 4° C. The pellet fractions were further homogenized in high salt (HS) buffer containing 1% Triton X-100 and centrifuged at 16,000 g for 10 min at 4° C. The resultant pellets were treated to a final homogenization in HS buffer containing 1 M sucrose and re-centrifuged to remove lipids. The final pellet was solubilized in 2% (w/v) SDS in PBS. Samples were assayed for protein concentration using the bicinchoninic acid assay and then diluted in 2× loading buffer [Tris-HCL, pH 6.8, 30% (w/v) glycerol, 4% (w/v) SDS, 10% (v/v) β-mercaptoethanol and 0.02% (w/v) bromophenol blue] and boiled for 5 min. Loadings of 10-15 μg were routinely analysed on 10% SDS-polyacrylamide gels and then blotted to polyvinyldifluoride (PVDF) membrane. For comparison of Triton X-100 soluble versus insoluble fractions, equal volumes of sample were used. For immunoblotting, membranes were blocked with 3% (w/v) skimmed milk powder in Tris-buffered saline containing 0.2% Tween-20 (TBS-T) for 1 hour at RT, then with polyclonal peripherin antibody diluted 1:5000 in the blocking solution overnight at 4° C. A monoclonal GAPDH antibody (H86504M, Biodesign, ME) diluted 1:5000 was used as the internal loading control. Antibody binding was revealed using HRP-conjugated anti-rabbit or anti-mouse IgG and an ECL detection system (NEN Life Science Products, Woodbridge, ON).

### Generation of NFL-60 Splice Variant Specific Antibody

A rabbit polyclonal antiserum was raised to a synthetic peptide corresponding to the sequence derived from translation of the first 30 bp of intron 3 (NDLKSIRDLR) and specificity verified by immunoblotting.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

Human peripherin gene (from UCSC Genome Browser)
Position of intron 3 from 1^{st} ATG 1328 bp-1688 bp
(361 bp)
Position of intron 4 from 1^{st} ATG 1857 bp 1947 bp
(91 bp)
Start and stop codons shown in bold, exons shown
in capital letters

(SEQ ID NO: 1)

cctcgcagcggtctgcggctccttcccagcccccggcctagctctgcgaa
cggtgactgcccatccttggccgcaATGAGCCACCACCCGTCGGGCCTCC
GGGCCGGCTTCAGCTCCACCTCATACCGCCGTACCTTCGGTCCACCGCCC
TCACTATCCCCCGGGGCCTTCTCCTACTCGTCCAGCTCCCGCTTCTCCAG
CAGCCGCCTGCTGGGCTCCGCGTCCCCGAGCTCCTCGGTGCGCCTGGGCA
GCTTCCGTAGCCCCCGAGCGGGAGCGGGCGCCCTCCTGCGCCTGCCCTCG
GAGCGCCTCGACTTCTCCATGGCCGAGGCCCTCAACCAGGAGTTCCTGGC
CACGCGCAGCAACGAGAAGCAGGAGCTGCAGGAGCTCAACGACCGCTTCG
CCAACTTCATCGAGAAGGTACGCTTTCTGGAGCAGCAGAACGCGGCCCTG
CGCGGGGAGCTGAGCCAAGCCCGGGGCCAGGAGCCGGCGCGCGCCGACCA
GCTGTGCCAGCAGGAGCTGCGCGAGCTGCGGCGAGAGCTGGAGCTGTTGG
GCCGCGAGCGTGACCGGGTGCAGGTGGAGCGCGACGGGCTGGCGGAGGAC
CTGGCGGCGCTCAAGCAGAGgtcagggggcagggctgggccgctgccgtc
gaggcgaggtcgaagcggccgtcgaggcggctgctcttgcctcccctcgc
ttcccctctccatcagcagcccaagggtgtggctccccttaccaacccag
gtgtgtgcgggcagcatccctgcccacgggctccaagtgccccccgctac
ccctttgctctgagtgtttggggaggtgggagaagtgggtatctgtgcct
cccctgagtaatgaggaaacccccttttcagctcccagtccgttagagac
aatgcggggcaattccattagacagcctcagccctccatttagagtcctg
ggcagcagaacagcctctaaccggatcctggggggcgtgcggtctggggt
gcgagctgggcggcgaccccgcagttcagcctctgcacgctcttcccgtc
agGTTGGAGGAGGAGACGCGCAAGCGGGAGGACGCGGAGCACAACCTCGT
GCTCTTCCGCAAGgtgagtccgagcccctctccgagttcagcctccccac
cgctacccccgatctcagtatccagaggtggcatcggtgggcgcggggag
aagggggtaacccagatgcctcctgaggcagacagggaaggcctggtcct
tccttggtctgcgcagcccctaacttatcttgaacctccactgccacccc
tcgaagGACGTGGACGATGCCACTCTGTCCCGCCTGGAACTAGAGCGCAA
GATTGAGTCTCTGATGGATGAGATTGAGTTCCTCAAGAAGCTGCACGAGG
AGgtaagtgggcccggtatcaggggcggtttctgaggttgtggggtggtc
tcgctggagctggcgggtggagcggaggcatcgccctggggatcaggacg
atgctgggtagacgcagcccctccaccctagtctacaggtggttagactc
ccacccttgcgccacctggcggcgggcagcggggctgtacctccgaaacc
tggcctctggtctcgcgcccgcgggggcgcagggctgtacgccctgccct
ccctggcgcccacttctgttcgttcaagcgtttcttctcttttctgtgca
cgaactgcgtggcccgcgtggaatttgcgccgctgtccatcctctgcctg
ctcccggccgtagGAGCTGCGAGACCTGCAGGTGAGTGTGGAGAGCCAGC
AGGTGCAGCAGGTGGAGGTGGAAGCCACGGTGAAGCCCGAGCTGACGGCA
GCGCTGAGGGACATCCGCGCGCAGTACGAGAGCATCGCCGCGAAGAACCT
GCAGGAGGCGGAGGAGTGGTACAAGTCCAAGgtgcaagagccgggagggc
ctgcgaggcgggacgctggggtggtgtcgcgcgtcccagccgactaaagc
ctgggttacccccacttctcagTACGCGGACCTGTCCGACGCTGCCAACC
GGAACCACGAGGCCCTGCGCCAGGCCAAGCAGGAGATGAACGAGTCCCGA
CGCCAGATCCAGAGTCTAACGTGCGAGGTGGACGGGCTGCGCGGCACGgt
gagtacgaagctgcgcgctcgggcccggggagcggacgatgaaatgttct
gcaactggccccttccactctcctaccccagAACGAGGCGCTGCTCAGGC
AGTTGAGAGAGCTGGAGGAGCAGTTCGCCCTGGAGGCGGGGGGCTACCAG
GCGGGCGCTGCGCGGCTCGAGGAGGAGCTGCGACAGCTAAAAGAGGAGAT
GGCGCGGCACCTGAGGGAGTACCAGGAGCTCCTCAACGTCAAGATGGCCC
TGGACATCGAGATCGCCACCTACCGCAAGCTGCTGGAGGGCGAGGAGAGC
CGgtgagggtggagctgctggggcggggcagggcggggtcgggactgggc
cgggcagggcggggcctgggcaggggcgctgacaacttgcttcgcctcta
gGATCTCCGTGCCCGTCCATTCTTTTGCCTCCTTAAATATAAAGACGACT
Ggtgagtctggcttacagcctgtacctctccttgtccacttctgccctcc
tcgggctcttgctctggctcacctcagggatctcttctccccacggaact
tctgggtcctggcctgtacccacccctactcctcaaacccgcccctcccc
tgccctcagggatagcagtgggagcctaagaggagagattcccacgcctt
cccccttgaaccctttatcctgctttcttcagTGCCTGAGGTGGAGCCTC
CCCAGGACAGCCACAGCCGGAAGACGGTTCTGATCAAGACCATTGAGACC
CGGAATGGGGAGgtgaggcaggtcccctaatgccaggaccccaccatttc
ccatattatttctgagccccagcctggctgtcgctaatcttacttagggt
gggtaattcttggggagagacagagaaggagacagagaacgtggatagat
aaccaggagcctctgctggttaccccagggagtggggctctatgatccaa
aggagtaggatggagggtggcgctgaatggcttgtgcccatcatatgccc
tgtccccagcagGTGGTGACAGAGTCCCAGAAGGAGCAGCGCAGTGAGCT
GGACAAGTCTTCTGCCCACAGTTACTGAaccccttggtccggagccttga
ctctgccctaggcctgctcaaagcccaaaccctaagaccactcctgaatt
gtctcctctccctctgcatgtgtctaaaaggtggtaccaggcatcccttt
cctggcttatggccaagccctacccggccagcagtcgctgggcctctccc
tgccctgacacttgatgtgacctatgtgcttcccttttcatgtcccgata
agaagccaatgatcccccctcaggacaaatctactccagccacgatgaga
agtgggtgagccagggtctgagtttcacatttgaaccaaataaaatgctg
tcaagagaaaactctccagtgca

TABLE 2

Per 3, 4 cDNA
Intron 3 position: 703-1063 bp (361 bp); Intron 4
position: 1232-1322 (91 bp) (underlined) (early
stop codon bolded)

(SEQ ID NO: 2)

cctcgcagcggtctgcggctccttcccagcccccggcctagctctgcgaa
cggtgactgcccatccttggccgcaATGAGCCACCACCCGTCGGGCCTCC
GGGCCGGCTTCAGCTCCACCTCATACCGCCGTACCTTCGGTCCACCGCCC
TCACTATCCCCCGGGGCCTTCTCCTACTCGTCCAGCTCCCGCTTCTCCAG
CAGCCGCCTGCTGGGCTCCGCGTCCCCGAGCTCCTCGGTGCGCCTGGGCA
GCTTCCGTAGCCCCCGAGCGGGAGCGGGCGCCCTCCTGCGCCTGCCCTCG
GAGCGCCTCGACTTCTCCATGGCCGAGGCCCTCAACCAGGAGTTCCTGGC
CACGCGCAGCAACGAGAAGCAGGAGCTGCAGGAGCTCAACGACCGCTTCG
CCAACTTCATCGAGAAGGTACGCTTTCTGGAGCAGCAGAACGCGGCCCTG
CGCGGGGAGCTGAGCCAAGCCCGGGGCCAGGAGCCGGCGCGCGCCGACCA
GCTGTGCCAGCAGGAGCTGCGCGAGCTGCGGCGAGAGCTGGAGCTGTTGG
GCCGCGAGCGTGACCGGGTGCAGGTGGAGCGCGACGGGCTGGCGGAGGAC
CTGGCGGCGCTCAAGCAGAGGTTGGAGGAGGAGACGCGCAAGCGGGAGGA
CGCGGAGCACAACCTCGTGCTCTTCCGCAAGGACGTGGACGATGCCACTC
TGTCCCGCCTGGAACTAGAGCGCAAGATTGAGTCTCTGATGGATGAGATT
GAGTTCCTCAAGAAGCTGCACGAGGAGGTAAGTGGGCCCGGTATCAGGGG
CGGTTTCTGAGGTTGTGGGGTGGTCTCGCTGGAGCTGGCGGGTGGAGCGG
AGGCATCGCCCTGGGGATCAGGACGATGCTGGGTAGACGCAGCCCCTCCA
CCCTAGTCTACAGGTGGTTAGACTCCCACCCTTGCGCCACCTGGCGGCGG
GCAGCGGGGCTGTACCTCCGAAACCTGGCCTCTGGTCTCGCGCCCGCGGG
GGCGCAGGGCTGTACGCCCTGCCCTCCCTGGCGCCCACTTCTGTTCGTTC
AAGCGTTTCTTCTCTTTTCTGTGCACGAACTGCGTGGCCCGCGTGGAATT
TGCGCCGCTGTCCATCCTCTGCCTGCTCCCGGCCGTAGGAGCTGCGAGAC
CTGCAGGTGAGTGTGGAGAGCCAGCAGGTGCAGCAGGTGGAGGTGGAAGC
CACGGTGAAGCCCGAGCTGACGGCAGCGCTGAGGGACATCCGCGCGCAGT
ACGAGAGCATCGCCGCGAAGAACCTGCAGGAGGCGGAGGAGTGGTACAAG
TCCAAGGTGCAAGAGCCGGGAGGGCCTGCGAGGCGGGACGCTGGGGTGGT
GTCGCGCGTCCCAGCCGACTAAAGCCTGGGTTACCCCCACTTCTCAGTAC
GCGGACCTGTCCGACGCTGCCAACCGGAACCACGAGGCCCTGCGCCAGGC
CAAGCAGGAGATGAACGAGTCCCGACGCCAGATCCAGAGTCTAACGTGCG
AGGTGGACGGGCTGCGCGGCACGAACGAGGCGCTGCTCAGGCAGTTGAGA
GAGCTGGAGGAGCAGTTCGCCCTGGAGGCGGGGGGCTACCAGGCGGGCGC
TGCGCGGCTCGAGGAGGAGCTGCGACAGCTAAAAGAGGAGATGGCGCGGC
ACCTGAGGGAGTACCAGGAGCTCCTCAACGTCAAGATGGCCCTGGACATC
GAGATCGCCACCTACCGCAAGCTGCTGGAGGGCGAGGAGAGCCGGATCTC
CGTGCCCGTCCATTCTTTTGCCTCCTTAAATATAAAGACGACTGTGCCTG
AGGTGGAGCCTCCCCAGGACAGCCACAGCCGGAAGACGGTTCTGATCAAG
ACCATTGAGACCCGGAATGGGGAGGTGGTGACAGAGTCCCAGAAGGAGCA
GCGCAGTGAGCTGGACAAGTCTTCTGCCCACAGTTACTGAaccccttggt
ccggagccttgactctgccctaggcctgctcaaagcccaaaccctaagac
cactcctgaattgtctcctctccctctgcatgtgtctaaaaggtggtacc
aggcatccctttcctggcttatggccaagccctacccggccagcagtcgc
tgggcctctccctgccctgacacttgatgtgacctatgtgcttccctttt
catgtcccgataagaagccaatgatcccccctcaggacaaatctactcca
gccacgatgagaagtgggtgagccagggtctgagtttcacatttgaacca
aataaaatgctgtcaagagaaaactctccagtgca
First 30 bp of intron 3:
GTAAGTGGGCCCGGTATCAGGGGCGGTTTC
(SEQ ID NO: 3) - (Position 702-732 bp from first
ATG)
Translation from intron 3:
10 aa sequence V-S-G-P-G-I-R-G-G-F (SEQ ID NO: 4)

TABLE 3
Per 28 protein sequence

| | |
|---|---|
| 1 | M S H H P S G L R A G F S S T S Y R R T |
| 1 | ATGAGCCACCACCCGTCGGGCCTCCGGGCCGGCTTCAGCTCCACCTCATACCGCCGTACC |
| 21 | F G P P P S L S P G A F S Y S S S S R F |
| 61 | TTCGGTCCACCGCCCTCACTATCCCCCGGGGCCTTCTCCTACTCGTCCAGCTCCCGCTTC |
| 41 | S S S R L L G S A S P S S S V R L G S F |
| 121 | TCCAGCAGCCGCCTGCTGGGCTCCGCGTCCCCGAGCTCCTCGGTGCGCCTGGGCAGCTTC |
| 61 | R S P R A G A G A L L R L P S E R L D F |
| 181 | CGTAGCCCCCGAGCGGGAGCGGGCGCCCTCCTGCGCCTGCCCTCGGAGCGCCTCGACTTC |
| 81 | S M A E A L N Q E F L A T R S N E K Q E |
| 241 | TCCATGGCCGAGGCCCTCAACCAGGAGTTCCTGGCCACGCGCAGCAACGAGAAGCAGGAG |
| 101 | L Q E L N D R F A N F I E K V R F L E Q |
| 301 | CTGCAGGAGCTCAACGACCGCTTCGCCAACTTCATCGAGAAGGTACGCTTTCTGGAGCAG |
| 121 | Q N A A L R G E L S Q A R G Q E P A R A |
| 361 | CAGAACGCGGCCCTGCGCGGGGAGCTGAGCCAAGCCCGGGGCCAGGAGCCGGCGCGCGCC |
| 141 | D Q L C Q Q E L R E L R R E L E L L G R |
| 421 | GACCAGCTGTGCCAGCAGGAGCTGCGCGAGCTGCGGCGAGAGCTGGAGCTGTTGGGCCGC |
| 161 | E R D R V Q V E R D G L A E D L A A L K |
| 481 | GAGCGTGACCGGGTGCAGGTGGAGCGCGACGGGCTGGCGGAGGACCTGGCGGCGCTCAAG |
| 181 | Q R L E E E T R K R E D A E H N L V L F |
| 541 | CAGAGGTTGGAGGAGGAGACGCGCAAGCGGGAGGACGCGGAGCACAACCTCGTGCTCTTC |
| 201 | R K D V D D A T L S R L E L E R K I E S |
| 601 | CGCAAGGACGTGGACGATGCCACTCTGTCCCGCCTGGAACTAGAGCGCAAGATTGAGTCT |
| 221 | L M D E I E F L K K L H E E V S G P G I |
| 661 | CTGATGGATGAGATTGAGTTCCTCAAGAAGCTGCACGAGGAGGTAAGTGGGCCCGGTATC |
| 241 | R G G F - SEQ ID NO: 5 |
| 721 | AGGGGCGGTTTCTGA |

Peptide sequence used to make Per 3,4 (Per 28) antibody located
at 235-244 (underlined and bolded above).

TABLE 4

siRNAs
The sequences provided are short hairpin RNA
(shRNA). These sequences make a tight hairpin turn
that can be used to silence gene expression via
RNA interference.
The forward and reverse primers are mixed to-
gether, they anneal and are then cloned into an
siRNA expression vector. This is then transfected
into cells, the targeting RNA forms a loop that is
then cleaved by Dicer. The resulting fragments act
as siRNA.
The bolded sequences are the restriction sites;
the underlined sequence is the sequence targeted
in the RNA.

Per3, 4 siRNA-1-F:

(SEQ ID NO: 19)

TCGAGGTCTACAGGTGGTTAGACTGGAATTCGAGTCTAACCACCTGTAG
ACTTTTT
Corresponds to 831-849 bp in SEQ ID NO: 2
Per3, 4 siRNA-1-R:

(SEQ ID NO: 20)

CTAGAAAAAGTCTACAGGTGGTTAGACTCGAATTCCAGTCTAACCACCT
GTAGACC
Per3, 4 siRNA-2-F:

(SEQ ID NO: 21)

TCGAGTTCTGTTCGTTCAAGCGTTGGAATTCGAACGCTTGAACGAACAG
AATTTTT
Corresponds to 964-982 bp SEQ ID NO: 2
Per3, 4 siRNA-2-R:

(SEQ ID NO: 22)

CTAGAAAAATTCTGTTCGTTCAAGCGTTCGAATTCCAACGCTTGAACGA
ACAGAAC
NFL-60 siRNA-F:

(SEQ ID NO: 23)

TCGAGCTCAAGTCCATCCGCGACCGGAATTCGGGTCGCGGATGGACTT
GAGTTTTT
The underlined portion corresponds to 247-265
bp of SEQ ID: 7
NFL-60 siRNA-R:

(SEQ ID NO: 24)

CTAGAAAAACTCAAGTCCATCCGCGACCCGAATTCCGGTCGCGGATGG
ACTTGAGC
NFL-57 si-RNA-F:

(SEQ ID NO: 25)

TCGAGCAAAGGCGCCAAGAACACCGGAATTCGGGTGTTCTTGGCGCCT
TTGTTTTT
The underlined portion corresponds to 588-606
bp of SEQ ID NO: 9
NFL-57 siRNA-R:

(SEQ ID NO: 26)

CTAGAAAAACAAAGGCGCCAAGAACACCCGAATTCCGGTGTTCTTGGC
GCCTTTGC

TABLE 5

Summary: NFL-60 caused by in-frame deletion of
171 bp at positions 255 bp to 426 bp. This cor-
responds to a 57 aa deletion generating a new
sequence NDLKSIRDLR (SEQ ID NO: 8) at aa position
81-90.
NFL-60: 171 bp in-frame deletion at positions
255 bp to 426 bp from the first ATG. Shown as
underlined
Human NFL Genomic sequence

(SEQ ID NO: 6)

gcacacagccatccatcctcccccttccctctctcccctgtcctctctct
ccgggctcccaccgccgccgcgggccggggagccaccggccgccaccATG
AGTTCCTTCAGCTACGAGCCGTACTACTCGACCTCCTACAAGCGGCGCTA
CGTGGAGACGCCCCGGGTGCACATCTCCAGCGTGCGCAGCGGCTACAGCA
CCGCACGCTCAGCTTACTCCAGCTACTCGGCGCCGGTGTCTTCCTCGCTG
TCCGTGCGCCGCAGCTACTCCTCCAGCTCTGGATCGTTGATGCCCAGTCT
GGAGAACCTCGACCTGAGCCAGGTAGCCGCCATCAGCAACGACCTCAAGT
CCATCCGCACGCAGGAGAAGGCGCAGCTCCAGGACCTCAATGACCGCTTC
GCCAGCTTCATCGAGCGCGTGCACGAGCTGGAGCAGCAGAACAAGGTCCT
GGAAGCCGAGCTGCTGGTGCTGCGCCAGAAGCACTCCGAGCCATCCCGCT
TCCGGGCGCTGTACGAGCAGGAGATCCGCGACCTGCGCCTGGCGGCGGAA
GATGCCACCAACGAGAAGCAGGCGCTCCAGGGCGAGCGCGAAGGGCTGGA
GGAGACCCTGCGCAACCTGCAGGCGCGCTATGAAGAGGAGGTGCTGAGCC
GCGAGGACGCCGAGGGCCGGCTGATGGAAGCGCGCAAAGGCGCCGACGAG
GCGGCGCTCGCTCGCGCCGAGCTCGAGAAGCGCATCGACAGCTTGATGGA
CGAAATCTCTTTTCTGAAGAAAGTGCACGAAGAGGAGATCGCCGAACTGC
AGGCGCAGATCCAGTACGCGCAGATCTCCGTGGAGATGGACGTGACCAAG
CCCGACCTTTCCGCCGCGCTCAAGGACATCCGCGCGCAGTACGAGAAGCT
GGCCGCCAAGAACATGCAGAACGCTGAGGAATGGTTCAAGAGCCGCTTCA
CCGTGCTGACCGAGAGCGCCGCCAAGAACACCGACGCCGTGCGCGCCGCC
AAGGACGAGGTGTCCGAGAGCCGTCGTCTGCTCAAGGCCAAGACCCTGGA
AATCGAAGCATGCCGGGGCATGAATGAAGCGCTGGAGAAGCAGCTGCAGG
AGCTGGAGGACAAGCAGAACGCCGACATCAGCGCTATGCAGgtgcggcac
ggccagaaacacaggggggcgggggactcgagcaagggggggagttggtg
cgcccagaaagtggagaccaggggtggtgcggctgcacgcagctcttagg
gatagggcttggctccttggccactgtgtgggaggggtggggcacttgaa
gggcgtgagtgcgggcgccactgtagtctgggagtgtgctccgtgctgct
gcaccggcgttccgcattaaagctgcccagcccttgttgggtgggggagg
ggaagacgtgggaattgggcgttgcctccggcctgcagtgagatcagctc
tctactgacctgcattaaccacaagttactttgcagcaactatcggatca
tctagttaataaatagtagagtgaacaactctcaattaattctgaaggat
tactgtgaccagcatgctttatgactagttttaccaaccactcccttcct
ttatttagtaggtagacaggaaaatagtcaacattgttttaggtagttaa
ctagtgatgttcatagtaaaccatttccttttaccttttttttttctttt
tttctttatgtgtaaaatcttctacaacatttctgtttaaacatctccat
cttctggggagtagaaaaaatacaattttaaaaagatctccattttaaaa
catctccatcttctggggagtagaaatttttttcttcttctggggagtag
aaaaaataatttagatacataggaaatatttcatagaaaataattttttt
cttttttttgtttacatctggctattttcttctcataaagaaaggcatta
gtttcctggcatgtaacccagctaaagaagagttaatcagtgaatgagag
acacagtttttctatcaacttagtctgtttgcatgcattttatgatgatc
attaaacagtattaagtaaagaaacagaagaacagaattttcgtccatct
tttttttcatctcaggcttcatgaacttgggtattttaggcatgaaggtt
tttcaaaagatacaggaagttattctaggagagattttatcaaaggtgtg
caccttgattttaatcgaaactaggcctttgcaactacactacagtaaaa
taatagaagggatttatgctcggattttttttttgttttatttttgtctt
caaacagGACACGATCAACAAATTAGAAAATGAATTGAGGACCACAAAGA
GTGAAATGGCACGATACCTAAAAGAATACCAAGACCTCCTCAACGTGAAG
ATGGCTTTGGATATTGAGATTGCAGCTTACAGgtgaaaatagaggggcaa
agacagcagccattaaaccttaggaagaaaatcagatcccatttaaagtt
atgttggatcagaaaccttcaataatagtccttttgaaataatgaagtgt
tagtttttggcttcttccaagaagagggtatttagatatataagaattta
accctgtaattaggagtcctgtttttatcttgtcattacactttaaatct
aataggatgatttatttatattttttctggtctccatcaaaagatcccca
ggcattaagtattgataaatcccagccctgctcctgcttgcctttgtgtt
tagggtactcagagcaagttgtgaaacacaggtgttttttaacctcacct
tgcatctgcatccccagGAAACTCTTGGAAGGCGAGGAGACCCGACTCAG
TTTCACCAGCGTGGGAAGCATAACCAGTGGCTACTCCCAGAGCTCCCAGG
TCTTTGGCCGATCTGCCTACGGCGGTTTACAGACCAGCTCCTATCTGATG
TCCACCCGCTCCTTCCCGTCCTACTACACCAGCCATGTCCAAGAGGAGCA
GATCGAAGTGGAGGAAACCATTGAGGCTGCCAAGGCTGAGGAAGCCAAGG
ATGAGGCCCCCCTCTGAAGGAGAAGCCGAGGAGGAGGAGAAGGACAAGGA
AGAGGCCGAGGAAGAGGAGGCAGCTGAAGAGGAAGAAGgtatgataagaa
aaaacccctgcaacttcaagtgtaaactgggtgtggagatttgttaggag
gtggataagacaaatgaagccttgctcatttattcatatatgacattaga
atcataaataaattttctgtttgtttagcaaaactttcctaaggcatcta
ctctgaatgaggtgattggtcaaaattttcattttttaatataatcattt
aacacagcaggttggtgtcctaaagaacaaaaatagataccagacacata
atgaaagaaatattgaggttaagtcttggagaggagcagagcttcccata
cctagaagtgatctcattcgatttaaatatgtgttcagtggcaaattatt
catggcaagctttgtctgttacatgtgcttttggagagagtggagctggg
aggttttggtagcattctgacagttgtgtttgcaaataaaacctttgcag
acatgttttgactggacttaccctggatttgcattttgtacattttcttt
ttatgttaaagCTGCCAAGGAAGAGTCTGAAGAAGCAAAAGAAGAAGAAG
AAGGAGGTGAAGGTGAAGAAGGAGAGGAAACCAAAGAAGCTGAAGAGGAG
GAGAAGAAAGTTGAAGGTGCTGGGGAGGAACAAGCAGCTAAGAAGAAAGA
TTGAACCCCCATTTCCTTAATTATTTCAGGAATAATTCTCCCGAAATCAG
GTCAACCCCATCACCAACCAACCAACCAGTTGAGTTCCAGATTCTATGTG
AATTAAAAAGTCAATATATGTATAATTCTGAGATGACTTAGGTTGGACAT
TCAATGTTGTGCTATGAATTTCCTCTTTATGCAGAGTATCTCTTTGCTTG
CAGAGTGGCTTTCTGGCTTGCTCCCAGCCTGTGCATGGACCACGCTTATG
AGTTCAGGATCTACGGCAATGTGAATCATTCAGATGTTTACAATAAAAAA
CACCACATGACTAAATGAATTCACTAATGTTAATCTTAAACTTCATGGAA
AAATAGTCCTTTGAACCTTCGGTGGTTAGCAATTAAAGACCCTGAGTTAT
GTGCAATAAATAGTAAATAAAGTTATACCGAA

TABLE 6

NFL-60 cDNA and protein sequence. Sequence used to make
NFL-60 antibody underlined.

SEQ ID NO: 7

| | |
|---|---|
| 1 | M S S F S Y E P Y Y S T S Y K R R Y V E |
| 1 | ATGAGTTCCTTCAGCTACGAGCCGTACTACTCGACCTCCTACAAGCGGCGCTACGTGGAG |
| 21 | T P R V H I S S V R S G Y S T A R S A Y |
| 61 | ACGCCCCGGGTGCACATCTCCAGCGTGCGCAGCGGCTACAGCACCGCACGCTCAGCTTAC |
| 41 | S S Y S A P V S S S L S V R R S Y S S S |
| 121 | TCCAGCTACTCGGCGCCGGTGTCTTCCTCGCTGTCCGTGCGCCGCAGCTACTCCTCCAGC |
| 61 | S G S L M P S L E N L D L S Q V A A I S |
| 181 | TCTGGATCGTTGATGCCCAGTCTGGAGAACCTCGACCTGAGCCAGGTAGCCGCCATCAGC |
| 81 | N D L K S I R D L R L A A E D A T N E K |
| 241 | AACGACCTCAAGTCCATCCGCGACCTGCGCCTGGCGGCGGAAGATGCCACCAACGAGAAG |
| 101 | Q A L Q G E R E G L E E T L R N L Q A R |
| 301 | CAGGCGCTCCAGGGCGAGCGCGAAGGGCTGGAGGAGACCCTGCGCAACCTGCAGGCGCGC |
| 121 | Y E E E V L S R E D A E G R L M E A R K |
| 361 | TATGAAGAGGAGGTGCTGAGCCGCGAGGACGCCGAGGGCCGGCTGATGGAAGCGCGCAAA |
| 141 | G A D E A A L A R A E L E K R I D S L M |
| 421 | GGCGCCGACGAGGCGGCGCTCGCTCGCGCCGAGCTCGAGAAGCGCATCGACAGCTTGATG |
| 161 | D E I S F L K K V H E E E I A E L Q A Q |
| 481 | GACGAAATCTCTTTTCTGAAGAAAGTGCACGAAGAGGAGATCGCCGAACTGCAGGCGCAG |
| 181 | I Q Y A Q I S V E M D V T K P D L S A A |
| 541 | ATCCAGTACGCGCAGATCTCCGTGGAGATGGACGTGACCAAGCCCGACCTTTCCGCCGCG |
| 201 | L K D I R A Q Y E K L A A K N M Q N A E |
| 601 | CTCAAGGACATCCGCGCGCAGTACGAGAAGCTGGCCGCCAAGAACATGCAGAACGCTGAG |
| 221 | E W F K S R F T V L T E S A A K N T D A |
| 661 | GAATGGTTCAAGAGCCGCTTCACCGTGCTGACCGAGAGCGCCGCCAAGAACACCGACGCC |
| 241 | V R A A K D E V S E S R R L L K A K T L |
| 721 | GTGCGCGCCGCCAAGGACGAGGTGTCCGAGAGCCGTCGTCTGCTCAAGGCCAAGACCCTG |
| 261 | E I E A C R G M N E A L E K Q L Q E L E |
| 781 | GAAATCGAAGCATGCCGGGGCATGAATGAAGCGCTGGAGAAGCAGCTGCAGGAGCTGGAG |
| 281 | D K Q N A D I S A M Q D T I N K L E N E |
| 841 | GACAAGCAGAACGCCGACATCAGCGCTATGCAGGACACGATCAACAAATTAGAAAATGAA |
| 301 | L R T T K S E M A R Y L K E Y Q D L L N |
| 901 | TTGAGGACCACAAAGAGTGAAATGGCACGATACCTAAAAGAATACCAAGACCTCCTCAAC |
| 321 | V K M A L D I E I A A Y R K L L E G E E |
| 961 | GTGAAGATGGCTTTGGATATTGAGATTGCAGCTTACAGGAAACTCTTGGAAGGCGAGGAG |
| 341 | T R L S F T S V G S I T S G Y S Q S S Q |
| 1021 | ACCCGACTCAGTTTCACCAGCGTGGGAAGCATAACCAGTGGCTACTCCCAGAGCTCCCAG |
| 361 | V F G R S A Y G G L Q T S S Y L M S T R |
| 1081 | GTCTTTGGCCGATCTGCCTACGGCGGTTTACAGACCAGCTCCTATCTGATGTCCACCCGC |
| 381 | S F P S Y Y T S H V Q E E Q I E V E E T |
| 1141 | TCCTTCCCGTCCTACTACACCAGCCATGTCCAAGAGGAGCAGATCGAAGTGGAGGAAACC |
| 401 | I E A A K A E E A K D E P P S E G E A E |
| 1201 | ATTGAGGCTGCCAAGGCTGAGGAAGCCAAGGATGAGCCCCCCTCTGAAGGAGAAGCCGAG |
| 421 | E E E K D K E E A E E E E A A E E E E A |
| 1261 | GAGGAGGAGAAGGACAAGGAAGAGGCCGAGGAAGAGGAGGCAGCTGAAGAGGAAGAAGCT |
| 441 | A K E E S E E A K E E E E G G E G E E G |
| 1321 | GCCAAGGAAGAGTCTGAAGAAGCAAAAGAAGAAGAAGAAGGAGGTGAAGGTGAAGAAGGA |
| 461 | E E T K E A E E E E K K V E G A G E E Q |
| 1381 | GAGGAAACCAAAGAAGCTGAAGAGGAGGAGAAGAAAGTTGAAGGTGCTGGGGAGGAACAA |
| 481 | A A K K K D - |
| 1441 | GCAGCTAAGAAGAAAGATTGA |

Sequence used to make NFL-60 antibody underlined:

(SEQ ID NO: 8)

N D L K S I R D L R

TABLE 7

Summary: NFL-57 caused by in-frame deletion of
279 bp at positions 597 bp to 875 bp. This cor-
responds to a 93 aa deletion generating a new
sequence ARKGAKNTDA (SEQ ID NO: 10) at aa
position 195-204.
NFL-57: 279 bp in-frame deletion at positions
597 bp to 875 bp from the first ATG. Shown as
underlined.
Human NFL Genomic sequence

(SEQ ID NO: 6)

gcacacagccatccatcctcccccttccctctctcccctgtcctctctct
ccgggctcccaccgccgccgcgggccggggagccaccggccgccaccATG
AGTTCCTTCAGCTACGAGCCGTACTACTCGACCTCCTACAAGCGGCGCTA
CGTGGAGACGCCCCGGGTGCACATCTCCAGCGTGCGCAGCGGCTACAGCA
CCGCACGCTCAGCTTACTCCAGCTACTCGGCGCCGGTGTCTTCCTCGCTG
TCCGTGCGCCGCAGCTACTCCTCCAGCTCTGGATCGTTGATGCCCAGTCT
GGAGAACCTCGACCTGAGCCAGGTAGCCGCCATCAGCAACGACCTCAAGT
CCATCCGCACGCAGGAGAAGGCGCAGCTCCAGGACCTCAATGACCGCTTC
GCCAGCTTCATCGAGCGCGTGCACGAGCTGGAGCAGCAGAACAAGGTCCT
GGAAGCCGAGCTGCTGGTGCTGCGCCAGAAGCACTCCGAGCCATCCCGCT
TCCGGGCGCTGTACGAGCAGGAGATCCGCGACCTGCGCCTGGCGGCGGAA
GATGCCACCAACGAGAAGCAGGCGCTCCAGGGCGAGCGCGAAGGGCTGGA
GGAGACCCTGCGCAACCTGCAGGCGCGCTATGAAGAGGAGGTGCTGAGCC
GCGAGGACGCCGAGGGCCGGCTGATGGAAGCGCGCAAAGGCGCCGACGAG
GCGGCGCTCGCTCGCGCCGAGCTCGAGAAGCGCATCGACAGCTTGATGGA
CGAAATCTCTTTTCTGAAGAAAGTGCACGAAGAGGAGATCGCCGAACTGC
AGGCGCAGATCCAGTACGCGCAGATCTCCGTGGAGATGGACGTGACCAAG
CCCGACCTTTCCGCCGCGCTCAAGGACATCCGCGCGCAGTACGAGAAGCT
GGCCGCCAAGAACATGCAGAACGCTGAGGAATGGTTCAAGAGCCGCTTCA
CCGTGCTGACCGAGAGCGCCGCCAAGAACACCGACGCCGTGCGCGCCGCC
AAGGACGAGGTGTCCGAGAGCCGTCGTCTGCTCAAGGCCAAGACCCTGGA
AATCGAAGCATGCCGGGGCATGAATGAAGCGCTGGAGAAGCAGCTGCAGG
AGCTGGAGGACAAGCAGAACGCCGACATCAGCGCTATGCAGgtgcggcac
ggccagaaacacaggggggcgggggactcgagcaagggggggagttggtg
cgcccagaaagtggagaccaggggtggtgcggctgcacgcagctcttagg
gatagggcttggctccttggccactgtgtgggaggggtggggcacttgaa
gggcgtgagtgcgggcgccactgtagtctgggagtgtgctccgtgctgct
gcaccggcgttccgcattaaagctgcccagcccttgttgggtgggggagg
ggaagacgtgggaattgggcgttgcctccggcctgcagtgagatcagctc
tctactgacctgcattaaccacaagttactttgcagcaactatcggatca
tctagttaataaatagtagagtgaacaactctcaattaattctgaaggat
tactgtgaccagcatgctttatgactagttttaccaaccactcccttcct
ttatttagtaggtagacaggaaaatagtcaacattgttttaggtagttaa
ctagtgatgttcatagtaaaccatttccttttaccttttttttttctttt
tttctttatgtgtaaaatcttctacaacatttctgtttaaacatctccat
cttctggggagtagaaaaaatacaattttaaaaagatctccattttaaaa
catctccatcttctggggagtagaaatttttttcttcttctggggagtag
aaaaaataatttagatacataggaaatatttcatagaaaataattttttt
cttttttttgtttacatctggctattttcttctcataaagaaaggcatta
gtttcctggcatgtaacccagctaaagaagagttaatcagtgaatgagag
acacagtttttctatcaacttagtctgtttgcatgcattttatgatgatc
attaaacagtattaagtaaagaaacagaagaacagaattttcgtccatct
tttttttcatctcaggcttcatgaacttgggtattttaggcatgaaggtt
tttcaaaagatacaggaagttattctaggagagattttatcaaaggtgtg
caccttgattttaatcgaaactaggcctttgcaactacactacagtaaaa
taatagaagggatttatgctcggattttttttttgttttatttttgtctt
caaacagGACACGATCAACAAATTAGAAAATGAATTGAGGACCACAAAGA
GTGAAATGGCACGATACCTAAAAGAATACCAAGACCTCCTCAACGTGAAG
ATGGCTTTGGATATTGAGATTGCAGCTTACAGgtgaaaatagaggggcaa
agacagcagccattaaaccttaggaagaaaatcagatcccatttaaagtt
atgttggatcagaaaccttcaataatagtccttttgaaataatgaagtgt
tagtttttggcttcttccaagaagagggtatttagatatataagaattta
accctgtaattaggagtcctgtttttatcttgtcattacactttaaatct
aataggatgatttatttatattttttctggtctccatcaaaagatcccca
ggcattaagtattgataaatcccagccctgctcctgcttgcctttgtgtt
tagggtactcagagcaagttgtgaaacacaggtgttttttaacctcacct
tgcatctgcatccccagGAAACTCTTGGAAGGCGAGGAGACCCGACTCAG
TTTCACCAGCGTGGGAAGCATAACCAGTGGCTACTCCCAGAGCTCCCAGG
TCTTTGGCCGATCTGCCTACGGCGGTTTACAGACCAGCTCCTATCTGATG
TCCACCCGCTCCTTCCCGTCCTACTACACCAGCCATGTCCAAGAGGAGCA
GATCGAAGTGGAGGAAACCATTGAGGCTGCCAAGGCTGAGGAAGCCAAGG
ATGAGcCCCCCCTCTGAAGGAGAAGCCGAGGAGGAGGAGAAGGACAAGGA
AGAGGCCGAGGAAGAGGAGGCAGCTGAAGAGGAAGAAGgtatgataagaa
aaaacccctgcaacttcaagtgtaaactgggtgtggagatttgttaggag
gtggataagacaaatgaagccttgctcatttattcatatatgacattaga
atcataaataaattttctgtttgtttagcaaaactttcctaaggcatcta
ctctgaatgaggtgattggtcaaaattttcattttttaatataatcattt
aacacagcaggttggtgtcctaaagaacaaaaatagataccagacacata
atgaaagaaatattgaggttaagtcttggagaggagcagagcttcccata
cctagaagtgatctcattcgatttaaatatgtgttcagtggcaaattatt
catggcaagctttgtctgttacatgtgcttttggagagagtggagctggg
aggttttggtagcattctgacagttgtgtttgcaaataaaacctttgcag
acatgttttgactggacttaccctggatttgcattttgtacattttCttt
ttatgttaaagCTGCCAAGGAAGAGTCTGAAGAAGCAAAAGAAGAAGAAG
AAGGAGGTGAAGGTGAAGAAGGAGAGGAAACCAAAGAAGCTGAAGAGGAG
GAGAAGAAAGTTGAAGGTGCTGGGGAGGAACAAGCAGCTAAGAAGAAAGA
TTGAACCCCCATTTCCTTAATTATTTCAGGAATAATTCTCCCGAAATCAG
GTCAACCCCATCACCAACCAACCAACCAGTTGAGTTCCAGATTCTATGTG
AATTAAAAAGTCAATATATGTATAATTCTGAGATGACTTAGCTTGGACAT
TCAATGTTGTGCTATGAATTTCCTCTTTATGCAGAGTATCTGTTTGCTTG
CAGAGTGGCTTTCTGGCTTGCTGCCAGCCTGTGCATGGACCACGCTTATG
AGTTCAGGATCTACGGCAATGTGAATCATTCAGATGTTTACAATAAAAAA
CACCACATGAGTAAATGAATTCACTAATGTTAATGTTAAACTTCATGGAA
AAATAGTCCTTTGAACCTTCGGTGGTTAGCAATTAAAGACCCTGAGTTAT
GTGCAATAAATAGTAAATAAAGTTATACCGAA

TABLE 8

NFL-57 cDNA and protein sequence. Sequence used to make
NFL-57 antibody shown as underlined.

| | |
|---|---|
| 1 | atgagttccttcagctacgagccgtactactcgacctcctacaagcggcgctacgtggag |
| 1 | M S S F S Y E P Y Y S T S Y K R R Y V E |
| 61 | acgccccgggtgcacatctccagcgtgcgcagcggctacagcaccgcacgctcagcttac |
| 21 | T P R V H I S S V R S G Y S T A R S A Y |
| 121 | tccagctactcggcgccggtgtcttcctcgctgtccgtgcgccgcagctactcctccagc |
| 41 | S S Y S A P V S S S L S V R R S Y S S S |
| 181 | tctggatcgttgatgcccagtctggagaacctcgacctgagccaggtagccgccatcagc |
| 61 | S G S L M P S L E N L D L S Q V A A I S |
| 241 | aacgacctcaagtccatccgcacgcaggagaaggcgcagctccaggacctcaatgaccgc |
| 81 | N D L K S I R T Q E K A Q L Q D L N D R |
| 301 | ttcgccagcttcatcgagcgcgtgcacgagctggagcagcagaacaaggtcctggaagcc |
| 101 | F A S F I E R V H E L E Q Q N K V L E A |
| 361 | gagctgctggtgctgcgccagaagcactccgagccatcccgcttccgggcgctgtacgag |
| 121 | E L L V L R Q K H S E P S R F R A L Y E |
| 421 | caggagatccgcgacctgcgcctggcggcggaagatgccaccaacgagaagcaggcgctc |
| 141 | Q E I R D L R L A A E D A T N E K Q A L |
| 481 | cagggcgagcgcgaagggctggaggagaccctgcgcaacctgcaggcgcgctatgaagag |
| 161 | Q G E R E G L E E T L R N L Q A R Y E E |
| 541 | gaggtgctgagccgcgaggacgccgagggccggctgatggaagcgcgcaaaggcgccaag |
| 181 | E V L S R E D A E G R L M E A R K G A K |
| 601 | aacaccgacgccgtgcgcgccgccaaggacgaggtgtccgagagccgtcgtctgctcaag |
| 201 | N T D A V R A A K D E V S E S R R L L K |
| 661 | gccaagaccctggaaatcgaagcatgccggggcatgaatgaagcgctggagaagcagctg |
| 221 | A K T L E I E A C R G M N E A L E K Q L |
| 721 | caggagctggaggacaagcagaacgccgacatcagcgttatgcaggacacgatcaacaaa |
| 241 | Q E L E D K Q N A D I S V M Q D T I N K |
| 781 | ttagaaaatgaattgaggaccacaaagagtgaaatggcacgatacctaaaagaataccaa |
| 261 | L E N E L R T T K S E M A R Y L K E Y Q |
| 841 | gacctcctcaacgtgaagatggctttggatattgagattgcagcttacaggaaactcttg |
| 281 | D L L N V K M A L D I E I A A Y R K L L |
| 901 | gaaggcgaggagacccgactcagtttcaccagcgtgggaagcataaccagtggctactcc |
| 301 | E G E E T R L S F T S V G S I T S G Y S |
| 961 | cagagctcccaggtctttggccgatctgcctacggcggtttacagaccagctcctatctg |
| 321 | Q S S Q V F G R S A Y G G L Q T S S Y L |
| 1021 | atgtccacccgctccttcccgtcctactacaccagccatgtccaagaggagcagatcgaa |
| 341 | M S T R S F P S Y Y T S H V Q E E Q I E |
| 1081 | gtggaggaaaccattgaggctgccaaggctgaggaagccaaggatgagcccccctctgaa |
| 361 | V E E T I E A A K A E E A K D E P P S E |
| 1141 | ggagaagccgaggaggaggagaaggacaaggaagaggccgaggaagaggaggcagctgaa |
| 381 | G E A E E E E K D K E E A E E E E A A E |
| 1201 | gaggaagaagctgccaaggaagagtctgaagaagcaaaagaagaagaagaaggaggtgaa |
| 401 | E E E A A K E E S E E A K E E E E G G E |
| 1261 | ggtgaagaaggagaggaaaccaaagaagctgaagaggaggagaagaaagttgaaggtgct |
| 421 | G E E G E E T K E A E E E E K K V E G A |
| 1321 | ggggaggaacaagcagctaagaagaaagattga |
| 441 | G E E Q A A K K K D - |

TABLE 9

Primer sequences

| | | | | SEQ |
|---|---|---|---|---|
| | | | | ID |
| Name | Sequence | Location | Length | NO: |
| F1 | 5′-GTGGACGATGCCACTCTGTC-3′ | Exon 3 | 701 | 11 |
| R1 | 5′-GGTCCGCGTACTGAGAAGTG-3 | Intron | bp & | 12 |
| | | 4/Exon 5 | 339 bp | |
| | | junction | | |
| F2 | 5′-TCGACTTCTCCATGGCCGAG-3′ | Exon 1 | 683 bp | 13 |
| R2 | 5′-CGAGTCCAGAGGCCAGGTTTC-3 | Intron 3 | | 14 |
| PRPH- | 5′-GTGGACGATGCCACTCTGTC-3′ | Exon 3 | 534 bp | 15 |
| Fwd | | | | |
| PRPH- | 5′-CTGGTACTCCCTCAGGTGC-3′ | Exon 6 | | 16 |
| Rvs | | | | |
| ChAT- | 5′-CTCCAATTGGCCTGCTGACGTC | Exon 7 | 233 bp | 17 |
| Fwd | | | | |
| ChAT- | 5′-GGACTTGTCGTACCAGCGATTG- | Exon 8 | | 18 |
| Rvs | 3′ | | | |

TABLE 10
cDNA and Protein Sequence of Per 32 Protein

(1) cDNA Sequence
Intron 4 position: 871-961 from starting codon (91 bp)
(underlined) (early stop codon bolded)

(SEQ ID NO: 27)

cctcgcagcggtctgcggctccttcccagcccccggcctagctctgcgaa
cggtgactgcccatccttggccgcaATGAGCCACCACCCGTCGGGCCTCC
GGGCCGGCTTCAGCTCCACCTCATACCGCCGTACCTTCGGTCCACCGCCC
TCACTATCCCCCGGGGCCTTCTCCTACTCGTCCAGCTCCCGCTTCTCCAG
CAGCCGCCTGCTGGGCTCCGCGTCCCCGAGCTCCTCGGTGCGCCTGGGCA
GCTTCCGTAGCCCCCGAGCGGGAGCGGGCGCCCTCCTGCGCCTGCCCTCG
GAGCGCCTCGACTTCTCCATGGCCGAGGCCCTCAACCAGGAGTTCCTGGC
CACGCGCAGCAACGAGAAGCAGGAGCTGCAGGAGCTCAACGACCGCTTCG
CCAACTTCATCGAGAAGGTACGCTTTCTGGAGCAGCAGAACGCGGCCCTG
CGCGGGGAGCTGAGCCAAGCCCGGGGCCAGGAGCCGGCGCGCGCCGACCA
GCTGTGCCAGCAGGAGCTGCGCGAGCTGCGGCGAGAGCTGGAGCTGTTGG
GCCGCGAGCGTGACCGGGTGCAGGTGGAGCGCGACGGGCTGGCGGAGGAC
CTGGCGGCGCTCAAGCAGAGGTTGGAGGAGGAGACGCGCAAGCGGGAGGA
CGCGGAGCACAACCTCGTGCTCTTCCGCAAGGACGTGGACGATGCCACTC
TGTCCCGCCTGGAACTAGAGCGCAAGATTGAGTCTCTGATGGATGAGATT
GAGTTCCTCAAGAAGCTGCACGAGGAGGAGCTGCGAGACCTGCAGGTGAG
TGTGGAGAGCCAGCAGGTGCAGCAGGTGGAGGTGGAAGCCACGGTGAAGC
CCGAGCTGACGGCAGCGCTGAGGGACATCCGCGCGCAGTACGAGAGCATC
GCCGCGAAGAACCTGCAGGAGGCGGAGGAGTGGTACAAGTCCAAGGTGCA
AGAGCCGGGAGGGCCTGCGAGGCGGGACGCTGGGGTGGTGTCGCGCGTCC
CAGCCGACTAAAGCCTGGGTTACCCCCACTTCTCAGTACGCGGACCTGTC
CGACGCTGCCAACCGGAACCACGAGGCCCTGCGCCAGGCCAAGCAGGAGA
TGAACGAGTCCCGACGCCAGATCCAGAGTCTAACGTGCGAGGTGGACGGG
CTGCGCGGCACGAACGAGGCGCTGCTCAGGCAGTTGAGAGAGCTGGAGGA
GCAGTTCGCCCTGGAGGCGGGGGGCTACCAGGCGGGCGCTGCGCGGCTCG
AGGAGGAGCTGCGACAGCTAAAAGAGGAGATGGCGCGGCACCTGAGGGAG
TACCAGGAGCTCCTCAACGTCAAGATGGCCCTGGACATCGAGATCGCCAC
CTACCGCAAGCTGCTGGAGGGCGAGGAGAGCCGGATCTCCGTGCCCGTCC
ATTCTTTTGCCTCCTTAAATATAAAGACGACTGTGCCTGAGGTGGAGCCT
CCCCAGGACAGCCACAGCCGGAAGACGGTTCTGATCAAGACCATTGAGAC
CCGGAATGGGGAGGTGGTGACAGAGTCCCAGAAGGAGCAGCGCAGTGAGC
TGGACAAGTCTTCTGCCCACAGTTACTGAaccccttggtccggagccttg
Actctgccctaggcctgctcaaagcccaaaccctaagaccactcctgaat
Tgtctcctctccctctgcatgtgtctaaaaggtggtaccaggcatccctt
Tcctggcttatggccaagccctacccggccagcagtcgctgggcctctcc
Ctgccctgacacttgatgtgacctatgtgcttcccttttcatgtcccgat
Aagaagccaatgatcccccctcaggacaaatctactccagccacgatgag
Aagtgggtgagccagggtctgagtttcacatttgaaccaaataaaatgct
Gtcaagagaaaactctccagtgca
(2) Protein Sequence (Unique Sequence generated by
intron is underlined)

(SEQ ID NO: 28)

```
atgagccaccacccgtcgggcctccgggccggcttcagctccacctcataccgccgtacc
  M S H H P S G L R A G F S S T S Y R R T
ttcggtccaccgccctcactatcccccggggccttctcctactcgtccagctcccgcttc
  F G P P P S L S P G A F S Y S S S S R F
tccagcagccgcctgctgggctccgcgtccccgagctcctcggtgcgcctgggcagcttc
  S S S R L L G S A S P S S S V R L G S F
cgtagcccccgagcgggagcgggcgccctcctgcgcctgccctcggagcgcctcgacttc
  R S P R A G A G A L L R L P S E R L D F
tccatggccgaggccctcaaccaggagttcctggccacgcgcagcaacgagaagcaggag
  S M A E A L N Q E F L A T R S N E K Q E
ctgcaggagctcaacgaccgcttcgccaacttcatcgagaaggtacgctttctggagcag
  L Q E L N D R F A N F I E K V R F L E Q
cagaacgcggccctgcgcggggagctgagccaagcccggggccaggagccggcgcgcgcc
  Q N A A L R G E L S Q A R G Q E P A R A
gaccagctgtgccagcaggagctgcgcgagctgcggcgagagctggagctgttgggccgc
  D Q L C Q Q E L R E L R R E L E L L G R
gagcgtgaccgggtgcaggtggagcgcgacgggctggcggaggacctggcggcgctcaag
  E R D R V Q V E R D G L A E D L A A L K
cagaggttggaggaggagacgcgcaagcgggaggacgcggagcacaacctcgtgctcttc
  Q R L E E E T R K R E D A E H N L V L F
cgcaaggacgtggacgatgccactctgtcccgcctggaactagagcgcaagattgagtct
  R K D V D D A T L S R L E L E R K I E S
ctgatggatgagattgagttcctcaagaagctgcacgaggaggagctgcgagacctgcag
  L M D E I E F L K K L H E E E L R D L Q
gtgagtgtggagagccagcaggtgcagcaggtggaggtggaagccacggtgaagcccgag
  V S V E S Q Q V Q Q V E V E A T V K P E
ctgacggcagcgctgagggacatccgcgcgcagtacgagagcatcgccgcgaagaacctg
  L T A A L R D I R A Q Y E S I A A K N L
caggaggcggaggagtggtacaagtccaaggtgcaagagccgggagggcctgcgaggcgg
  Q E A E E W Y K S K V Q E P G G P A R R
gacgctggggtggtgtcgcgcgtcccagccgactaa
  D A G V V S R V P A D -
Unique Sequence generated by retained Intron 4
```

(SEQ ID NO: 29)

V Q E P G G P A R R D A G V V S R V P A D

TABLE 11

The Per 3, 4 ELISA assay has been validated for
detection of Per 3, 4 of ALS cases

| | CSF | ID/Au- | | Dur- | Gen- | Disease | West- | El- |
|---|---|---|---|---|---|---|---|---|
| # | sample | topsy # | Age | ation | der | Type | ern | isa |
| 1 | LP-1 | 1087 | 51 | On- | M | NYD/pos- | +ve | NT |
| | | | | going | sible ALS | | | |
| 2 | LP-1 | 1073 | 64 | On- | M | ALS | −ve | NT |
| | | | | going | | | | |
| 3 | LP-1 | 2454884 | 55 | On- | M | Not ALS | −ve | NT |
| | | | | going | | | | |
| 4 | au- | A07-101 | 60 | 18 | M | Leuko- | +ve | +ve |
| | topsy | | | mths | | dystrophy | | |
| 5 | au- | A07-103 | 62 | 32 | M | Atypical | +ve | +ve |
| | topsy | | | mths | | FTLD-U | | |
| 6 | au- | A07-139 | 46 | 5 | M | FALS1 A4V | +ve | NT |
| | topsy | | | mths | | | | |
| 7 | au- | A07-141 | 83 | 48 | M | FLTD | −ve | NT |
| | topsy | | | mths | | (no UBIs) | | |
| 8 | au- | A08-016 | 65 | 17 | M | ALS with | +ve | NT |
| | topsy | | | mths | | early | | |
| | | | | | | FTLD-U | | |

ALS/FTLD-U (4): 3 +ve, 1 −ve
FTD (no UBIs) (1): 1 −ve
Possible ALS (1): 1 +ve
Non-ALS (2): 1 +ve: 1 −ve
Non-ALS (2): 1 +ve, 1 −ve
Pure FTD (no UBIs) (1)
ALS - Amyotrophic Lateral Sclerosis
FTLD - Frontotemporal Lobar Degeneration
FTLD-U - Frontotemporal Lobar Degeneration with ubiquitinated inclusions
UBIs - Ubiquitinated Inclusions
NT - Not Tested

### REFERENCES

Arai T, Hasegawa M, Akiyama H et al. TDP-43 is a component of ubiquitin-positive tau-negative inclusions in frontotemporal lobar degeneration and amyotrophic lateral sclerosis. Biochem Biophys Res Commun. 2006;351:602-611
Beaulieu J M, Robertson J, Julien J P. Interactions between peripherin and neurofilaments in cultured cells: disruption of peripherin assembly by the NF-M and NF-H subunits. Biochem Cell Biol. 1999;77:41-45
Buratti E, Dork T, Zuccato E et al. Nuclear factor TDP-43 and SR proteins promote in vitro and in vivo CFTR exon 9 skipping. Embo J. 2001;20:1774-1784
Carpenter, S. (1968). Proximal axonal enlargement in motor neuron disease. Neurology. 18:841-851
Carter, J., Gragerov, A., Konvicka, K., Elder, G., Weinstein, H. and Lazzarini, R. A. (1998). Neurofilament (NF) assembly; divergent characteristics of human and rodent NF-L subunits. J. Biol. Chem. 273:5101-5108
Clark F, Thanaraj T A. Categorization and characterization of transcript-confirmed constitutively and alternatively spliced introns and exons from human. Hum Mol Genet. 2002;11:451-464
Clement A M, Nguyen M D, Roberts E A et al. Wild-type nonneuronal cells extend survival of SOD1 mutant motor neurons in ALS mice. Science. 2003;302:113-117
Corbo M, Hays A P. Peripherin and neurofilament protein coexist in spinal spheroids of motor neuron disease. J Neuropathol Exp Neurol. 1992;51:531-537
De Jonghe, P., Mersivanova, I., Neilis, E., del Favero. J., Martin, J. J., van Broeckhoven, C., Evgrafov, O. and Timmerman, V. (2001). Further evidence that neurofilament light chain gene mutations can cause Charcot-Marie-Tooth disease type 2E. Ann. Neurol. 49:245-249
Ehling R, Lutterotti A, Wanschitz J, Khalil M, Gneiss C, Deisenhammer F, Reindl M, Berger T., Increased frequencies of serum antibodies to neurofilament light in patients with primary chronic progressive multiple sclerosis. Mult Scler. December 2004;10(6):601-6.
Fuchs, E. and Weber, K. (1994). Intermediate filaments: Structure, dynamics, function and disease. Annu. Rev. Biochem. 63:345-82
Hartmann, A. M., Rujescu, D., Giannakouros, T., Nikolakaki, E., Goedert, M., Mandelkow, E. M., Gao, Q. S., Andreadis, A. and Stamm, S. (2001). Regulation of alternative splicing of human tau exon 10 by phosphorylation of splicing factors. Mol. Cell. Neurosci. 18: 80-90
Hays A P. The Pathology of Amyotrophic Lateral Sclerosis. 1 vol. 1 ed. New York: Taylor & Francis, 2006:43-80
Hays A P, Naini A, He C Z et al. Sporadic amyotrophic lateral sclerosis and breast cancer: Hyaline conglomerate inclusions lead to identification of SOD1 mutation. J Neurol Sci. 2006;242:67-69
He C Z, Hays A P. Expression of peripherin in ubiquinated inclusions of amyotrophic lateral sclerosis. J Neurol Sci. 2004;217:47-54
Hernandez, F., Perez, M., Lucas, J. J., Mata, A. M., Bhat, R. and Avila, J. (2004). Glycogen synthase kinase-3 plays a crucial role in tau exon 10 splicing and intranuclear distribution of SC35. Implications for Alzheimer's disease. J. Biol. Chem. 279: 3801-3806

Ince P G, Lowe J, Shaw P J. Amyotrophic lateral sclerosis: current issues in classification, pathogenesis and molecular pathology. Neuropathol Appl Neurobiol. 1998;24:104-117
Ince P G, Tomkins J, Slade J Y et al. Amyotrophic lateral sclerosis associated with genetic abnormalities in the gene encoding Cu/Zn superoxide dismutase: molecular pathology of five new cases, and comparison with previous reports and 73 sporadic cases of ALS. J Neuropathol Exp Neurol. 1998;57:895-904
Kan Z, States D, Gish W. Selecting for functional alternative splices in ESTs. Genome Res. 2002;12:1837-1845
Karcher D, Federsppiel B S, Lowenthal F D, Frank F, Lowenthal A. Anti-neurofilament antibodies in blood of patients with neurological diseases. Acta Neuropathol (Berl). 1986;72(1):82-5.
Landon F, Lemonnier M, Benarous R et al. Multiple mRNAs encode peripherin, a neuronal intermediate filament protein. Embo J. 1989;8:1719-1726
Landon F, Wolff A, de Nechaud B. Mouse peripherin isoforms. Biol Cell. 2000;92:397-407
Lariviere R C, Beaulieu J M, Nguyen M D, Julien J P. Peripherin is not a contributing factor to motor neuron disease in a mouse model of amyotrophic lateral sclerosis caused by mutant superoxide dismutase. Neurobiol Dis. 2003;13:158-166
Lee, M. K., Marszalek, J. R. and Cleveland, D. W. (1994). A mutant neurofilament subunit causes massive, selective motor neuron death: implications for the pathogenesis of human motor neuron disease. Neuron. 13:975-988
Liem, R. K. (1993). Molecular biology of neuronal intermediate filaments. Curr. Opin. Cell Biol. 5:12-16
Mersiyanova, I. V., Perepelov, A. V., Polyakov, A. V., Sitnikov, V. F., Dadali, E. L., Oparin, R. B., Petrin, A. N. and Evgrafov, O. V. (2000). A new variant of Charcot-Marie-Tooth disease type 2 is probably the result a mutation in the neurofilament-light gene. Am. J. Hum. Genet. 67:37-46
Migheli A, Pezzulo T, Attanasio A, Schiffer D. Peripherin immunoreactive structures in amyotrophic lateral sclerosis. Lab Invest. 1993;68:185-191
Neumann M, Sampathu D M, Kwong L K et al. Ubiqitinated TDP-43 in frontotemporal lobar degeneration and amyotrophic lateral sclerosis. Science. 2006
Perez-Olle, R., Leung, C. L. and Liem, R. K. (2002). Effects of Charcot-Marie-Tooth-linked mutations of the neurofilament light subunit on intermediate filament formation. J. Cell Sci. 115:4937-4946
Puertas M C, Carrillo J, Pastor X, Ampudia R M, Planas R, Alba A, Bruno R, Pujol-Borrell R, Estanyol J M, Vives-Pi M, Verdaguer J. Peripherin is a relevant neuroendocrine autoantigen recognized by islet-infiltrating B lymphocytes. J Immunol. May 15, 2007;178(10):6533-9.
Rosen D R, Siddique T, Patterson D et al. Mutations in Cu/Zn superoxide dismutase gene are associated with familial amyotrophic lateral sclerosis. Nature. 1993;362:59-62
Robertson J, Doroudchi M M, Nguyen M D et al. A neurotoxic peripherin splice variant in a mouse model of ALS. J Cell Biol. 2003;160:939-949
Sarria A J, Lieber J G, Nordeen S K, Evans R M. The presence or absence of a vimentin-type intermediate filament network affects the shape of the nucleus in human SW-13 cells. J Cell Sci. 1994;107 (Pt 6):1593-1607
Schwartz, M. L., Bruce, J., Shneidman, P. S. and Schlaepfer, W. W. (1995). Deletion of 3′-untranslated region alters the level of mRNA expression of a neurofilament light subunit transgene. J. Biol. Chem. 270:26364-26369
Stoilov, P., Meshorer, E., Gencheva, M., Glick, D., Soreq, H. and Stamm, S. (2002). Defects in pre-mRNA processing as causes of and predisposition to diseases. DNA Cell Biol. 21: 803-818
Wong J, Oblinger M M. Differential regulation of peripherin and neurofilament gene expression in regenerating rat DRG neurons. J Neurosci Res. 1990;27:332-341
Xiao S, McLean J, Robertson J. Neuronal intermediate filaments and ALS: A new look at an old question. Biochim Biophys Acta. 2006

1. An isolated nucleic acid molecule comprising
(a) a nucleic acid sequence as shown in SEQ ID NO:2, 27, 7, or 9, wherein T can also be U;
(b) a nucleic acid sequence that is complementary to a nucleic acid sequence of (a);
(c) a nucleic acid sequence that has substantial sequence homology to a nucleic acid sequence of (a) or (b);
(d) a nucleic acid sequence that is an analog of a nucleic acid sequence of (a), (b) or (c);
(e) a nucleic acid sequence that hybridizes to a nucleic acid sequence of (a), (b), (c) or (d) under stringent hybridization conditions; and
(f) a nucleic acid sequence differing from any of the nucleic acid sequences of (a) to (e) in codon sequences due to the degeneracy of the genetic code.

2. An isolated nucleic acid molecule encoding an amino acid sequence as shown in SEQ ID NOs:5, 28, 7 or 9.

3. A recombinant expression vector comprising the isolated nucleic acid molecule of claim 1.

4. An siRNA molecule comprising the RNA sequence as shown in SEQ ID NOs: 19-22 or SEQ ID NOs: 23 and 24 or SEQ ID NOs:25 and 26.

5. An isolated polypeptide comprising the amino acid sequence as shown in SEQ ID NOs:5, 28, 7 or 9 or a fragment thereof.

6. The isolated polypeptide of claim 5, wherein the fragment comprises VSGPGIRGGF (SEQ ID NO:4).

7. The isolated polypeptide of claim 5, wherein the fragment comprises VQEPGGPARRDAGVVSRVPAD (SEQ ID NO:29).

8. The isolated polypeptide of claim 5, wherein the fragment comprises NDLKSIRDLR (SEQ ID NO:8).

9. The isolated polypeptide of claim 5, wherein the fragment comprises ARKGAKNTDA (SEQ ID NO:10).

10. A binding protein that binds to the isolated polypeptide of claim 5.

11. The binding protein of claim 10, wherein the binding protein is an antibody or fragment thereof.

12. The binding protein of claim 11, wherein the antibody is a monoclonal antibody.

13. A method of detecting ALS in a subject suspected of having ALS, comprising detecting the presence or absence of a nucleotide sequence or a change in the amount of the nucleic acid sequence encoding the polypeptide having the amino acid sequence as shown in SEQ ID NO:5, 28, 7 or 9; wherein the presence or change in the amount of the nucleotide sequence is indicative of ALS.

14. The method of claim 13, wherein detecting the presence or absence or change in the amount of the nucleotide sequence comprises contacting the sample under hybridization conditions with one or more nucleotide probes labeled with a detectable marker.

15. A method of detecting, monitoring or diagnosing ALS in a subject comprising the steps of:
(a) contacting a sample of said subject with the binding protein of claim 10;
(b) measuring the amount of binding protein-protein complex in the sample; and
(c) comparing the amount of binding protein-protein complex in the sample to a control;
wherein a difference in the amount of binding protein-protein complex in the sample as compared to control is indicative of ALS or the stage of ALS.

16. The method of claim 15, wherein the difference in the amount of binding protein-protein complex is an increase.

17. The method of claim 15, wherein the difference in the amount of binding protein-protein complex is a decrease.

18. The method of claim 15, wherein the sample comprises cerebrospinal fluid, plasma, blood serum, whole blood, spinal cord tissue, brain cells, motor neurons, urine or peripheral blood cells.

19. The method of claim 15, wherein the subject is an animal.

20. The method of claim 19, wherein the animal is human.

21. The method of claim 15, further comprising assessing the subject using traditional techniques for ALS.

22. A kit comprising the binding protein of claim 10 and instructions for use.

23. The kit of claim 22, wherein the binding protein is labeled.
